(19) United States
(12) Patent Application Publication
(10) Pub. No.: US 2025/0041558 A1
(43) Pub. Date: Feb. 6, 2025

(54) METHOD AND SYSTEM THAT MONITOR MENTAL STATES OF USERS INTERACTING WITH ELECTRONIC DEVICES AND THAT SAFELY PROVIDE STIMULI TO THE USER

(71) Applicant: Craig Alan Friedman, New York, NY (US)

(72) Inventor: Craig Alan Friedman, New York, NY (US)

(21) Appl. No.: 18/659,849

(22) Filed: May 9, 2024

Related U.S. Application Data
(60) Provisional application No. 63/465,217, filed on May 9, 2023.

(51) Int. Cl. A61M 21/02; A61M 21/00; G16H 20/70; G16H 50/20

(52) U.S. Cl. CPC A61M 21/02; G16H 20/70; G16H 50/20; A61M 2021/0027; A61M 2021/005; A61M 2230/04; A61M 2230/20; A61M 2230/30; A61M 2230/50

(57) ABSTRACT

The current document is directed to devices and systems that provide instructional, therapeutic, and other goal-directed information to users of electronic devices while, at the same time, monitoring the users in order to track their mental states and to provide stimuli to the users to further instructional, therapeutical, and other goals associated with information provision. In a disclosed implementation, a remote, distributed mental-state-monitoring and stimulus-directing application continuously receives various types of monitoring signals from users' computers and other electronic systems while the users are receiving the goal-directed information and, in many cases, interacting with a remote information-provision system. The monitoring signals are used by the mental-state-monitoring and stimulus-directing application to continuously evaluate users' mental states, detect various types of problems, and to direct users' computers and other electronic devices to provide stimuli to the users in order to ameliorate the detected problems.

### CROSS-REFERENCE TO RELATED APPLICATION

This application claims the benefit of Provisional Application No. 63/465,217, filed May 9, 2023.

### TECHNICAL FIELD

The current document is directed to devices and systems that provide instruction, therapy, and other goal-directed information to users of electronic devices, including personal computers and smartphones, and, in particular, to methods and systems that monitor the users in order to track their mental states and to provide stimuli to the users to further instructional, therapeutical, and other goals associated with information provision.

### BACKGROUND

During the past seven decades, electronic computing has evolved from primitive, vacuum-tube-based computer systems, initially developed during the 1940s, to modern electronic computing systems that include powerful and capable personal computers, laptops, and smartphones as well as large, geographically distributed computer systems comprising large numbers of networked multi-processor servers, work stations, and other individual computing systems to provide enormous computational bandwidths and data-storage capacities. As the computational bandwidths and networking bandwidths of computer systems and communications subsystems have increased, both individual computer systems and distributed computer systems have evolved to provide for parallel execution of a wide variety of complex applications that can access large volumes of stored data and interact with large numbers of different computer systems and users of the computer systems. These capabilities provide the basis for sophisticated instructional systems, in which remote students can participate in virtual classrooms, passively receive large volumes of instructional programs, and interact with remote instructional systems. These capabilities additionally provide the basis for sophisticated diagnostic and therapeutic systems and a wide variety of additional types of information sources.

In classrooms, medical facilities, and counseling facilities, when in-person construction, diagnosis, and therapy is conducted, human professionals can continuously monitor students and patients, in real time, in order to continuously ascertain the effectiveness of information transfer to students and patients, detect problems, and alter the information provided to students and patients as well as various approaches to information provision in order to attempt to optimize instruction, diagnosis, therapy, and other goals underlying information provision and interaction. However, when information is provided through electronic systems to remote students and patients, the ability for continuous, real-time monitoring of the students and patients by human professionals is greatly constrained and often nearly impossible. Thus, current instructional, diagnostic, and therapeutic systems that provide instruction, diagnosis, and therapy to remote students and patients are relatively well-developed and effective with respect to information provision from remote systems to students and patients, but lack effective collection of feedback information from the students and patients and an ability to use the feedback information to modify and tailor subsequent information provision in order to address inefficiencies and problems experienced by students and patients. Designers, developers, and vendors of information-provision systems have thus recognized a need to improve the collection and use of feedback from remote users in order to more effectively provide instructional, diagnostic, and therapeutic information to remote users.

### SUMMARY

The current document is directed to devices and systems that provide instructional, therapeutic, and other goal-directed information to users of electronic devices while, at the same time, monitoring the users in order to track their mental states and to provide stimuli to the users to further instructional, therapeutical, and other goals associated with information provision. In a disclosed implementation, a remote, distributed mental-state-monitoring and stimulus-directing application continuously receives various types of monitoring signals from users' computers and other electronic systems while the users are receiving the goal-directed information and, in many cases, interacting with a remote information-provision system. The monitoring signals are used by the mental-state-monitoring and stimulus-directing application to continuously evaluate users' mental states, detect various types of problems, and to direct users' computers and other electronic devices to provide stimuli to the users in order to ameliorate the detected problems.

### BRIEF DESCRIPTION OF THE DRAWINGS

FIG. 1 provides a general architectural diagram for various types of computers.

FIG. 2 illustrates an Internet-connected distributed computer system.

FIG. 3 illustrates cloud computing.

FIG. 4 illustrates generalized hardware and software components of a general-purpose computer system, such as a general-purpose computer system having an architecture similar to that shown in FIG. 1.

FIGS. 5A-B illustrate two types of virtual machine and virtual-machine execution environments.

FIGS. 6A-C illustrate traditional classroom instruction.

FIGS. 7A-B illustrates diagnostic and therapeutic interactions between a professional and a patient.

FIGS. 8A-C illustrates information provision in remote-learning and remote-diagnosis contexts.

FIG. 9A-B illustrates one of many different types of physiology-sensing computer peripherals that are currently available.

FIG. 10 shows components of, and associated with, an example mental-state-monitoring and stimulus-directing system that represents one implementation of the currently disclosed methods and systems.

FIGS. 11A-B illustrate data pathways within the user's computer system and between the sensor-containing headband and wristband and the user's computer system in the MM/SD system introduced with reference to FIG. 10.

FIGS. 12A-E illustrate operation of the MM/SD system introduced with reference to FIGS. 10-11B.

FIGS. 13-20 illustrate one implementation of the MM/SD system.

FIG. 14 illustrates one implementation of an SP/SD component.

FIG. 15 illustrates the signal-demultiplexer component (1404 in FIG. 14) of an SP/SD component of the DMS application.

FIG. 16 illustrates an attribute-detector component of an SP/SD component of the DMS application.

FIG. 17 illustrates the mental-state-synthesizer and stimulus-director component (1420 in FIG. 14) of an SP/SD component of the DMS application.

FIG. 18 provides a control-flow diagram that illustrates operation of the controller (1304 in FIG. 13) of the DMS application.

FIG. 19 provides a control-flow diagram that illustrates operation of the CMS application (1212 in FIG. 12B).

FIG. 20 illustrates fundamental components of a feed-forward neural network.

FIGS. 21A-J illustrate operation of a very small, example neural network.

FIGS. 22A-C show details of the computation of weight adjustments made by neural-network nodes during backpropagation of error vectors into neural networks.

FIGS. 23A-B illustrate neural-network training.

FIGS. 24A-F illustrate a matrix-operation-based batch method for neural-network training.

FIGS. 25A-C illustrate various aspects of recurrent neural networks.

FIGS. 26A-C illustrate a convolutional neural network.

### DETAILED DESCRIPTION

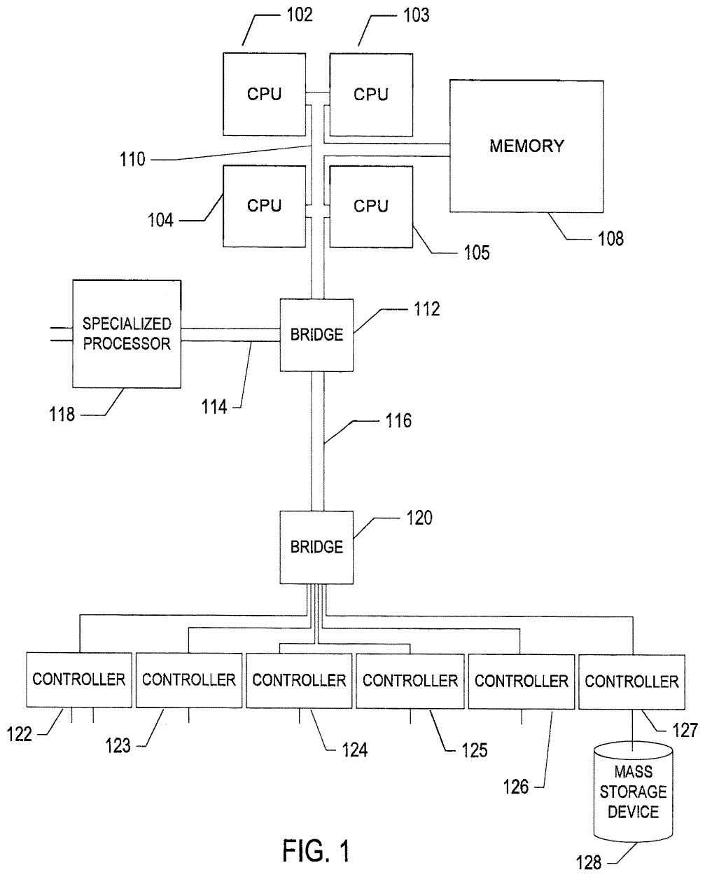

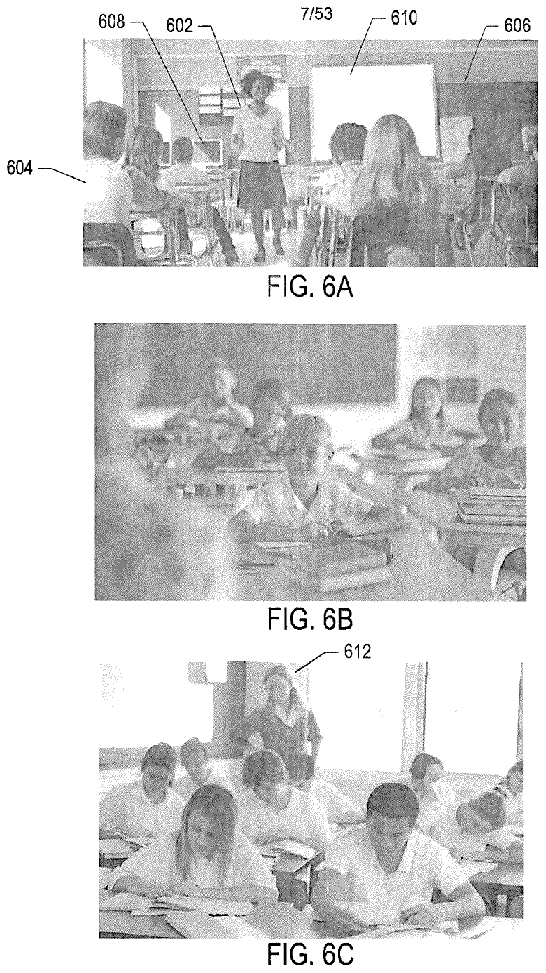

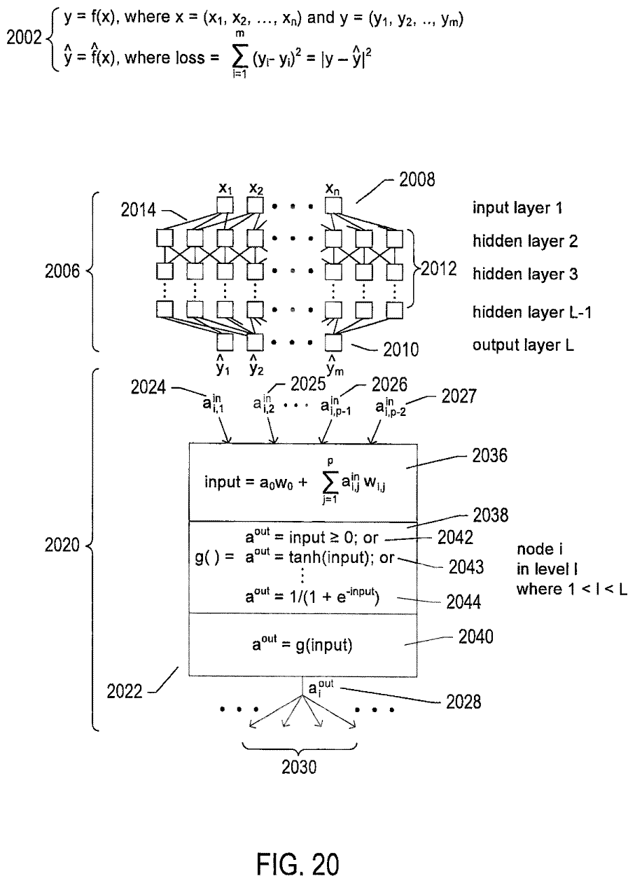

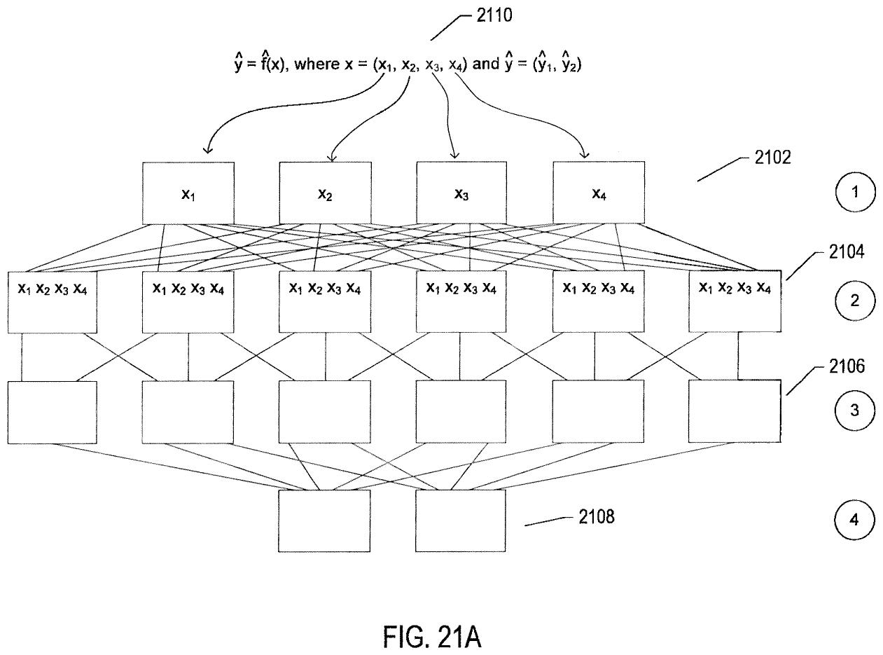

The current document is directed to devices and systems that provide instructional, therapeutic, and other goal-directed information to users of electronic devices while, at the same time, monitoring the users in order to track their mental states and to provide stimuli to the users to further instructional, therapeutical, and other goals associated with information provision. In a first subsection, below, an overview of computer hardware, complex computational systems, and virtualization is provided with reference to FIGS. 1-5B. In a second subsection, an overview of the currently disclosed methods and systems is provided with reference to FIGS. 6A-19. In a third subsection, neural networks are discussed with reference to FIGS. 20-26B. In a fourth subsection, details regarding the neural networks used in a disclosed implementation of the currently disclosed methods and systems are provided.

### Computer Hardware, Complex Computational Systems, and Virtualization

The term “abstraction” is not, in any way, intended to mean or suggest an abstract idea or concept. Computational abstractions are tangible, physical interfaces that are implemented, ultimately, using physical computer hardware, data-storage devices, and communications systems. Instead, the term “abstraction” refers, in the current discussion, to a logical level of functionality encapsulated within one or more concrete, tangible, physically-implemented computer systems with defined interfaces through which electronically-encoded data is exchanged, process execution launched, and electronic services are provided. Interfaces may include graphical and textual data displayed on physical display devices as well as computer programs and routines that control physical computer processors to carry out various tasks and operations and that are invoked through electronically implemented application programming interfaces (“APIs”) and other electronically implemented interfaces. There is a tendency among those unfamiliar with modern technology and science to misinterpret the terms “abstract” and “abstraction,” when used to describe certain aspects of modern computing. For example, one frequently encounters assertions that, because a computational system is described in terms of abstractions, functional layers, and interfaces, the computational system is somehow different from a physical machine or device. Such allegations are unfounded. One only needs to disconnect a computer system or group of computer systems from their respective power supplies to appreciate the physical, machine nature of complex computer technologies. One also frequently encounters statements that characterize a computational technology as being “only software,” and thus not a machine or device. Software is essentially a sequence of encoded symbols, such as a printout of a computer program or digitally encoded computer instructions sequentially stored in a file on an optical disk or within an electromechanical mass-storage device. Software alone can do nothing. It is only when encoded computer instructions are loaded into an electronic memory within a computer system and executed on a physical processor that so-called “software implemented” functionality is provided. The digitally encoded computer instructions are an essential and physical control component of processor-controlled machines and devices, no less essential and physical than a cam-shaft control system in an internal-combustion engine. Multi-cloud aggregations, cloud-computing services, virtual-machine containers and virtual machines, communications interfaces, and many of the other topics discussed below are tangible, physical components of physical, electro-optical-mechanical computer systems.

FIG. 1 provides a general architectural diagram for various types of computers. The computer system contains one or multiple central processing units (“CPUs”) 102-105, one or more electronic memories 108 interconnected with the CPUs by a CPU/memory-subsystem bus 110 or multiple buses, a first bridge 112 that interconnects the CPU/memory-subsystem bus 110 with additional buses 114 and 116, or other types of high-speed interconnection media, including multiple, high-speed serial interconnects. These buses or serial interconnections, in turn, connect the CPUs and memory with specialized processors, such as a graphics processor 118, and with one or more additional bridges 120, which are interconnected with high-speed serial links or with multiple controllers 122-127, such as controller 127, that provide access to various different types of mass-storage devices 128, electronic displays, input devices, and other such components, subcomponents, and computational resources. It should be noted that computer-readable data-storage devices include optical and electromagnetic disks, electronic memories, and other physical data-storage devices. Those familiar with modern science and technology appreciate that electromagnetic radiation and propagating signals do not store data for subsequent retrieval and can transiently “store” only a byte or less of information per mile, far less information than needed to encode even the simplest of routines. The various types of computers, including personal computers, laptops, smartphones, workstations, tablets, and other such devices used by individuals may be referred to as “processor-controlled devices” or “processor-controlled appliances.”

Of course, there are many different types of computer-system architectures that differ from one another in the number of different memories, including different types of hierarchical cache memories, the number of processors and the connectivity of the processors with other system components, the number of internal communications buses and serial links, and in many other ways. However, computer systems generally execute stored programs by fetching instructions from memory and executing the instructions in one or more processors. Computer systems include general-purpose computer systems, such as personal computers (“PCs”), various types of servers and workstations, and higher-end mainframe computers, but may also include a plethora of various types of special-purpose computing devices, including data-storage systems, communications routers, network nodes, tablet computers, and mobile telephones.

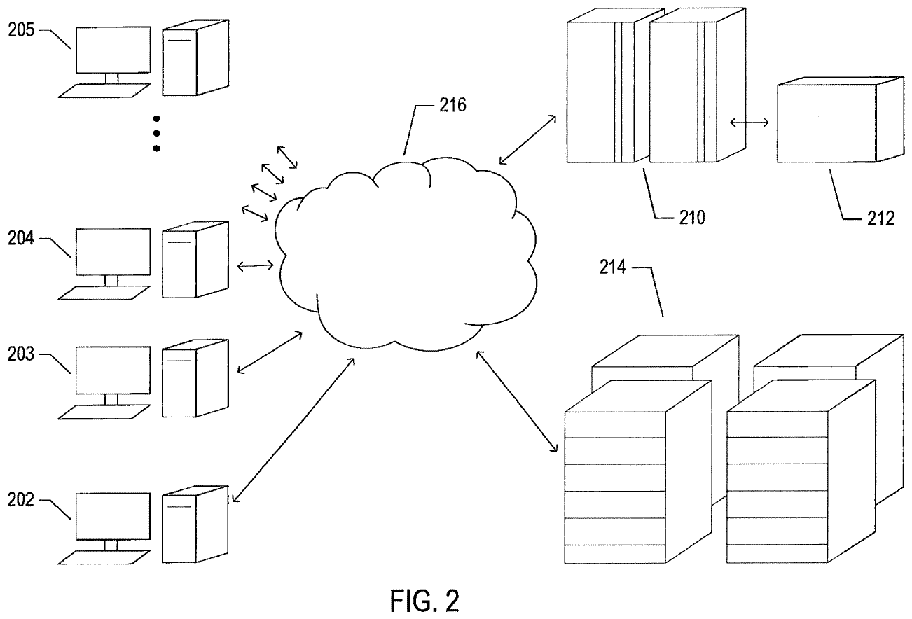

FIG. 2 illustrates an Internet-connected distributed computer system. As communications and networking technologies have evolved in capability and accessibility, and as the computational bandwidths, data-storage capacities, and other capabilities and capacities of various types of computer systems have steadily and rapidly increased, much of modern computing now generally involves large distributed systems and computers interconnected by local networks, wide-area networks, wireless communications, and the Internet. FIG. 2 shows a typical distributed system in which a large number of PCs 202-205, a high-end distributed mainframe system 210 with a large data-storage system 212, and a large computer center 214 with large numbers of rack-mounted servers or blade servers all interconnected through various communications and networking systems that together comprise the Internet 216. Such distributed computer systems provide diverse arrays of functionalities. For example, a PC user sitting in a home office may access hundreds of millions of different web sites provided by hundreds of thousands of different web servers throughout the world and may access high-computational-bandwidth computing services from remote computer facilities for running complex computational tasks.

Until recently, computational services were generally provided by computer systems and data centers purchased, configured, managed, and maintained by service-provider organizations. For example, an e-commerce retailer generally purchased, configured, managed, and maintained a data center including numerous web servers, back-end computer systems, and data-storage systems for serving web pages to remote customers, receiving orders through the web-page interface, processing the orders, tracking completed orders, and other myriad different tasks associated with an e-commerce enterprise.

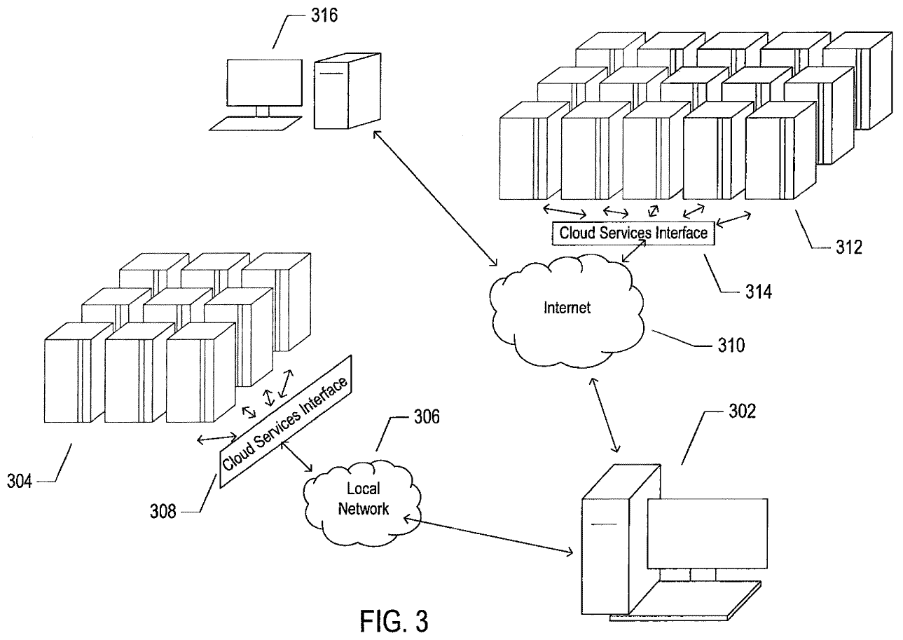

FIG. 3 illustrates cloud computing. In the recently developed cloud-computing paradigm, computing cycles and data-storage facilities are provided to organizations and individuals by cloud-computing providers. In addition, larger organizations may elect to establish private cloud-computing facilities in addition to, or instead of, subscribing to computing services provided by public cloud-computing service providers. In FIG. 3, a system administrator for an organization, using a PC 302, accesses the organization's private cloud 304 through a local network 306 and private-cloud interface 308 and also accesses, through the Internet 310, a public cloud 312 through a public-cloud services interface 314. The administrator can, in either the case of the private cloud 304 or public cloud 312, configure virtual computer systems and even entire virtual data centers and launch execution of application programs on the virtual computer systems and virtual data centers in order to carry out any of many different types of computational tasks. As one example, a small organization may configure and run a virtual data center within a public cloud that executes web servers to provide an e-commerce interface through the public cloud to remote customers of the organization, such as a user viewing the organization's e-commerce web pages on a remote user system 316.

Cloud-computing facilities are intended to provide computational bandwidth and data-storage services much as utility companies provide electrical power and water to consumers. Cloud computing provides enormous advantages to small organizations without the resources to purchase, manage, and maintain in-house data centers. Such organizations can dynamically add and delete virtual computer systems from their virtual data centers within public clouds in order to track computational-bandwidth and data-storage needs, rather than purchasing sufficient computer systems within a physical data center to handle peak computational-bandwidth and data-storage demands. Moreover, small organizations can completely avoid the overhead of maintaining and managing physical computer systems, including hiring and periodically retraining information-technology specialists and continuously paying for operating-system and database-management-system upgrades. Furthermore, cloud-computing interfaces allow for easy and straightforward configuration of virtual computing facilities, flexibility in the types of applications and operating systems that can be configured, and other functionalities that are useful even for owners and administrators of private cloud-computing facilities used by a single organization.

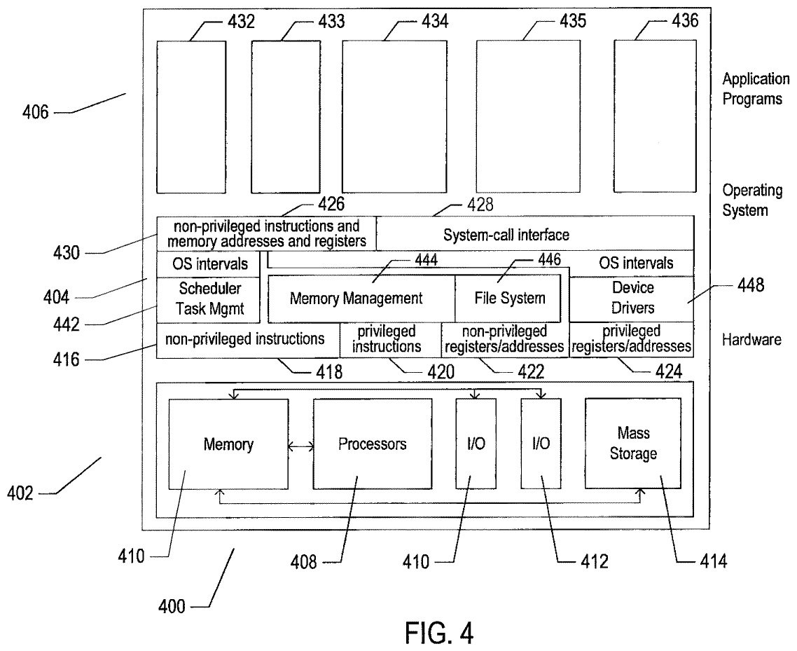

FIG. 4 illustrates generalized hardware and software components of a general-purpose computer system, such as a general-purpose computer system having an architecture similar to that shown in FIG. 1. The computer system 400 is often considered to include three fundamental layers: (1) a hardware layer or level 402; (2) an operating-system layer or level 404; and (3) an application-program layer or level 406. The hardware layer 402 includes one or more processors 408, system memory 410, various different types of input-output (“I/O”) devices 410 and 412, and mass-storage devices 414. Of course, the hardware level also includes many other components, including power supplies, internal communications links and buses, specialized integrated circuits, many different types of processor-controlled or microprocessor-controlled peripheral devices and controllers, and many other components. The operating system 404 interfaces to the hardware level 402 trough a low-level operating system and hardware interface 416 generally comprising a set of non-privileged computer instructions 418, a set of privileged computer instructions 420, a set of non-privileged registers and memory addresses 422, and a set of privileged registers and memory addresses 424. In general, the operating system exposes non-privileged instructions, non-privileged registers, and non-privileged memory addresses 426 and a system-call interface 428 as an operating-system interface 430 to application programs 432-436 that execute within an execution environment provided to the application programs by the operating system. The operating system, alone, accesses the privileged instructions, privileged registers, and privileged memory addresses. By reserving access to privileged instructions, privileged registers, and privileged memory addresses, the operating system can ensure that application programs and other higher-level computational entities cannot interfere with one another's execution and cannot change the overall state of the computer system in ways that could deleteriously impact system operation. The operating system includes many internal components and modules, including a scheduler 442, memory management 444, a file system 446, device drivers 448, and many other components and modules. To a certain degree, modern operating systems provide numerous levels of abstraction above the hardware level, including virtual memory, which provides to each application program and other computational entities a separate, large, linear memory-address space that is mapped by the operating system to various electronic memories and mass-storage devices. The scheduler orchestrates interleaved execution of various different application programs and higher-level computational entities, providing to each application program a virtual, stand-alone system devoted entirely to the application program. From the application program's standpoint, the application program executes continuously without concern for the need to share processor resources and other system resources with other application programs and higher-level computational entities. The device drivers abstract details of hardware-component operation, allowing application programs to employ the system-call interface for transmitting and receiving data to and from communications networks, mass-storage devices, and other I/O devices and subsystems. The file system 436 facilitates abstraction of mass-storage-device and memory resources as a high-level, easy-to-access, file-system interface. Thus, the development and evolution of the operating system has resulted in the generation of a type of multi-faceted virtual execution environment for application programs and other higher-level computational entities.

While the execution environments provided by operating systems have proved to be an enormously successful level of abstraction within computer systems, the operating-system-provided level of abstraction is nonetheless associated with difficulties and challenges for developers and users of application programs and other higher-level computational entities. One difficulty arises from the fact that there are many different operating systems that run within various different types of computer hardware. In many cases, popular application programs and computational systems are developed to run on only a subset of the available operating systems and can therefore be executed within only a subset of the various different types of computer systems on which the operating systems are designed to run. Often, even when an application program or other computational system is ported to additional operating systems, the application program or other computational system can nonetheless run more efficiently on the operating systems for which the application program or other computational system was originally targeted. Another difficulty arises from the increasingly distributed nature of computer systems. Although distributed operating systems are the subject of considerable research and development efforts, many of the popular operating systems are designed primarily for execution on a single computer system. In many cases, it is difficult to move application programs, in real time, between the different computer systems of a distributed computer system for high-availability, fault-tolerance, and load-balancing purposes. The problems are even greater in heterogeneous distributed computer systems which include different types of hardware and devices running different types of operating systems. Operating systems continue to evolve, as a result of which certain older application programs and other computational entities may be incompatible with more recent versions of operating systems for which they are targeted, creating compatibility issues that are particularly difficult to manage in large distributed systems.

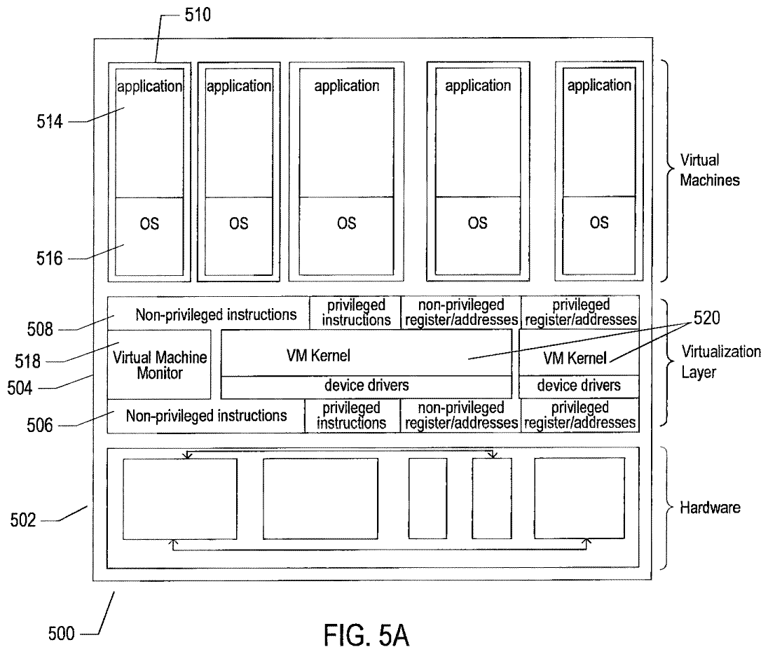

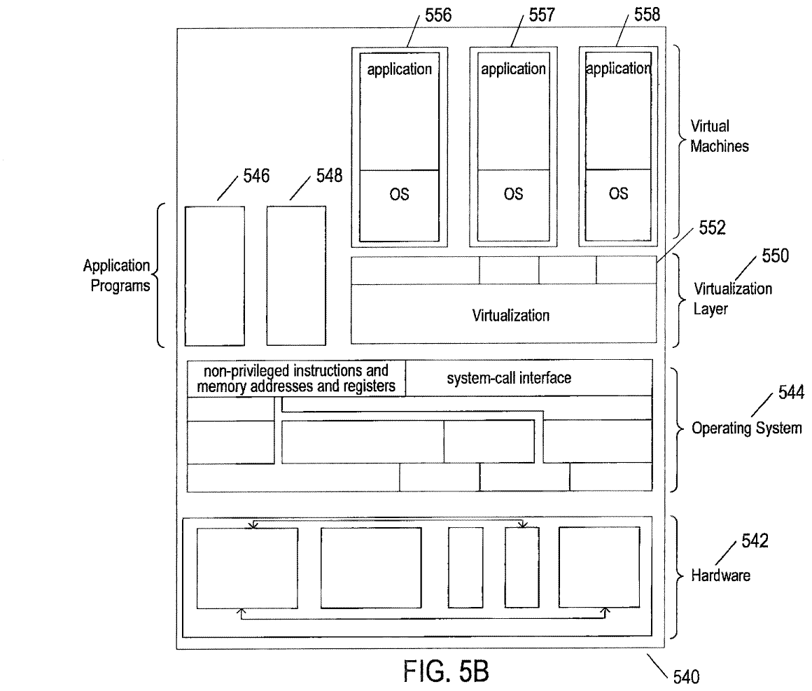

For all of these reasons, a higher level of abstraction, referred to as the “virtual machine,” has been developed and evolved to further abstract computer hardware in order to address many difficulties and challenges associated with traditional computing systems, including the compatibility issues discussed above. FIGS. 5A-B illustrate several types of virtual machine and virtual-machine execution environments. FIGS. 5A-B use the same illustration conventions as used in FIG. 4. FIG. 5A shows a first type of virtualization. The computer system 500 in FIG. 5A includes the same hardware layer 502 as the hardware layer 402 shown in FIG. 4. However, rather than providing an operating system layer directly above the hardware layer, as in FIG. 4, the virtualized computing environment illustrated in FIG. 5A features a virtualization layer 504 that interfaces through a virtualization-layer/hardware-layer interface 506, equivalent to interface 416 in FIG. 4, to the hardware. The virtualization layer provides a hardware-like interface 508 to a number of virtual machines, such as virtual machine 510, executing above the virtualization layer in a virtual-machine layer 512. Each virtual machine includes one or more application programs or other higher-level computational entities packaged together with an operating system, referred to as a “guest operating system,” such as application 514 and guest operating system 516 packaged together within virtual machine 510. Each virtual machine is thus equivalent to the operating-system layer 404 and application-program layer 406 in the general-purpose computer system shown in FIG. 4. Each guest operating system within a virtual machine interfaces to the virtualization-layer interface 508 rather than to the actual hardware interface 506. The virtualization layer partitions hardware resources into abstract virtual-hardware layers to which each guest operating system within a virtual machine interfaces. The guest operating systems within the virtual machines, in general, are unaware of the virtualization layer and operate as if they were directly accessing a true hardware interface. The virtualization layer ensures that each of the virtual machines currently executing within the virtual environment receive a fair allocation of underlying hardware resources and that all virtual machines receive sufficient resources to progress in execution. The virtualization-layer interface 508 may differ for different guest operating systems. For example, the virtualization layer is generally able to provide virtual hardware interfaces for a variety of different types of computer hardware. This allows, as one example, a virtual machine that includes a guest operating system designed for a particular computer architecture to run on hardware of a different architecture. The number of virtual machines need not be equal to the number of physical processors or even a multiple of the number of processors.

The virtualization layer includes a virtual-machine-monitor module 518 (“VMM”) that virtualizes physical processors in the hardware layer to create virtual processors on which each of the virtual machines executes. For execution efficiency, the virtualization layer attempts to allow virtual machines to directly execute non-privileged instructions and to directly access non-privileged registers and memory. However, when the guest operating system within a virtual machine accesses virtual privileged instructions, virtual privileged registers, and virtual privileged memory through the virtualization-layer interface 508, the accesses result in execution of virtualization-layer code to simulate or emulate the privileged resources. The virtualization layer additionally includes a kernel module 520 that manages memory, communications, and data-storage machine resources on behalf of executing virtual machines (“VM kernel”). The VM kernel, for example, maintains shadow page tables on each virtual machine so that hardware-level virtual-memory facilities can be used to process memory accesses. The VM kernel additionally includes routines that implement virtual communications and data-storage devices as well as device drivers that directly control the operation of underlying hardware communications and data-storage devices. Similarly, the VM kernel virtualizes various other types of I/O devices, including keyboards, optical-disk drives, and other such devices. The virtualization layer essentially schedules execution of virtual machines much like an operating system schedules execution of application programs, so that the virtual machines each execute within a complete and fully functional virtual hardware layer.

FIG. 5B illustrates a second type of virtualization. In FIG. 5B, the computer system 540 includes the same hardware layer 542 and software layer 544 as the hardware layer 402 shown in FIG. 4. Several application programs 546 and 548 are shown running in the execution environment provided by the operating system. In addition, a virtualization layer 550 is also provided, in computer 540, but, unlike the virtualization layer 504 discussed with reference to FIG. 5A, virtualization layer 550 is layered above the operating system 544, referred to as the “host OS,” and uses the operating system interface to access operating-system-provided functionality as well as the hardware. The virtualization layer 550 comprises primarily a VMM and a hardware-like interface 552, similar to hardware-like interface 508 in FIG. 5A. The virtualization-layer/hardware-layer interface 552, equivalent to interface 416 in FIG. 4, provides an execution environment for a number of virtual machines 556-558, each including one or more application programs or other higher-level computational entities packaged together with a guest operating system.

### Currently Disclosed Methods and Subsystems

FIGS. 6A-C illustrate traditional classroom instruction. In FIG. 6A, a teacher 602 is providing classroom instruction to multiple different students, including student 604. The teacher is speaking to the students but can provide information to the students using a variety of different devices, including a chalkboard 606, interactive computer systems 608, and an audio/visual display 610. As shown in FIG. 6B, the teacher sees, and constantly visually monitors, the students in her classroom. As shown in FIG. 6C, a teacher 612 can also monitor students as they work on in-classroom assignments and take exams. The teacher is able to acquire and process many different types of information with regard to her students, including facial expressions, posture, their apparent engagement with the information currently being provided to them, signs of attentiveness, distraction, frustration with, and lack of understanding, of the information provided to them, embarrassment and uneasiness, and many other types of information. Using the acquired information, the teacher is able to, in real time, alter the information provided to the students, alter the methods used by the teacher to provide the information, provide additional details with regard to subject matter that appears not to have been effectively communicated to the students, and provide various types of stimuli to the students to facilitate communication, including changing the rate of information provision, changing the voice tones used to communicate information, moving about the classroom to draw the students' attention, adding humorous anecdotes or additional examples to facilitate understanding, and other types of stimuli and feedback. Of course, the teacher is also aware of negative consequences of certain types of stimulus, such as scolding students or making statements that might trigger damaging emotional reactions, and must carefully consider the potential for negative stimulus at all times.

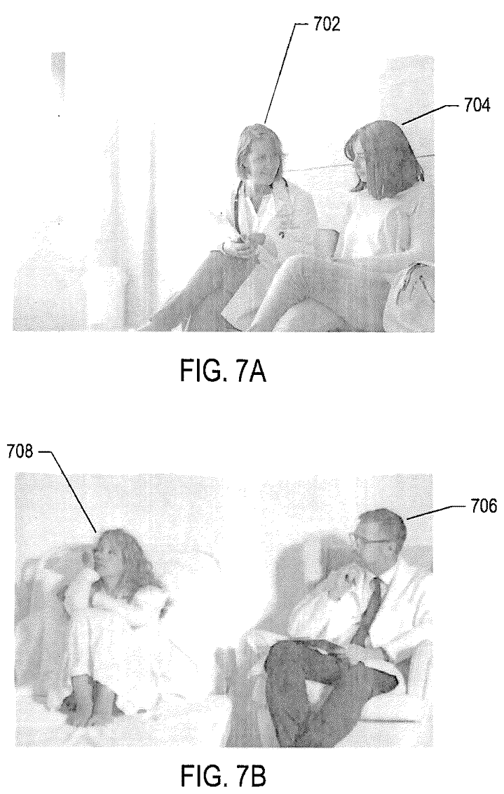

FIGS. 7A-B illustrates diagnostic and therapeutic interactions between a professional and a patient. In FIG. 7A, a medical doctor 702 discusses symptoms with a patient 704. In FIG. 7B, a therapist is attempting to inquire about a patient's 708 recollection of events related to a problematic relationship. In both cases, as with the teachers discussed with reference to FIGS. 6A-C, the professional is able to, in real time, observe many different types of information with respect to the patient, including facial expressions, verbal expressions, posture, physiological changes, attentiveness, frustration, confusion, and a variety of additional types of emotions, emotional responses, and physiological conditions. Using this information, the professional can, in real time, alter the professional's approaches in order to achieve one or more goals. When the professional, for example, detects that the patient is embarrassed by a particular line of questioning, the professional may temporarily change the subject and then try a different line of questioning in order to obtain the desired information. The professional may alter his or her verbal expressions, voice tones, posture, facial expressions, and other features to stimulate the patient and to elicit particular emotional responses and the patient. As with the teacher discussed in FIGS. 6A-C, the professional must constantly consider possible negative consequences of his or her actions and statements as well as devise strategies for positive stimulation.

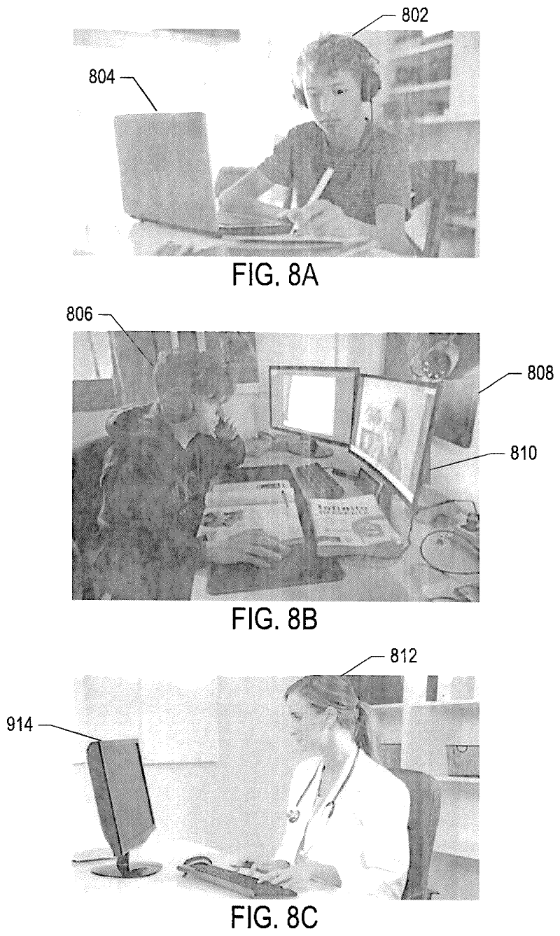

FIGS. 8A-C illustrates information provision in remote-learning and remote-diagnosis contexts. In FIG. 8A, a student 802 is receiving instruction from a remote information source via a laptop 804. In this case, the student receives visual and audio information and is taking notes, much as the student would listen to a lecture and take notes in a classroom. However, the information-exchange, in this case, is almost exclusively one-sided. The student lacks any means for providing feedback to the remote information source. Much like an undergraduate student in a large lecture hall listening to a lecture attended by hundreds of students, the student has very little ability to provide meaningful feedback to the lecturer. Of course, were all the students to groan when the lecturer announces a midterm the following week, the lecturer might become aware of a collective dissatisfaction with the midterm schedule, but an individual student's failure to understand the information being communicated by the lecturer would generally not be apparent to the lecturer.

In contrast to the one-sided information transfer discussed above with reference to FIG. 8A, FIG. 8B shows an interactive learning session in which a student 806 interacts with a human instructor 8:08 PM the student's computer system 810. In this case, the student is able to provide feedback to the instructor by speaking to the instructor through a computer-system microphone and by the instructor viewing the student through the camera incorporated in the student's computer system. However, in order for the instructor to receive the feedback, the instructor needs to interact exclusively with the single student or, in certain cases, with perhaps two or three students that the instructor views in two or three different windows displayed to the instructor on the instructor's computer system. This is not a terribly efficient type of instruction and more like individual tutoring. Furthermore, due to bandwidth and information-presentation constraints, the instructor may be unable to observe subtle facial expressions, physiological changes, voice tones, and other information that would be observed by the instructor in an in-person instruction session.

The medical professional 812 in FIG. 8C experiences similar problems in interacting with a remote patient through the medical professionals computer system 814. Furthermore, even in in-person instructional, diagnostic, and therapeutic interactions, a professional may fail to notice various different information-containing expressions, physiological responses, and behaviors or may misinterpret expressions, physiological responses and behaviors exhibited by a student or patient. For example, a teacher may assume that a student gazing out of the window during a lecture is inattentive and failing to concentrate on what the teacher is saying, but, in fact, the student may be carefully listening while processing the information using imagined contexts for examples. In another case, a student sitting erectly and seeming to be attentively listening to a lecture may actually be daydreaming or suffering from attention-robbing anxiety, which the teacher is unable to perceive for Lack of observable manifestations. For all of these reasons, information exchanges are often suboptimally effective due to a lack of precise feedback information from the receiver of information to the information provider, whether the information provider is a human professional or an automated system. Even when sufficient feedback is provided to indicate a problem, the information provider may fail to receive the feedback information or may lack the skill or means to effectively stimulate the information receiver to improve the information exchange. The creation of the currently disclosed methods and systems was motivated by the problems discussed above with reference to FIGS. 8A-C.

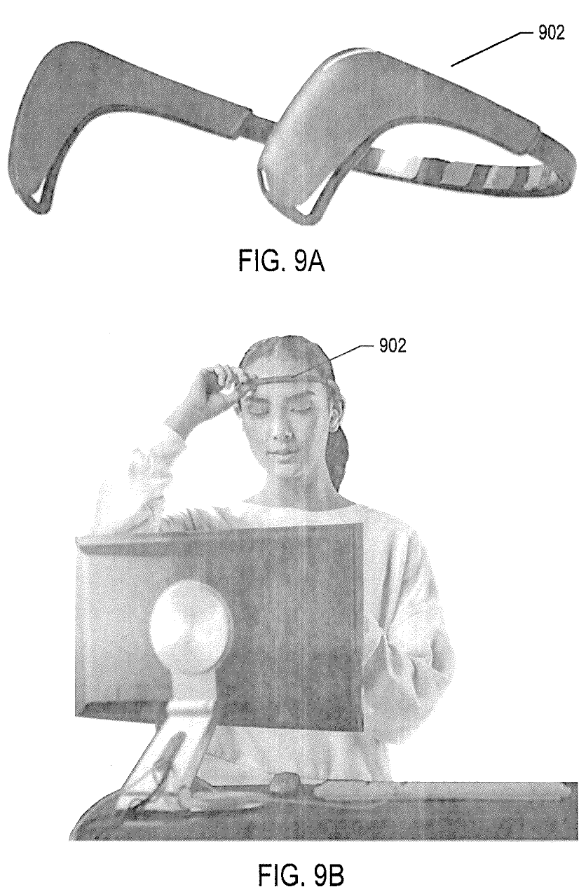

FIG. 9A-B illustrates one of many different types of physiology-sensing computer peripherals that are currently available, Heartbeat-rate and electroencephalography (“EEG”) sensors are incorporated into a headband 902 that communicates heartbeat-rate and EEG signals through Bluetooth radio-frequency wireless communications to a user's mobile device or computer. The EEG signals reflect dynamic voltage fluctuations within the user's brain due to polarization and depolarization of neurons and other electrochemical phenomena, and the intensities and frequencies of the measured voltage fluctuations are correlated with different types of brain activities and mental states. The range of voltage-fluctuation frequencies is divided into frequency bands labeled with Greek characters, including delta, theta, alpha, beta, gamma, and mu frequency bands. Wave-like fluctuations in the intensities, or amplitudes, in each of these frequency bands are indicative of various different types of mental activities and pathologies. For example, alpha waves may be observed when a user is mentally relaxed and may decrease with mental exertion. As shown in FIG. 9B, a student or patient can comfortably wear the headband 902 while participating in a therapeutic, diagnostic, or instructional interaction or in another type of interaction, allowing heartbeat-rate and EEG signals to be communicated to an application running within the user's computer.

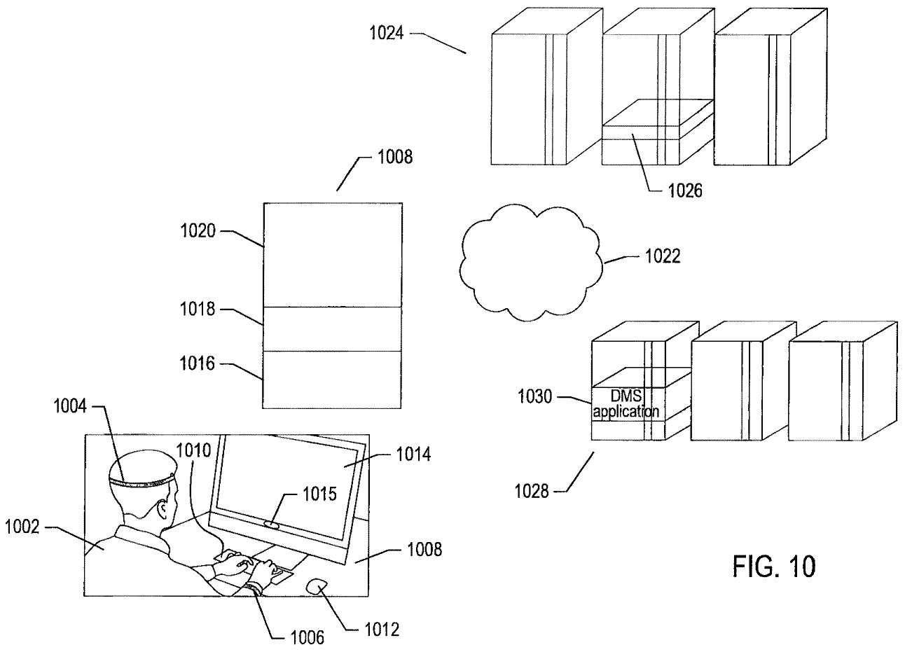

FIG. 10 shows components of and associated with, an example mental-state-monitoring and stimulus-directing system that represents one implementation of the currently disclosed methods and systems. The system is being used by a user 1002. The user is wearing a heartbeat-rate-detecting and EEG-voltage-fluctuation-detecting headband 1004, such as the headband discussed above with reference to FIGS. 9A-B. In addition, in this example, the user is wearing a wristband 1006 in which various physiology sensors are incorporated. These may include a temperature sensor, a pulse oximeter, a blood-pressure sensor, and various additional types of sensors. The user's computer 1008 includes a traditional keyboard 1010, mouse 1012, display screen 1014, and video camera 1015. Internally, the user's computer includes a hardware layer 1016, an operating-system and virtualization layer 1018, and an application layer 1020, as discussed above with reference to FIGS. 1-5A. Cloud icon 1022 represents local-area and wide-area network communications. A first distributed computer system 1024, which may be a data center or cloud-computing facility, includes an information source, such as a Web server, 1026 which provides a stream of audio and video data to the user's computer that represents the information provided to the user as part of an instructional, therapeutic, or diagnostic session or other type of information-provision session. A second distributed computer system includes a distributed, mental-state-monitoring and stimulus-directing application (“DMS application”) 1030 that is a main component of the example mental-state-monitoring and stimulus-directing system (“MM/SD system”).

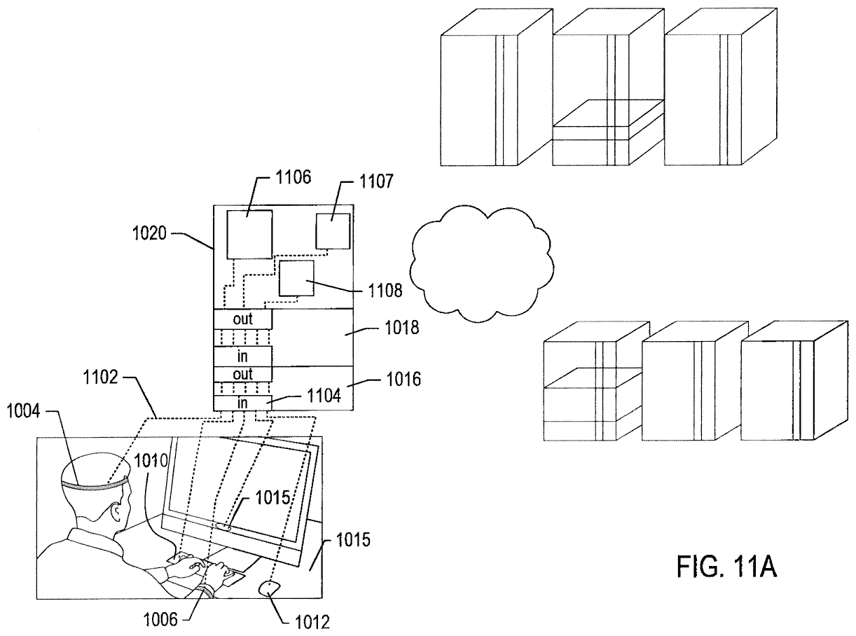

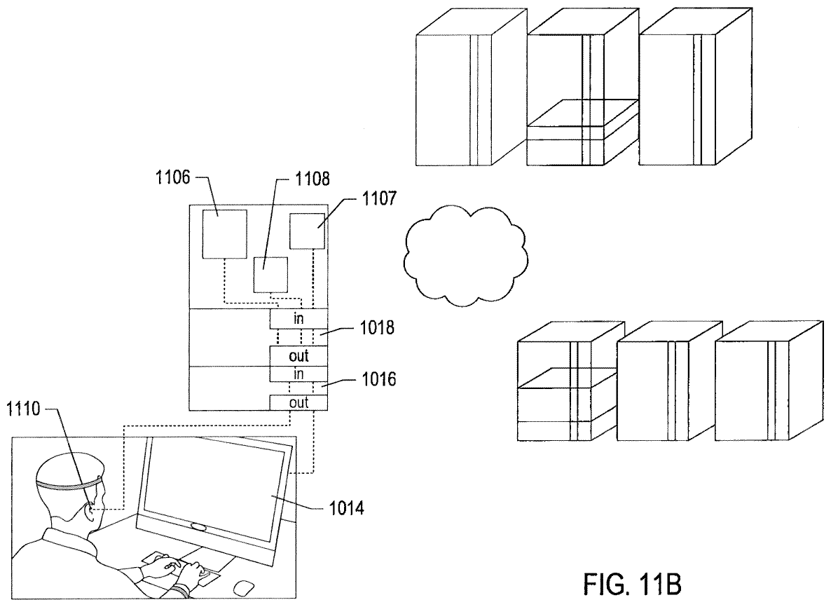

FIGS. 11A-B illustrate data pathways within the user's computer system and between the sensor-containing headband and wristband and the user's computer system in the MM/SD system introduced with reference to FIG. 10. The headband 1004, wristband 1006, and computer-system peripherals 1010, 1012, and 1015 produce data encoded into various types of signals, represented by dashed lines, such as dashed line 1102, which are input to various types of ports and receivers 1104 in the hardware level 1016 of the user's computer. The incoming data is generally temporarily buffered within the hardware layer and then provided to low-level operating-system drivers and other components of the operating-system and virtualization layer 1018. The operating system provides an interface through which various application programs 1106-1108 in the application layer 1020 of the user's computer system access and process the data. As shown in FIG. 11B, the application programs 1106-1108 generate data that is input through an operating-system interface to the operating system 1018, which outputs the data to various transmitters and ports in the hardware layer 1016 for output to a pair of earbuds 110 (another earbud is not visible in FIG. 11B) worn by the user and to the display screen 1014 of the user's computer. There are, of course, many additional interacting components and interfaces within the user's computer system, not shown in FIGS. 10-11B, that contribute to electronic communications between peripheral devices and internal components of the computer system, including application programs running in the application layer of the computer system.

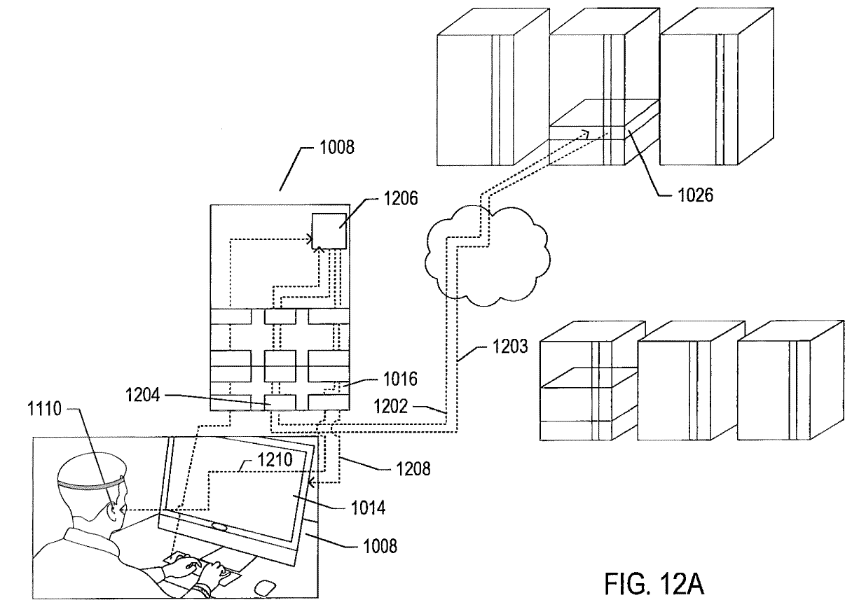

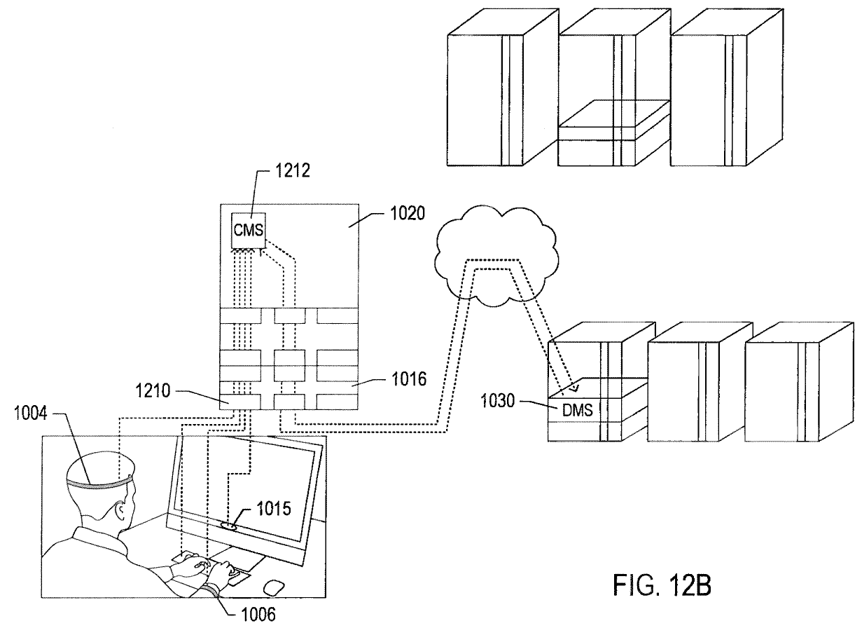

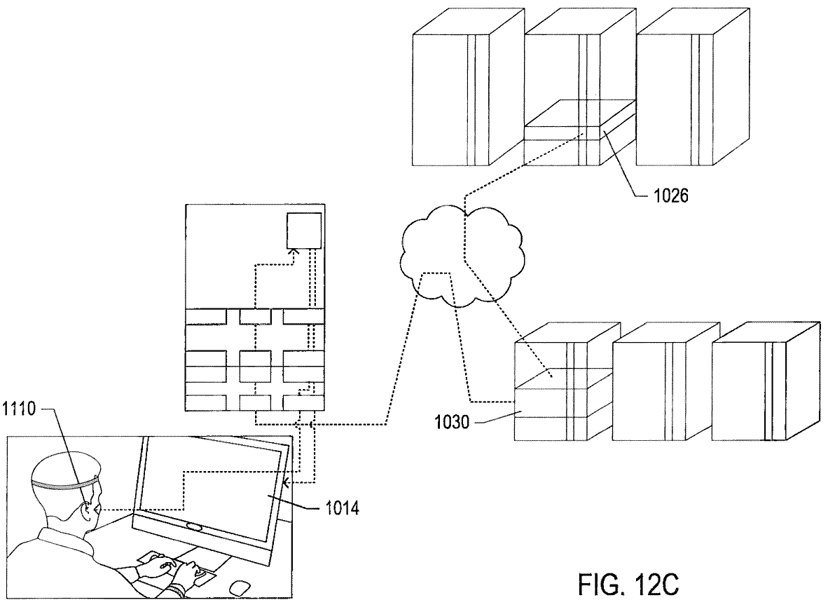

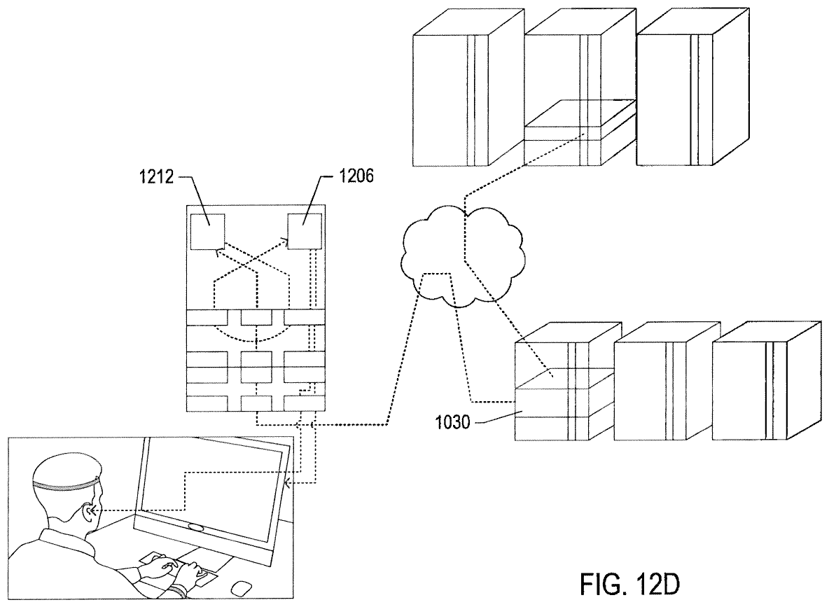

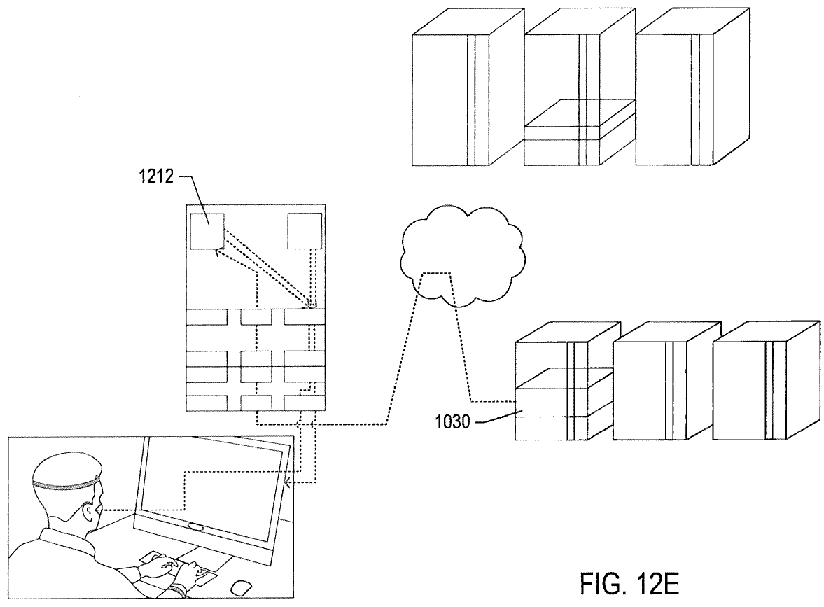

FIGS. 12A-E illustrate operation of the MM/SD system introduced with reference to FIGS. 10-11B. As shown in FIG. 12A, the information source 1026 communicates with the user's computer system 1008 via network communications represented by dashed arrows 1202-1203. Network packets transmitted by the information source to the user's computer are received by a network interface card 1204 within the hardware layer 1016 and are internally communicated through the operating system to a client-side application or applications 1206 that output information received from the information source to the display screen 1014 and earbuds 1110 worn by the user. The client-side application or applications 1206 may be a web browser, a client-side application associated with the information source, or another application that receives information from the information source and presents the information to the user via the earbuds 1110 and the display screen 1014. Audio and video information extracted from network packets is streamed, by the application or applications 1206 to the earbuds and display screen, through operating-system interfaces and device drivers, to a sound card and GPU within the hardware layer 1016 that then output audio and visual signals to the earbuds and display screen, as represented by dashed arrows 1208-1210. In certain cases, received audio and visual information may be more directly transformed into output audio and visual signals at levels below the application level. The user launches presentation of information provided by the information source via commands input through the keyboard and mouse and transferred to the application or applications 1206.

FIG. 12B shows collection of various types of monitoring signals by the DMS application 1030. While the user is viewing and listening to information provided by the information source, as discussed above with reference to FIG. 12A, various types of monitoring data are collected from the sensor-containing headband 1004, wristband 1006, video camera 1015, and potentially other monitoring-signal sources, including a microphone and other types of physiology sensors. The monitoring data are received by various ports and receivers 1210 in the hardware layer 1016 of the user's computer system and transferred through the operating system to a client-side mental-state-monitoring and stimulus-directing application (“CMS application”) 1212 running in the application layer 1020 of the user's computer system. The various monitoring data are continuously packaged, into network packets, and transferred by the CMS application to the DMS application 1030 via network communications. Each different type of monitoring data may be logically considered to be a different data channel, with some data channels including multiple subchannels. The monitoring data multiplexed into network packets by the CMS application is extracted and demultiplexed from the network packets by the DMS application. The CMS application may also receive input from the user's keyboard and mouse representing commands and information, such as commands to begin and end monitoring and requests for registration, login, and logout, in certain implementations.

One noteworthy feature of the MM/SD system is that the CMS and DMS applications can be independent of the information source. Mental-state monitoring and stimulus direction can therefore be carried out independently of the type of information and type of information-provision session that is being provided to the user by the information source and application or applications 1206. In certain embodiments of the MM/SD system, user input to the CMS application can provide indications of the information source and information-provision session to the DMS application, allowing the DMS application to specifically interpret received monitoring data in the context of the information source and information-provision session currently viewed and listened to by the user. Thus, independence of the MM/SD system from the information source is not required, but can facilitate wider applicability and efficiency, in certain cases. In other cases, the MM/SD system may be coupled with the information source to varying degrees in order to provide more accurate monitoring and more precise stimulation.

FIGS. 12C-E illustrate various different ways in which the DMS application provides stimuli to the user as the DMS application processes monitoring data and detects various types of conditions and problems that the DMS application determines to be addressable by stimulus provision. As shown in FIG. 12C, in certain implementations, the DMS application 1030 can act as an intermediary in data transmission from the information source 1026 to the user's computer system. Rather than directly transmitting information to the user's computer system, as shown in FIG. 12A, the information is redirected to the DMS application 1030 which can then modify the information before forwarding the information to the user's computer system. The modifications to the information received from the information source can introduce various types of stimuli for provision to the user via peripheral devices associated with the user's computer system, including the display screen 1014 and earbuds 1110. As one example, the level of display-screen illumination may be varied at a peak EEG alpha-band frequency, which may provide an attention-focusing stimulus to the user. Other types of stimulus may involve altering the audio signal, superimposing various different sounds and visual effects over the audio and visual portions of the information display, adding frames to the visual-display information, pausing and restarting display of the information, and making many other types of modifications to the information received from the information source prior to forwarding the information to the user's computer. Stimuli ma also comprise videos, text, broadcast audio, and essentially anything that can be provided by a users computer system to the user in order to stimulate changes to the user's mental state.

Alternatively, as shown in FIG. 12D, the DMS application 1030 may instead send stimulus directives through the network to the CMS application 1212 which then uses interprocess communications within the user's computer system to forward the directives to the application or applications 1206. The application or applications 1206 then modify the information received from the information source, as discussed above with reference to FIG. 12C, before transmitting the information to the operating system and hardware-layer components for rendering for display and broadcast to the user. In yet another alternative stimulus-generation method illustrated in FIG. 12E, the DMS application 1030 may transmit stimulus directives to the CMS application 1212 which then carries out the directives by directly transmitting display and broadcast commands to an operating-system interface. These commands may direct the operating system to modulate the display-screen illumination level or otherwise introduce various types of stimulus signals into the information display and broadcast. Various combinations of the three types of stimulus generation discussed above with reference to FIGS. 12C-E may be employed concurrently by the DMS application. Additional methods for generating stimuli are also possible.

In alternative implementations, the DMS application may be combined with the CMS application within the user's computer system, when the user's computer system has sufficient computational bandwidth to support the DMS-application Functionality for a single user. As mentioned above, many different types of monitoring data can be collected and provided to the DMS application and many different types of stimuli can be generated according to directives supplied by the DMS application for presentation to the user. The MM/SD system provides feedback to a user while the user consumes information from an information source just as a teacher, physician, or therapist may attempt to provide feedback to a student or patient in order to optimize information communication. In certain ways, the MM/SD system may have advantages with respect to a human professional, including an ability to carry out precise, real-time monitoring of many different types of monitoring data, an ability to continuously determine a user's mental state by continuously processing the monitoring data, and an ability to generate a wide variety of different types of stimulus for the user in real time, without significant lag times. Furthermore, the MM/SD system is able to monitor mental states and provide various types of stimulus feedback in many different types of information-provision contexts in which there is no human professional involved in the information-provision session. Because of the MM/SD system's ability to continuously determine the user's mental state, the MM/SD system is able to avoid providing deleterious or unsafe stimuli to a user. As one example, certain types of stimulus can provoke epileptic seizures, anxiety, or depression, and the MM/SD system can detect mental states prone to these types of negative consequences and avoid the associated types of stimulus.

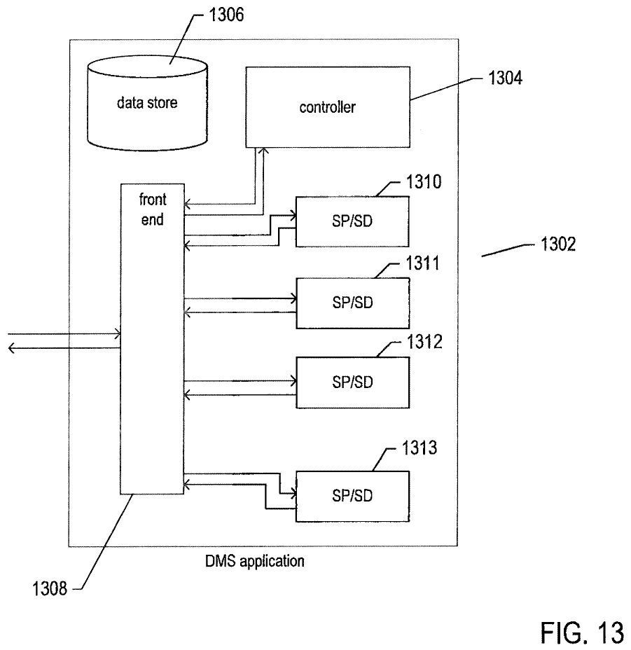

FIGS. 13-20 illustrate one implementation of the MM/SD system. FIG. 13 shows a high-level block diagram of the DMS application. The DIS application 1302 includes a controller 1304, data stored in a data store 1306, a front end 1308, and multiple signal-processor and stimulus-director components (“SP/SD components”) 1310-1313. In many implementations, the controller, front end, and SP/SD components are each implemented in a different virtual machine or set of virtual machines within a cloud-computing facility and the data store may represent a virtual data-storage appliance, provided to virtual machines of a cloud-computing facility, that provide data-storage resources of one or more physical data-storage devices. The controller is responsible for receiving and responding to various types of user commands and system events. For example, the controller may register new users, carry out redirection of information transmitted from an information source, as discussed above with reference to FIG. 12C, carry out user login and logout operations, perform many different types of additional administrative functions, initialize communications connections to CMS applications running within user computer systems and launching SP/SD components, and carry out many additional types of tasks. The front end receives network messages from different user computers and other remote entities and forwards them to either the controller or to SP/SD components. The front end receives network messages from the controller and SP/SD components and forwards them to the appropriate remote CMS applications running within user computer systems and other remote entities. In certain implementations, the CMS application, once launched on a user's computer system, automatically begins to monitor the user's mental state and provide stimuli, without needing to be commanded to do so. In another implementation, the user wishes commands to the CMS application in order to start and stop monitoring. In certain implementations, users are required to register with the MM/SD prior to monitoring and stimulus provision while, in other implementations, users are required to initially register and then to login and logout of the MM/SD for monitoring and stimulus-provision sessions.

FIG. 4 illustrates one implementation of an SP/SD component. An SP/SD component receives network messages containing monitoring data received by the DMS applications from a user computer, processes the received monitoring data in order to detect or determine various different types of mental-state attribute values, generates mental-state-vector representations of the user's mental state at successive points in time, and then continuously or periodically uses the mental-state vector representations to determine whether or not one or more stimuli should be provided to the user and, if so, constructs stimulus-directive network messages for transmission back to the CMS application in the user's computer system. In other words, an SP/SD component is responsible for real-time monitoring of a user and real-time generation of stimulus directives for provision to the user. In the currently described DMS application, an SP/SD component is launched by the controller for each user that has requested monitoring. In alternative implementations, users may be assigned to user classes and an SP SD component implemented to concurrently monitor multiple different users of the user class is launched by the controller to monitor and generate stimuli for multiple users of the user class corresponding to the SP/SD component. Additional types of implementations are possible.

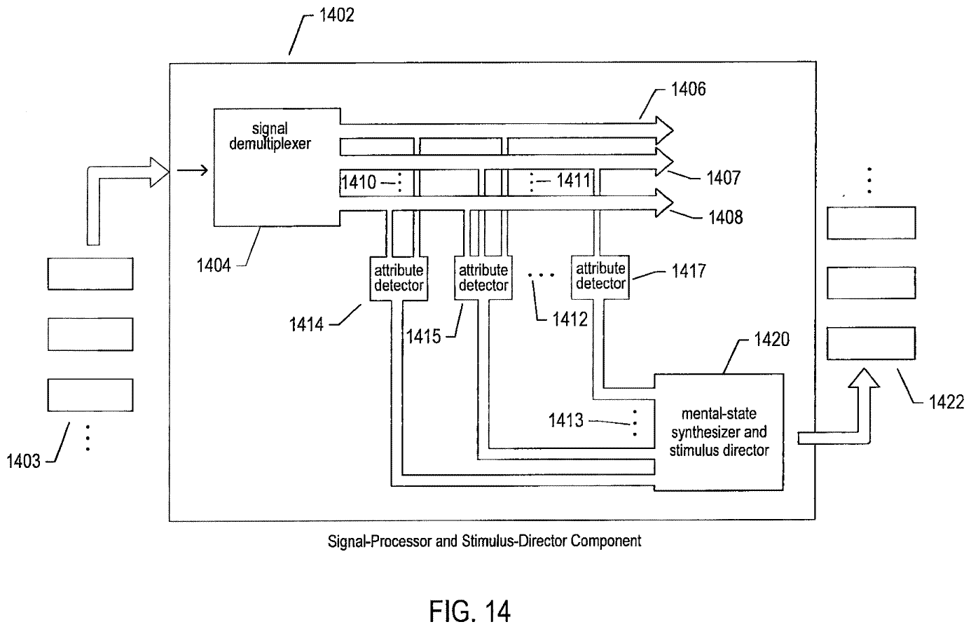

In FIG. 14, the monitoring-data-containing network messages 1404 are shown being input to the left side of the SP/SD component 1402. The incoming network messages are processed by a signal demultiplexer 1404 which extracts monitoring data for each monitoring-data channel from the input network messages and outputs the monitoring data to multiple monitoring-data output buses 1406-1408. Ellipses 1410-1413 are used to indicate that there may be additional monitoring-data channels and attribute detectors, discussed below, along with corresponding SP/SD components. Of course, a particular SP/SD component implementation may include only one or two monitoring-data channels rather than the three or more monitoring-data channels shown in FIG. 14. A set of attribute detectors 1414-1417 receive monitoring data from one or more of the monitoring-data output buses and process the monitoring data in order to output attribute indications to a mental-state synthesizer and stimulus director 1420. The mental-state synthesizer and stimulus director generates mental-state vectors representing the current mental state of the user, inferred from the monitoring data received by the SP/SD component, and then uses the generated mental-state vectors to determine whether or not a stimulus or stimuli should be provided to the user and, if so, generates stimulus directives packaged into network messages 1422 that are output to the DMS-application front and (1308 in FIG. 13) for transmission to the user's computer system.

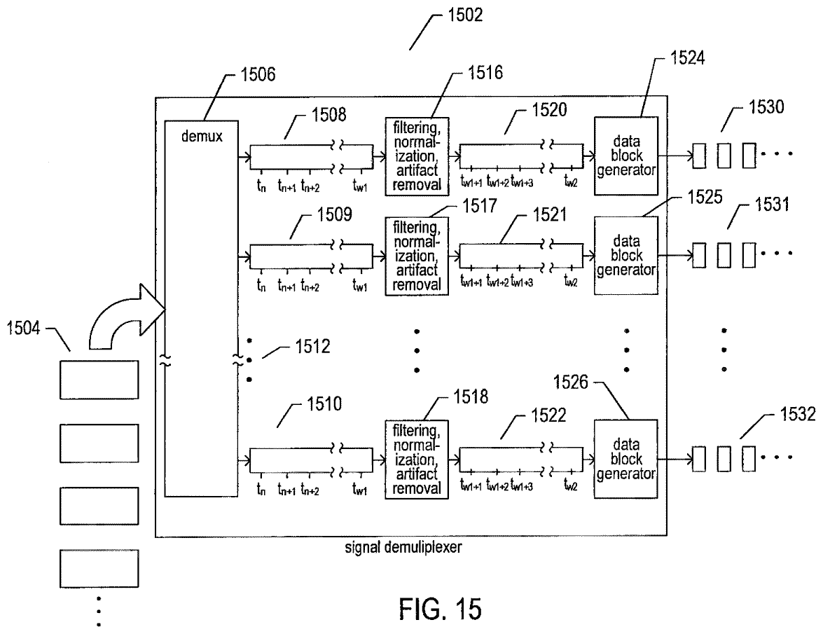

FIG. 15 illustrates the signal-demultiplexer component (1404 in FIG. 14) of an SP/SD component of the DMS application. The signal-demultiplexer component 1502 receives network messages containing monitoring data 1504 from the front end of the DMS application (1308 in FIG. 13). The network messages are input to a demux component 1506 that extracts the monitoring data from the network messages into separate monitoring-data channels 1508-1510, with ellipsis 1512 indicating that there may be additional monitoring-data channels. Each monitoring-data channel corresponds to a different type of monitoring data, such as heartbeat-rate data, EEG data, video-camera data, physiology-sensor data generated by a sensor within the wristband 1006, and any other type of monitoring data transmitted by the CMS application to the DMS application. The monitoring-data channels may be implemented, as one example, by first-in/first-out queues or circular queues. The raw data is processed by filtering, normalization, and artifact-removal components 1516-1518. Each different monitoring-data channel is processed by a filtering, normalization, and artifact-removal component specific to the type of data stored in the monitoring-data channel. These components remove noise, filter out unwanted frequencies from certain types of data, remove outlier data due to a variety of data-corruption events and phenomena, and carry out other types of processing to maximize the signal-to-noise ratio associated with the data. Normalization is carried out in order to standardize data across subchannels and data-acquisition times. Processed data is output to processed-data channels 1520-1522. The processed data is then divided into blocks of data by data-block-generator components 1524-1526. These components produce a stream of data blocks 1530-1532 that are output to the monitoring-data output buses (1406-1408 in FIG. 14). In different implementations, the order of operations carried out by the filtering, normalization, and artifact removal components and the data-block-generator components may be varied and the positions of the filtering, normalization, and artifact removal components and the data-block-generator components may be interchanged for particular monitoring-data channels.

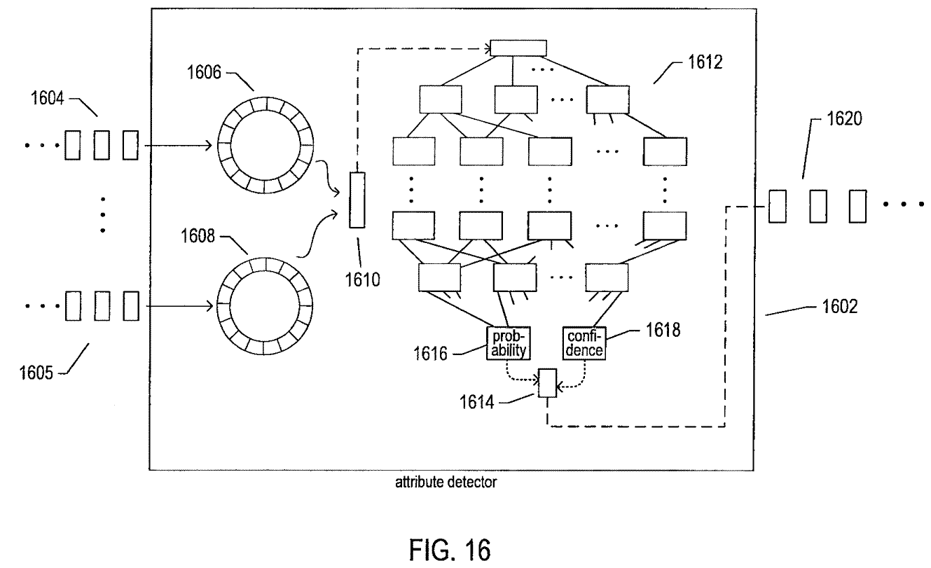

FIG. 16 illustrates an attribute-detector component of an SP/SD component of the DMS application. An attribute detector 1602 receives monitoring-data blocks 1604 and 1605 output, by data-block-generator components (1524-1526 in FIG. 15) of the signal demultiplexer (1502 in FIG. 15), to the monitoring-data output buses (1406-1408 in FIG. 14). The monitoring-data blocks are queued to circular input queues 1606 and 1608. A given attribute detector may receive data blocks from one or more monitoring-data output buses. Monitoring-data blocks are removed from the queues, as they become available, to construct input-data vectors 1610 that are input to a neural network 1612, or another type of machine-learning analyzer, for analysis. For each input-data vector, the neural network or other type of machine-learning analyzer produces an attribute indicator 1614 that includes an indication of the probability of the occurrence of the attribute 1616 and an indication of the confidence 1618 associated with the probability indication. A softmax function may be applied to outputs from the neural network to generate a probability. For example, an attribute detector that detects inattentiveness outputs attribute indication that includes an estimate of the probability that the user is currently inattentive along with a confidence value associated with the estimate. In one approach, there may be a set of related attributes for different severity levels of inattentiveness, with indications of the probability of each level of inattentiveness and an associated confidence returned for each level of inattentiveness. Alternatively, a single inattentiveness attribute may be determined from the monitoring data as a real-number attribute value indicating the current degree or severity of inattentiveness. Many other types of outputs can be generated by neural networks or other machine-learning entities incorporated in attribute detectors, with neural-network outputs additionally processed by various functions, such as softmax, to produce the attribute-indication outputs. Machine-learning systems may include neural networks, neural networks combined with additional functionalities, such as softmax, rule-based systems, decision trees, and many other types of systems that can be trained from training data to produce desired results. The attribute indication is associated with a short time interval that spans the timestamps associated with the monitoring-data blocks used to generate the input vector. However, in certain implementations, generation of the input vector may also consider additional monitoring-data blocks stored in the circular queues and running statistical metrics in order to provide probability indications based on a wider data context. The attribute indications are output 1620 by the attribute detector for input to the mental-state-synthesizer-and-stimulus-director component (1420 in FIG. 14, of the SP SD component of the DMS application.

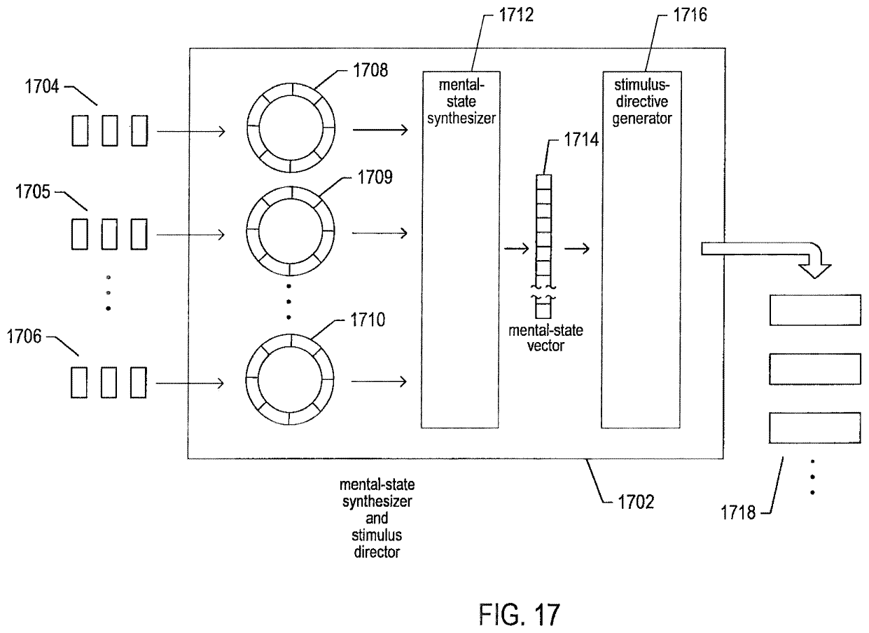

FIG. 17 illustrates the mental-state-synthesizer and stimulus-director component (1420 in FIG. 14) of an SP/SD component of the DMS application. The mental-state-synthesizer and stimulus-director component 1702 receives attribute indications 1704-1706 from attribute detectors (1602 in FIG. 16) of an SP/SD component of the DMS application. These attribute indications are stored in circular input queues 1708-1710. Attribute indications are then dequeued and processed by a mental-state synthesizer 1712 and used to generate a mental-state vector 1714 for a period of time that spans the time stamps of the monitoring-data blocks used to generate the attribute indications. The mental-state vectors are input to a stimulus-directive generator 1716 which determines whether or not the user's current mental state, as indicated by the mental-state vector, warrants provision of one or more stimuli to the user. In the case that one or more stimuli are warranted, the stimulus-directive generator determines one or more stimuli for provision to the user along with characteristics of the stimuli, including the time span over which the stimuli should be provided, the intensity of stimulation, and other characteristics and attributes of the desired stimuli and packages directives, or commands, for generating the stimuli into network messages 1718 that are returned to the front end (1308 in FIG. 13) of the DMS application for transmission to the user's computer. The stimulus-directive generator may be implemented as one or more neural networks or as one or more other types of machine-learning-based analyzers.

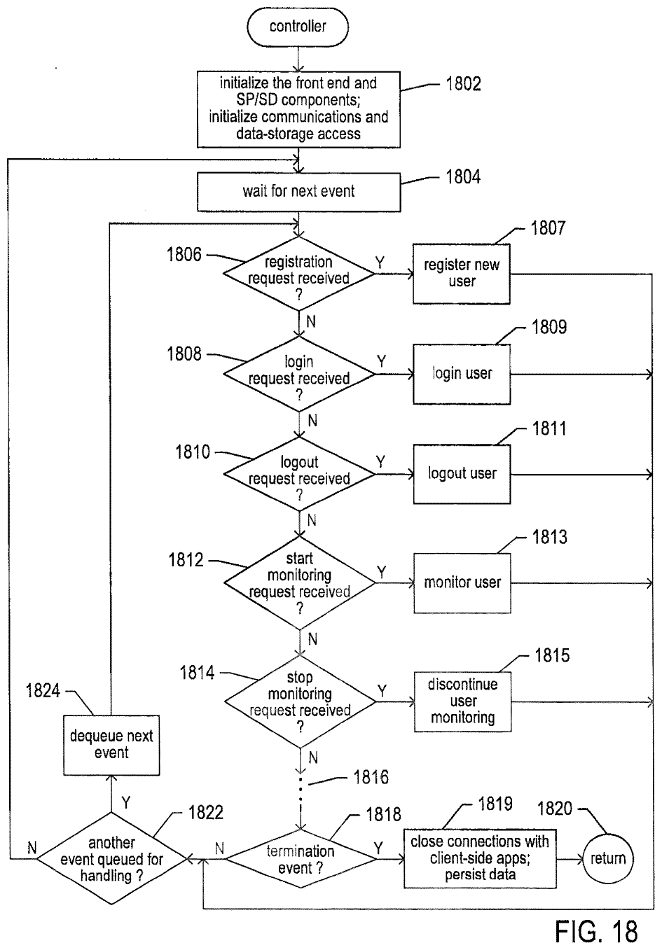

FIG. 18 provides a control-flow diagram that illustrates operation of the controller (1304 in FIG. 13) of the DMS application. The controller is logically implemented as an event-handling loop. In step 1802, the controller, upon launching, initializes the various additional components of the DSM application as well as communications and data-storage access. Then, in step 1804, the controller waits for the occurrence of a next event. When the next event is reception of a registration request from a new user, as determined in step 1806, a register-new-user routine is called, in step 1807. Otherwise, when the next event is reception of a login request from a user, as determined in step 1808, a login-user routine is called in step 1809. Otherwise, when the next event is a logout request received from a user, as determined in step 1810, a logout-user routine is called step 1811. Similarly, start-monitoring and stop-monitoring requests are detected and handled in steps 1812-1815. Ellipsis 1816 indicates that many additional types of events may be handled by the controller. The occurrence of a termination event, detected in step 1818 results in the controller closing communications connections and persisting data, and step 1819, before execution of the vent loop terminates in step 1820. When another event has been queued for handling, as detected in step 1822, a next event is dequeued, in step 1824, and control returns to step 1806 to handle the next event. Otherwise, control returns to step 1804 where the controller waits for the occurrence of a next event,

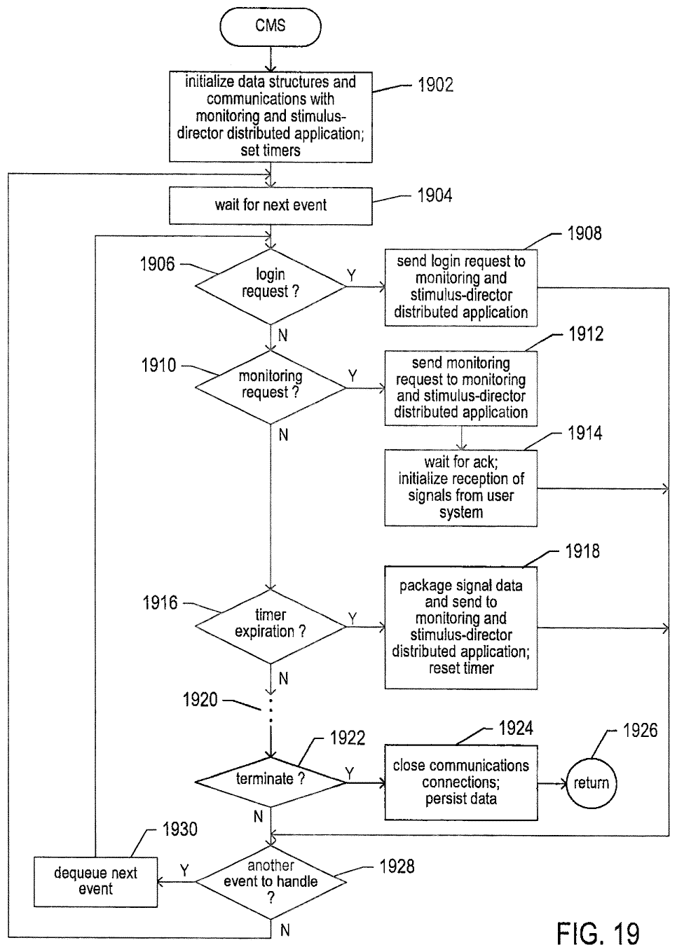

FIG. 19 provides a control-flow diagram that illustrates operation of the CMS application (1212 in FIG. 12B). In step 1902, the CMS initializes data structures and communications with the DMS application and sets various timers, including a signal-reporting timer. Then, in step 1904, the CMS waits for the occurrence of a next event. When the next event is reception of a login request from the use, as determined in step 1906, the CMS sends a login request to the DMS application, in step 1908. In addition, the CMS may set a timer to detect failure of the DMS to respond to the login request. When the next event is a monitoring request, as determined in step 1910, the CMS sends a monitoring request to the DMS application, in step 1912, waits for an acknowledgment from the DMS application, and then initializes reception of monitoring signals from the user's computer system, in step 1914. When the next occurring event is a timer expiration, as determined in step 1916, the CMS packages monitoring data received since the last monitoring-data message was sent to the DMS into a monitoring-data network message and sends the monitoring-data network message to the DMS application, in step 1918. Ellipsis 1920 indicates that many other events are handled by the CMS. When a termination event occurs, as determined in step 1922, the CMS closes communications connections and persists data, in step 1924, before terminating execution, in step 1926. When another event has been queued for handling, as determined in step 1928, a next event is dequeued, in step 1930, and control returns to step 1906 for handling of the next event. Otherwise, control returns to step 1904, where the CMS waits for the occurrence of a next event.

### Neural Networks

FIG. 20 illustrates fundamental components of a feed-forward neural network. Expressions 2002 mathematically represent ideal operation of a neural network as a function ƒ(x). The function receives an input vector x and outputs a corresponding output vector y 1103. For example, an input vector may be a digital image represented by a two-dimensional array of pixel values in an electronic document or may be an ordered set of numeric or alphanumeric values. Similarly, the output vector may be, for example, an altered digital image, an ordered set of one or more numeric or alphanumeric values, an electronic document, or one or more numeric values. The initial expression of expressions 2002 represents the ideal operation of the neural network. In other words, the output vector y represents the ideal, or desired, output for corresponding input vector x. However, in actual operation, a physically implemented neural network \hat{ƒ}(x), as represented by the second expression of expressions 2002, returns a physically generated output vector ŷ that may differ from the ideal or desired output vector y. An output vector produced by the physically implemented neural network is associated with an error or loss value. A common error or loss value is the square of the distance between the two points represented by the ideal output vector y and the output vector produced by the neural network ŷ. The distance between the two points represented by the ideal output vector and the output vector produced by the neural network, with optional scaling, may also be used as the error or loss. A neural network is trained using a training dataset comprising input-vector/ideal-output-vector pairs, generally obtained by human or human-assisted assignment of ideal-output vectors to selected input vectors. The ideal-output vectors in the training dataset are often referred to as “labels.” During training, the error associated with each output vector, produced by the neural network in response to input to the neural network of a training-dataset input vector, is used to adjust internal weights within the neural network in order to minimize the error or loss. Thus, the accuracy and reliability of a trained neural network is highly dependent on the accuracy and completeness of the training dataset.

As shown in the middle portion 2006 of FIG. 20, a feed-forward neural network generally consists of layers of nodes, including an input layer 2008, an output layer 2010, and one or more hidden layers 2012. These layers can be numerically labeled 1, 2, 3, . . . , L−1, L, as shown in FIG. 20. In genera the input layer contains a node for each element of the input vector and the output layer contains one node for each element of the output vector. The input layer and/or output layer may each have one or more nodes. In the following discussion, the nodes of a first level with a numeric label lower in value than that of a second layer are referred to as being higher-level nodes with respect to the nodes of the second layer. The input-layer nodes are thus the highest-level nodes. The nodes are interconnected to form a graph, as indicated by line segments, such as line segment 2014.

The lower portion of FIG. 20 (2020 in FIG. 20) illustrates a feed-forward neural-network node. The neural-network node 2022 receives inputs 2024-2027 from one or more next-higher-level nodes and generates an output 2028 that is distributed to one or more next-lower-level nodes 2030. The inputs and outputs are referred to as “activations,” represented by superscripted-and-subscripted symbols “a” in FIG. 20, such as the activation symbol 2024. An input component 2036 within a node collects the input activations and generates a weighted sum of these input activations to which a weighted internal activation a₀ is added. An activation component 2038 within the node is represented by a function g( ), referred to as an “activation function,” that is used in an output component 2040 of the node to generate the output activation of the node based on the input collected by the input component 2036. The neural-network node 2022 represents a generic hidden-layer node. Input-layer nodes lack the input component 2036 and each receive a single input value representing an element of an input vector. Output-component nodes output a single value representing an element of the output vector. The values of the weights used to generate the cumulative input by the input component 2036 are determined by training, as previously mentioned. In general, the input, outputs, and activation function are predetermined and constant, although, in certain types of neural networks, these ma % also be at least partly adjustable parameters. In FIG. 20, three different possible activation functions are indicated by expressions 2042-2044. The first expression is a binary activation function and the third expression represents a sigmoidal relationship between input and output that is commonly used in neural networks and other types of machine-learning systems, both functions producing an activation in the range [0, 1]. The second function is also sigmoidal, but produces an activation in the range [−1, 1].

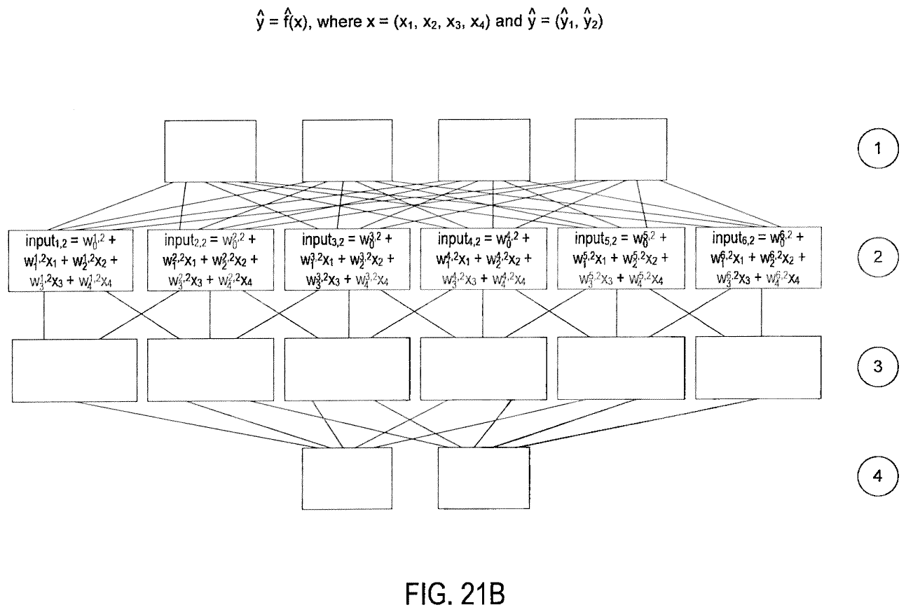

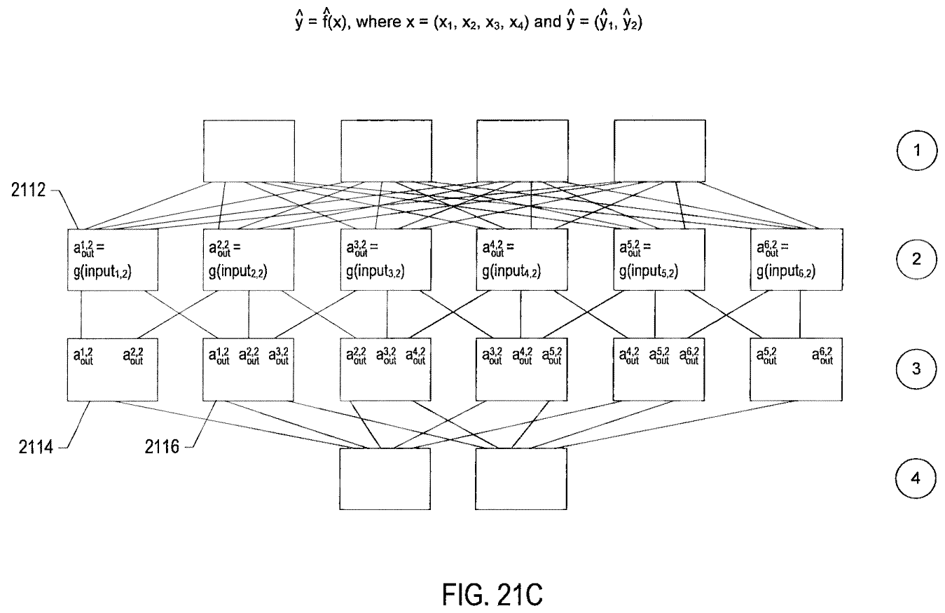

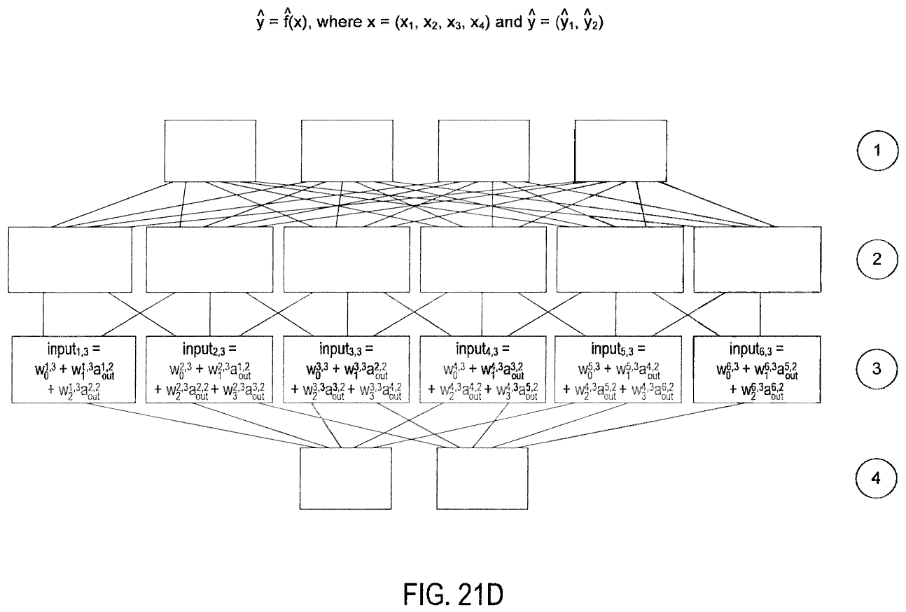

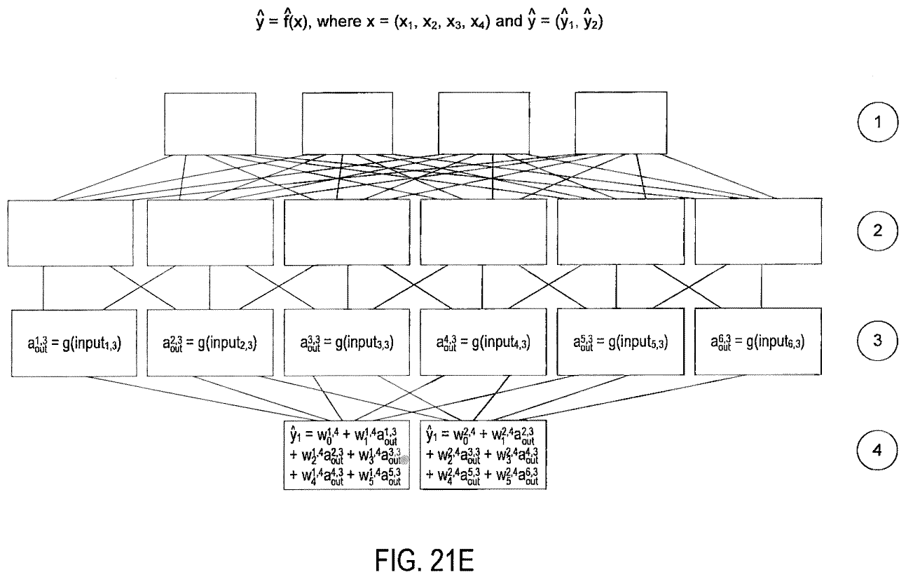

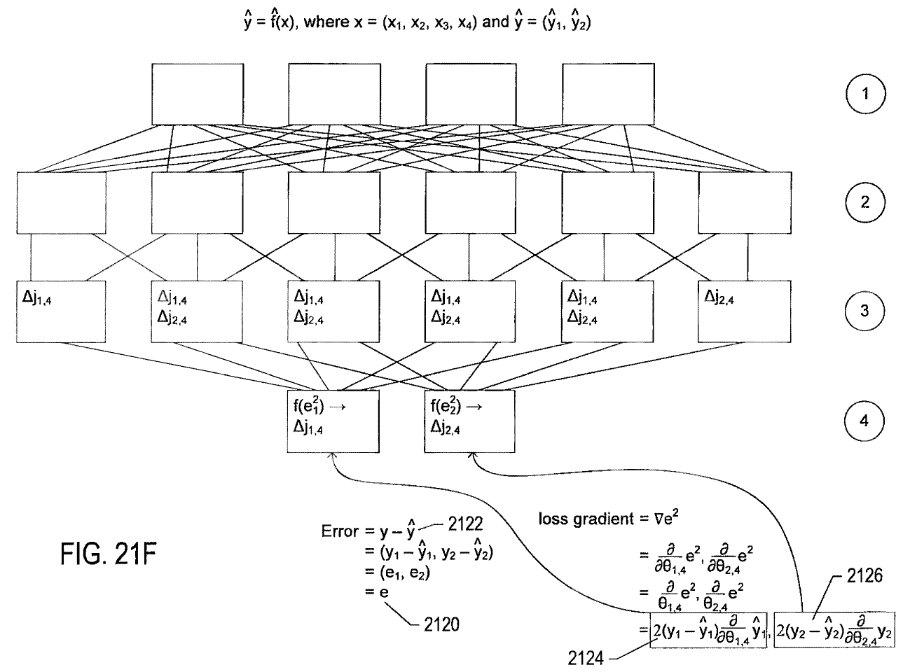

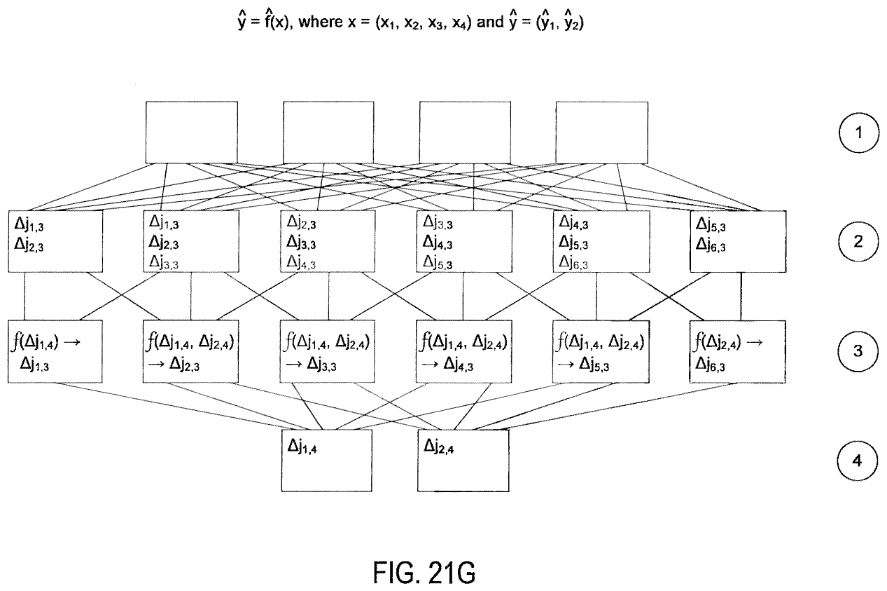

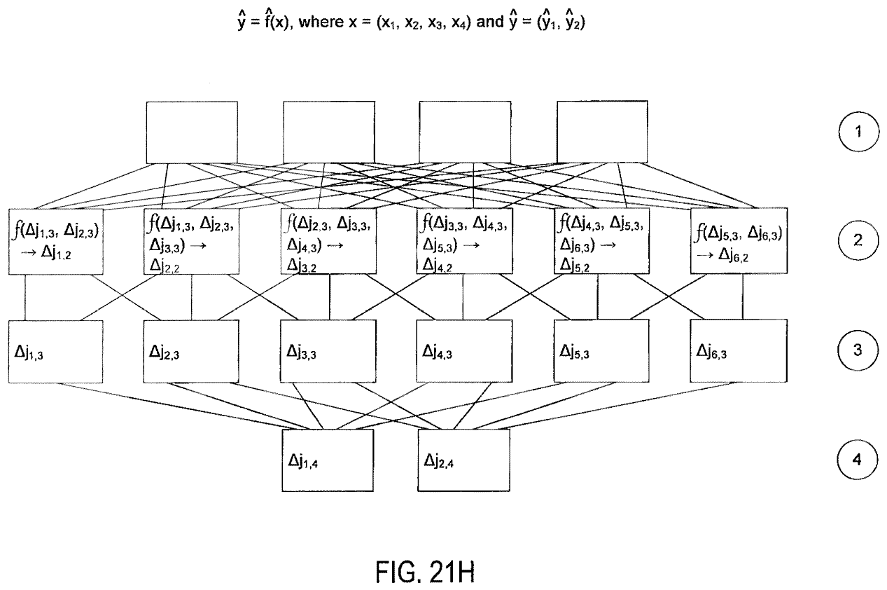

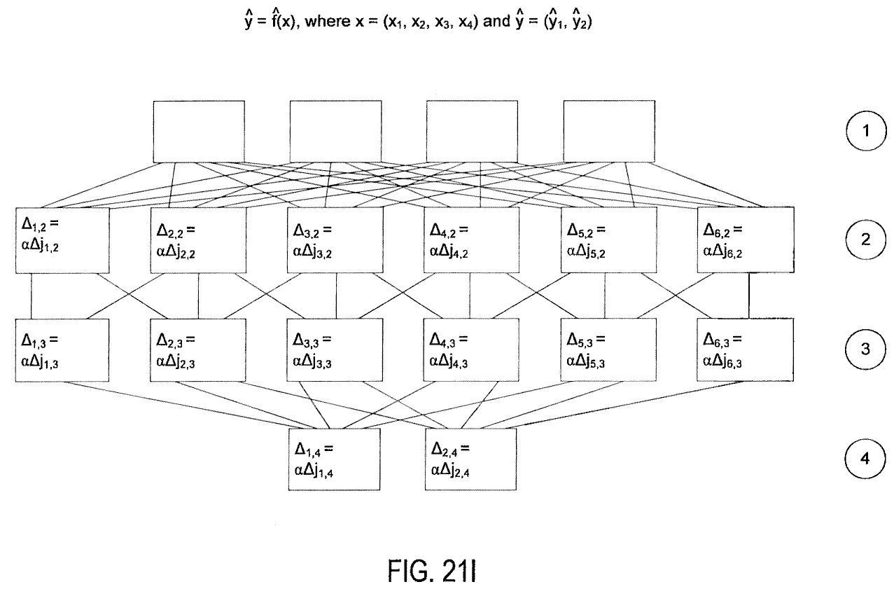

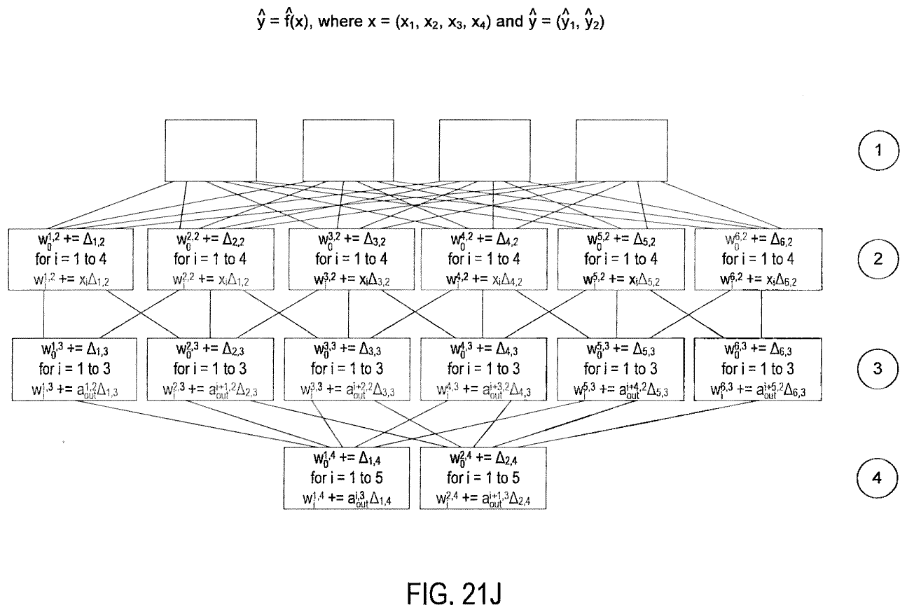

FIGS. 21A-J illustrate operation of a very small, example neural network. The example neural network has four input nodes in a first layer 2102, six nodes in a first hidden layer 2104 six nodes in a second hidden layer 2106, and two output nodes 2108. As shown in FIG. 21A, the four elements of the input vector x 2110 are each input to one of the four input nodes which then output these input values to the nodes of the first-hidden layer to which they are connected. In the example neural network, each input node is connected to all of the nodes in the first hidden layer. As a result, each node in the first hidden layer has received the four input-vector elements, as indicated in FIG. 21A. As shown in FIG. 21B, each of the first-hidden-layer nodes computes a weighted-sum input according to the expression contained in the input components (2036 in FIG. 20) of the first hidden-layer nodes. Note that, although each first-hidden-layer node receives the same four input-vector elements, the weighted-sum input computed by each first-hidden-layer node is generally different from the weighted-sum inputs computed by the other first-hidden-layer nodes, since each first-hidden-layer node generally uses a set of weights unique to the first-hidden-layer node. As shown in FIG. 21C, the activation component (2038 in FIG. 20) of each of the first-hidden-layer nodes next computes an activation and then outputs the computed activation to each of the second-hidden-layer nodes to which the first-hidden-layer node is connected. Thus, for example, the first-hidden-layer node 2112 computes activation aₒᵤₜ^{1,2} using the activation function and outputs this activation to second-hidden-layer nodes 2114 and 2116. As shown in FIG. 21D, the input components (2036 in FIG. 20) of the second-hidden-layer nodes compute weighted-sum inputs from the activations received from the first-hidden-layer nodes to which they are connected and then, as shown in FIG. 21E, compute activations from the weighted-sum inputs and output the activations to the output-layer nodes to which they are connected. The output-layer nodes compute weighted sums of the inputs and then output those weighted sums as elements of the output vector.

FIG. 21F illustrates backpropagation of an error computed for an output vector. Backpropagation of a loss in the reverse direction through the neural network results in a change in some or all of the neural-network-node weights and is the mechanism by which a neural network is trained. The error vector e 2120 is computed as the difference between the desired output vector y and the output vector ŷ (2122 in FIG. 21F) produced by the neural network in response to input of the vector x. The output-layer nodes each receive a squared element of the error vector and compute a component of a gradient of the squared length of the error vector with respect to the parameters θ the neural-network, which are the weights. Thus, in the current example, the squared length of the error vector e is equal to |e|² or e₁²+e₂², and the loss gradient is equal to:

$${∇}_{0} ( \text{?} + {e}_{2}^{2} ) = \frac{\partial }{\partial θ} \text{?} ,$$

$$\frac{\partial }{\partial θ} \text{?} .$$

$$\text{?} \text{indicates text missing or illegible when filed}$$

Since each output-layer neural-network node represents one dimension of the multi-dimensional output, each output-layer neural-network node receives one term of the squared distance of the error vector and computes the partial differential of that term with respect to the parameters, or weights, of the output-layer neural-network node. Thus, the first output-layer neural-network node receives e₁² and computes

$$\frac{\partial }{{\partial }_{.} {θ}_{1.4}} {e}_{1}^{2} ,$$

where the subscript 1,4 indicates parameters for the first node of the fourth, or output, layer. The output-layer neural-network nodes then compute this partial derivative, as indicated by expressions 2124 and 2126 in FIG. 21F. The computations are discussed later. However, to follow the backpropagation diagrammatically, each node of the output layer receives a term of the squared length of the error vector which is input to a function that returns a weight adjustment Δ_{J}. As shown in FIG. 21F, the weight adjustment computed by each of the output nodes is back propagated upward to the second-hidden-layer nodes to which the output node is connected. Next, as shown in FIG. 21G, each of the second-hidden-layer nodes computes a weight adjustment Δ_{J} from the weight adjustments received from the output-layer nodes and propagates the computed weight adjustments upward in the neural network to the first-hidden-layer nodes to which the second-hidden-layer node is connected. Finally, as shown in FIG. 21H, the first-hidden-layer nodes computes weight adjustments based on the weight adjustments received from the second-hidden-layer nodes. These weight adjustments are not, however, back propagated further upward in the neural network since the input-layer nodes do not compute weighted sums of input activations, instead each receiving only a single element of the input vector x.

In a next logical step, shown in FIG. 21I, the computed weight adjustments are multiplied by a learning constant α to produce final weight adjustments Δ for each node in the neural network. In general, each final weight adjustment is specific and unique for each neural-network node, since each weight adjustment is computed based on a node's weights and the weights of lower-level nodes connected to a node via a path in the neural network. The logical step shown in FIG. 21I is not, in practice, a separate discrete step since the final weight adjustments can be computed immediately following computation of the initial weight adjustment by each node. Similarly, as shown in FIG. 21J, in a final logical step, each node adjusts its weights using the computed final weight adjustment for the node. Again, this final logical step is, in practice, not a discrete separate step since a node can adjust its weights as soon as the final weight adjustment for the node is computed. It should be noted that the weight adjustment made by each node involves both the final weight adjustment computed by the node as well as the inputs received by the node during computation of the output vector ŷ from which the error vector e was computed, as discussed above with reference to FIG. 21F. The weight adjustment carried out by each node shift the weights in each node toward producing an output that, together with the outputs produced by all the other nodes following weight adjustment, results in decreasing the distance between the desired output vector y and the output vector ŷ that would now be produced by the neural network in response to receiving the input vector x. In many neural-network implementations, it is possible to make batched adjustments to the neural-network weights based on multiple output vectors produced from multiple inputs, as discussed further below.

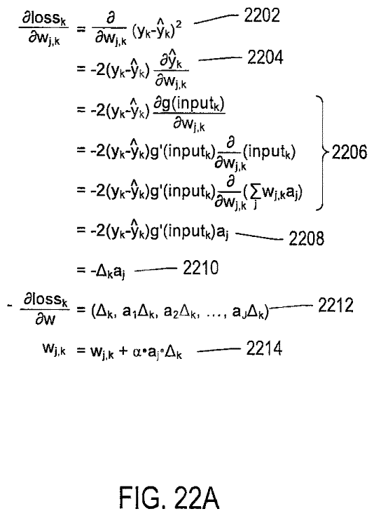

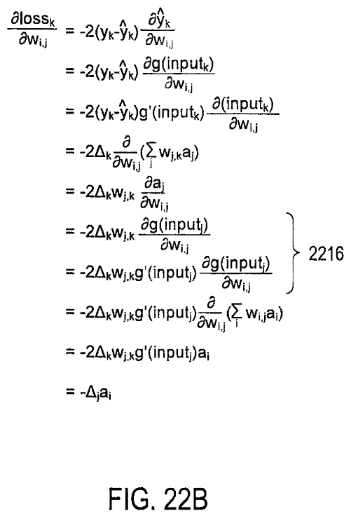

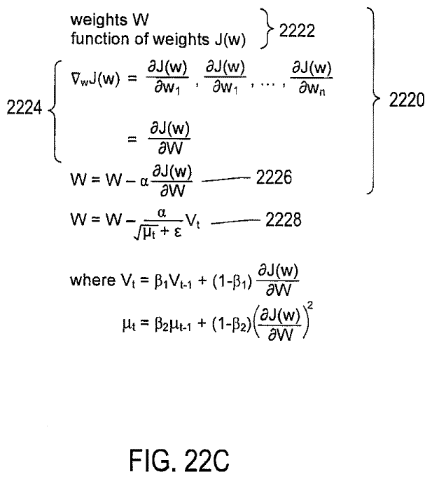

FIGS. 22A-C show details of the computation of weight adjustments made by neural-network nodes during backpropagation of error vectors into neural networks. The expression 2202 in FIG. 22A represents the partial differential of the loss, or k^{th} component of the squared length of the error vector eₖ², computed by the k^{th} output-layer neural-network node with respect to the J+1 weights applied to the formal 0^{th} input and inputs a₁-a_{J} received from higher-level nodes. Application of the chain rule for partial differentiation produces expression 2204. Substitution of the activation function for ŷₖ in the second application of the chain rule produces expressions 2206. The partial differential of the sum of weighted activations with respect to the weight for activation j is simply activation j, aⱼ, generating expression 2208. The initial factors in expression 2208 are replaced by −Δₖ to produce a final expression for the partial differential of the k^{th} component of the loss with respect to the j^{th} weight, 2210. The negative gradient of the weight adjustments is used in backpropagation in order to minimize the loss, as indicated by expression 2212. Thus, the j^{th} weight for the k^{th} output-layer neural-network node is adjusted according to expression 2214, where α is a learning-rate constant in the range [0, 1].

FIG. 22B illustrates computation of the weight adjustment for the kth component of the error vector in a final-hidden-layer neural-network node. This computation is similar to that discussed above with reference to FIG. 22A, but includes an additional application of the chain rule for partial differentiation in expressions 2216 in order to obtain an expression for the partial differential with respect to a second-hidden-layer-node weight that includes an output-layer-node weight adjustment.

FIG. 22C illustrates one commonly used improvement over the above-described weight-adjustment computations. The above-described weight-adjustment computations are summarized in expressions 2220, There is a set of weights W and a function of the weights J(W), as indicated by expressions 2222. The backpropagation of errors through the neural network is based on the gradient, with respect to the weights, of the function J(W), as indicated by expressions 2224. The weight adjustment is represented by expression 2226, in which a learning constant times the gradient of the function J(W) is subtracted from the weights to generate the new, adjusted weights. In the improvement illustrated in FIG. 22C, expression 2226 is modified to produce expression 2228 for the weight adjustment. In the improved weight adjustment, the learning constant α is divided by the sum of a weighted average of adjustments and a very small additional term ε and the gradient is replaced by the factor Vₜ, where t represents time or, equivalently, the current weight adjustment in a series of weight adjustments. The factor Vₜ is a combination of the factor for the preceding time point or weight adjustment Vₜ₋₁ and the gradient computed for the current time point or weight adjustment. This factor is intended to add momentum to the gradient descent in order to avoid premature completion of the gradient-descent process at a local minimum. Division of the learning constant α by the weighted average of adjustments adjusts the learning rate over the course of the gradient descent so that the gradient descent converges in a reasonable period of time.

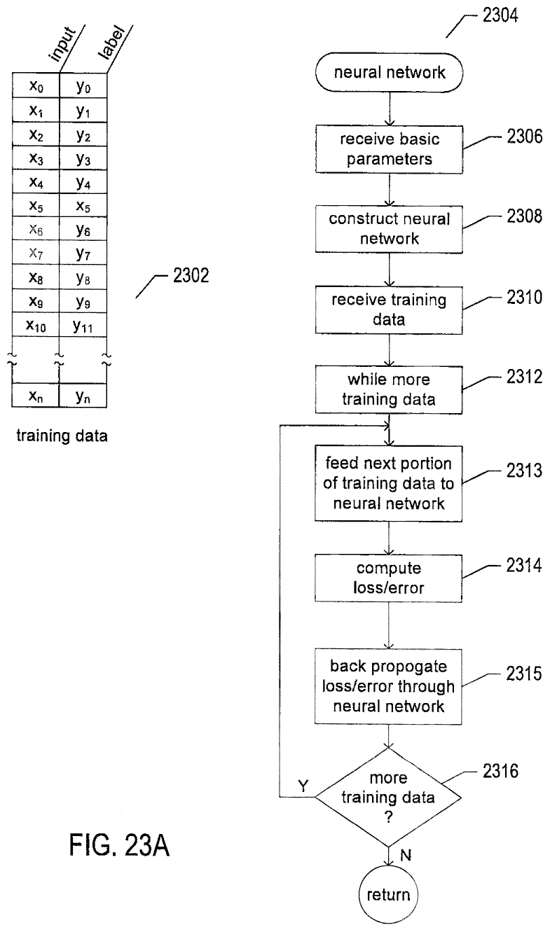

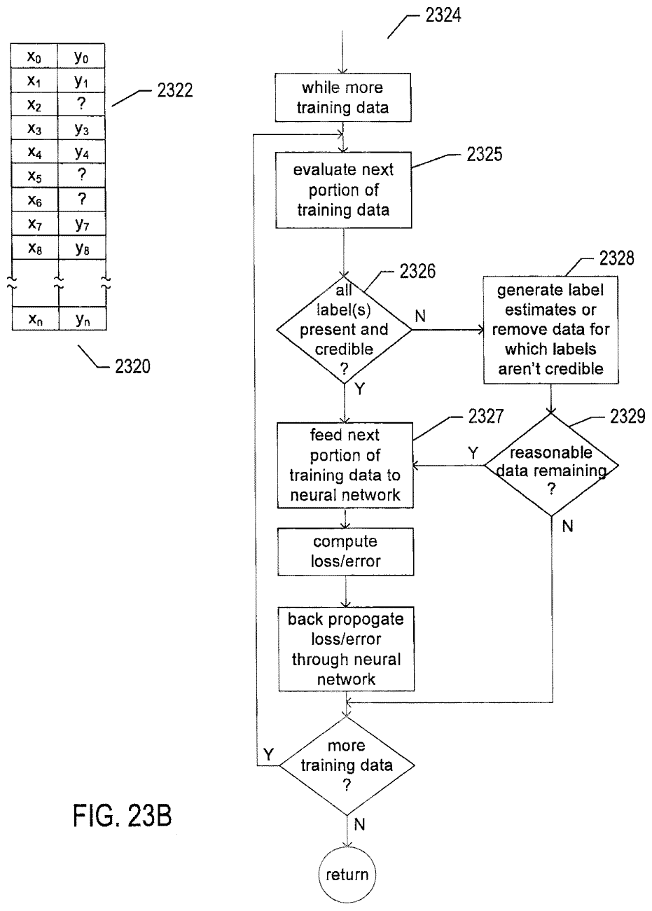

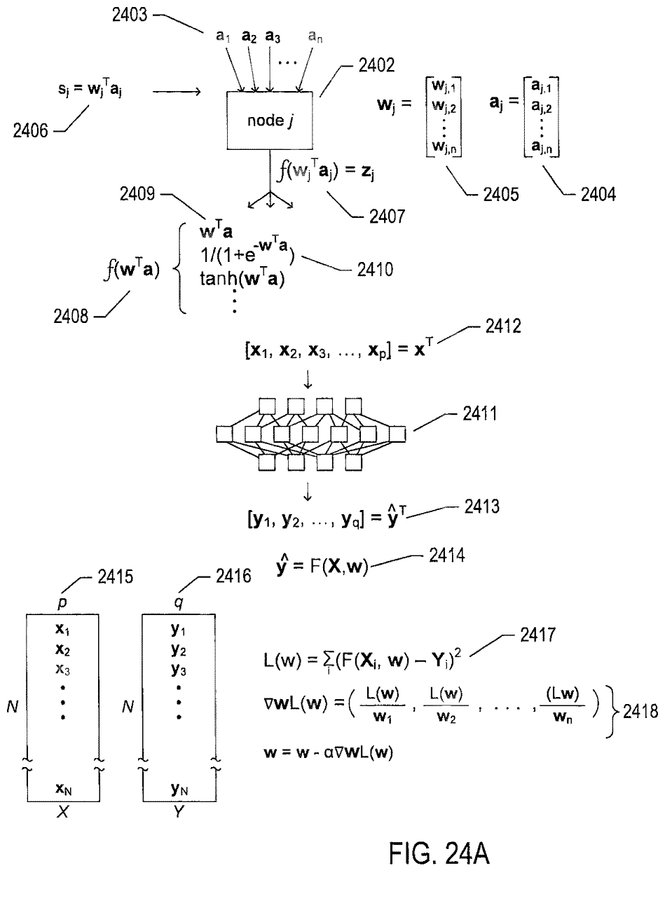

FIGS. 23A-B illustrate neural-network training. FIG. 23A illustrates the construction and training of a neural network using a complete and accurate training dataset. The training dataset is shown as a table of input-vector/label pairs 2302, in which each row represents an input-vector/label pair. The control-flow diagram 2304 illustrates construction and training of a neural network using the training dataset. In step 2306, basic parameters for the neural network are received, such as the number of layers, number of nodes in each layer, node interconnections, and activation functions. In step 2308, the specified neural network is constructed. This involves building representations of the nodes, node connections, activation functions, and other components of the neural network in one or more electronic memories and may involve, in certain cases, various types of code generation, resource allocation and scheduling, and other operations to produce a fully configured neural network that can receive input data and generate corresponding outputs. In many cases, for example, the neural network may be distributed among multiple computer systems and may employ dedicated communications and shared memory for propagation of activations and total error or loss between nodes. It should again be emphasized that a neural network is a physical system comprising one or more computer systems, communications subsystems, and often multiple instances of computer-instruction-implemented control components.

In step 2310, training data represented by table 2302 is received. Then, in the while-loop of steps 2312-2316, portions of the training data are iteratively input to the neural network, in step 2313, the loss or error is computed, in step 2311, and the computed loss or error is back-propagated through the neural network step 2315 to adjust the weights. The control-flow diagram refers to portions of the training data rather than individual input-vector/label pairs because, in certain cases, groups of input-vector/label pairs are processed together to generate a cumulative error that is back-propagated through the neural network. A portion may, of course, include only a single input-vector/label pair.

FIG. 23B illustrates one method of training a neural network using an incomplete training dataset. Table 2320 represents the incomplete training dataset. For certain of the input-vector/label pairs, the label is represented by a “?” symbol, such as in the input-vector/label pair 2322. The “?” symbol indicates that the correct value for the label is unavailable. This type of incomplete data set may arise from a variety of different factors, including inaccurate labeling by human annotators, various types of data loss incurred during collection, storage, and processing of training datasets, and other such factors. The control-flow diagram 2324 illustrates alterations in the while-loop of steps 2312-2316 in FIG. 23A that might be employed to train the neural network using the incomplete training dataset. In step 2325, a next portion of the training dataset is evaluated to determine the status of the labels in the next portion of the training data. When all of the labels are present and credible, as determined in step 2326, the next portion of the training dataset is input to the neural network, in step 2327, as in FIG. 23A. However, when certain labels are missing or lack credibility, as determined in step 2326, the input-vector label pairs that include those labels are removed or altered to include better estimates of the label values, in step 2328. When there is reasonable training data remaining in the training-data portion following step 2328, as determined in step 2329, the remaining reasonable data is input to the neural network in step 2327. The remaining steps in the while-loop are equivalent to those in the control-flow diagram shown in FIG. 23A. Thus, in this approach, either suspect data is removed, or better labels are estimated, based on various criteria, for substitution for the suspect labels.

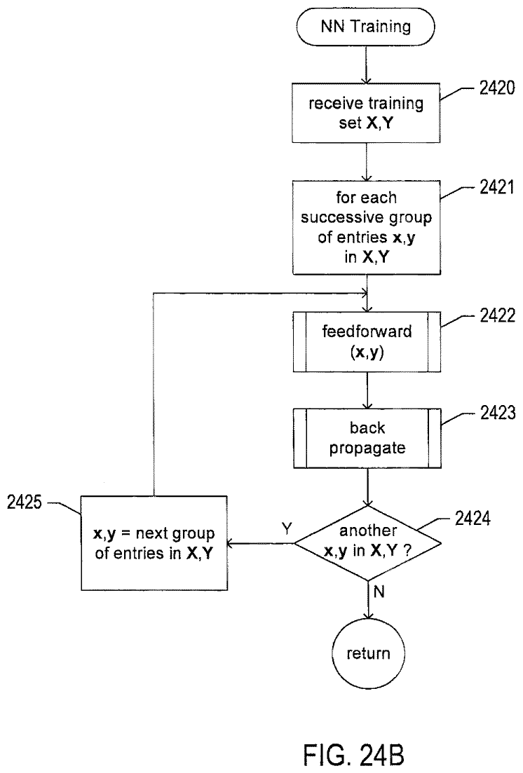

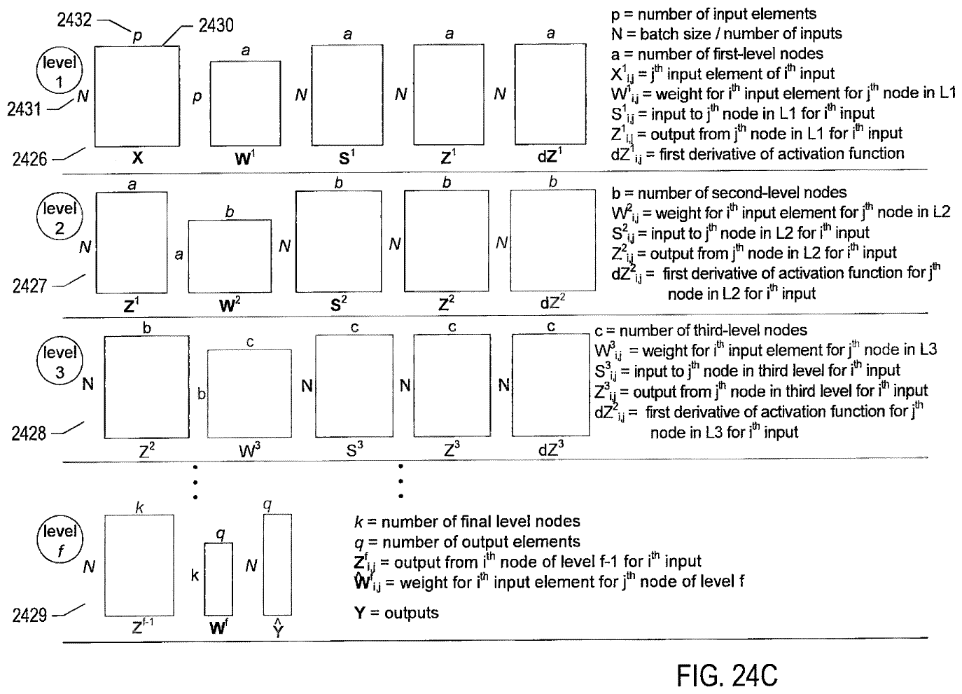

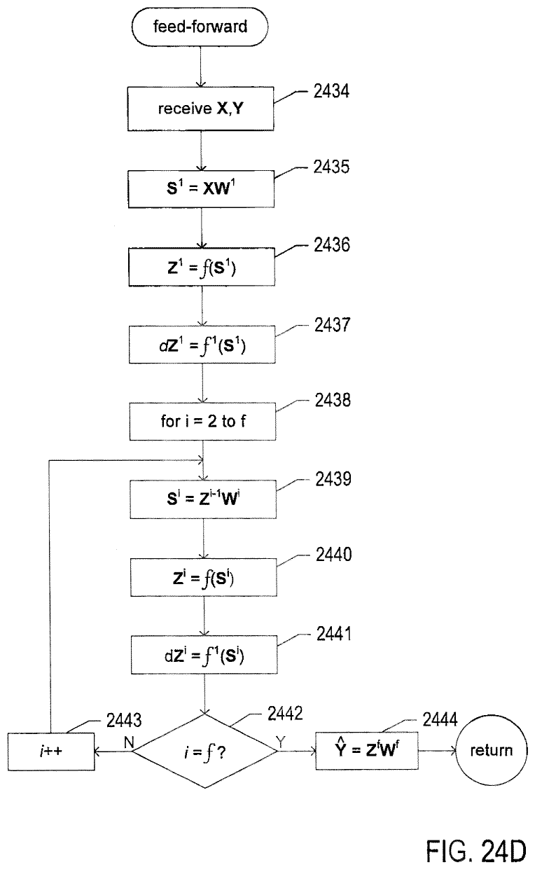

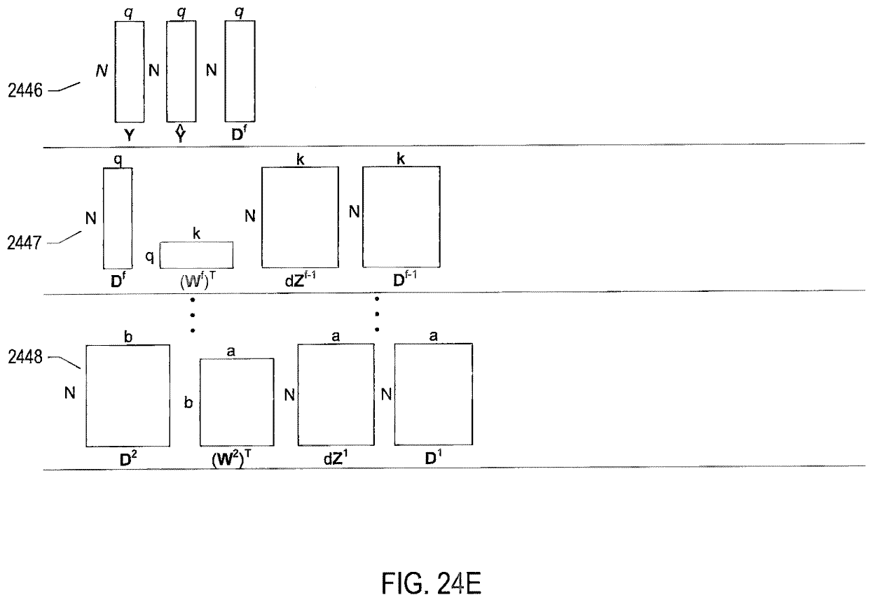

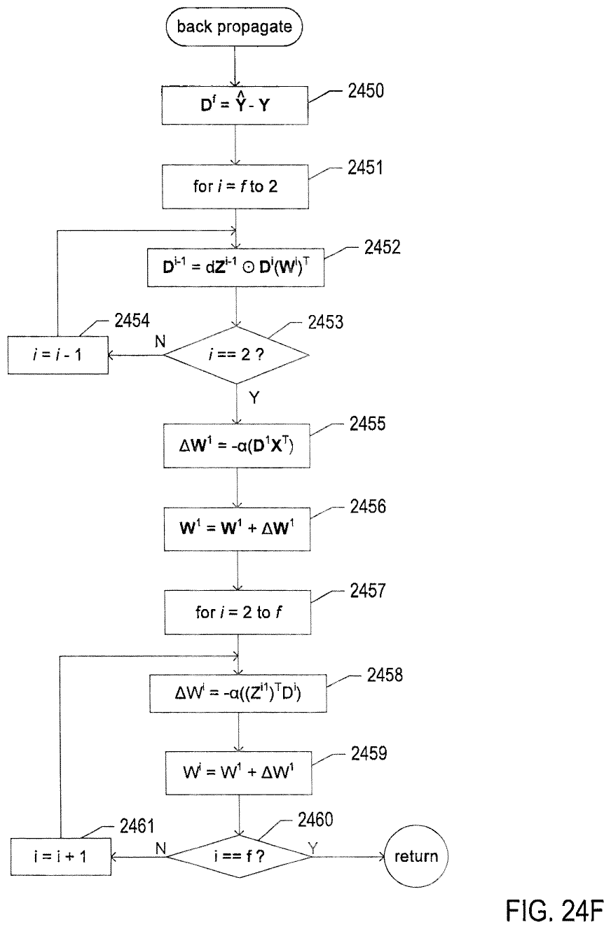

FIGS. 24A-F illustrate a matrix-operation-based batch method for neural-network training. This method processes batches of training data and losses to efficiently train a neural network. FIG. 24A illustrates the neural network and associated terminology. As discussed above, each node in the neural network, such as node J 2402, receives one or more inputs a 2403, expressed as a vector aⱼ 2404, that are multiplied by corresponding weights, expressed as a vector wⱼ 2405, and added together to produce an input signal sⱼ using a vector dot-product operation 2406. An activation function ƒ within the node receives the input signal s_{J} and generates an output signal z_{J} 2407 that is output to all child nodes of node j. Expression 2408 provides an example of various types of activation functions that may be used in the neural network. These include a linear activation function 2409 and a sigmoidal activation function 2410. As discussed above, the neural network 2411 receives a vector of p input values 2412 and outputs a vector of q output values 2413. In other words, the neural network can be thought of as a function F 2414 that receives a vector of input values x^{T} and uses a current set of weights w within the nodes of the neural network to produce a vector of output values ŷ^{T}. The neural network is trained using a training data set comprising a matrix X 2415 of input values, each of N rows in the matrix corresponding to an input vector X^{T}, and a matrix Y 2416 of desired output values, or labels, each of N rows in the matrix corresponding to a desired output-value vector y^{T}. A least-squares loss function is used in training 2417 with the weights updated using a gradient vector generated from the loss function, as indicated in expressions 2418, where a is a constant that corresponds to a learning rate.

FIG. 24B provides a control-flow diagram illustrating the method of neural-network training. In step 2420, the routine “NNTraining” receives the training set comprising matrices X and Y. Then, in the for-loop of steps 2421-2425, the routine “NNTraining” processes successive groups or batches of entries x and y selected from the training set. In step 2422, the routine “NNTraining” calls a routine “feedforward” to process the current batch of entries to generate outputs and, in step 2423, calls a routine “back propagated” to propagate errors back through the neural network in order to adjust the weights associated with each node.

FIG. 24C illustrates various matrices used in the routine “feedforward.” FIG. 24C is divided horizontally into four regions 2426-2429. Region 2426 approximately corresponds to the input level, regions 2427-2428 approximately correspond to hidden-node levels, and region 2429 approximately corresponds to the final output level. The various matrices are represented, in FIG. 24C, as rectangles, such as rectangle 2430 representing the input matrix X. The row and column dimensions of each matrix are indicated, such as the row dimension N 2431 and the column dimension p 2432 for input matrix X 2430. In the right-hand portion of each region in FIG. 24C, descriptions of the matrix-dimension values and matrix elements are provided. In short, the matrices W^{x} represent the weights associated with the nodes at level x, the matrices S^{x} represent the input signals associated with the nodes at level x, the matrices Z^{x} represent the outputs from the nodes at level x, and the matrices dZ^{x} represent the first derivative of the activation function for the nodes at level x evaluated for the input signals.

FIG. 24D provides a control-flow diagram for the routine “feedforward,” called in step 2422 of FIG. 24B. In step 2434, the routine “feedforward” receives a set of training data x and y selected from the training-data matrices X and Y. In step 2435, the routine “feedforward” computes the input signals S¹ for the first layer of nodes by matrix multiplication of matrices x and W¹, where matrix W¹ contains the weights associated with the first-layer nodes. In step 2436, the routine “feedforward” computes the output signals Z¹ for the first-layer nodes by applying a vector-based activation function ƒ to the input signals S¹. In step 2437, the routine “feedforward” computes the values of the derivatives of the activation function ƒ¹, dZ¹. Then, in the for-loop of steps 2438-2443, the routine “feedforward” computes the input signals S¹, the output signals Z, and the derivatives of the activation function dZ¹ for the nodes of the remaining levels of the neural network. Following completion of the for-loop of steps 2438-2443, the routine “feedforward” computes the output values ŷ^{T} for the received set of training data.

FIG. 24E illustrates various matrices used in the routine “back propagate.” FIG. 24E uses similar illustration conventions as used in FIG. 24C, and is also divided horizontally into horizontal regions 2446-2448. Region 2446 approximately corresponds to the output level, region 2447 approximately corresponds to hidden-node levels, and region 2448 approximately corresponds to the first node level. The only new type of matrix shown in FIG. 24E are the matrices D^{x} for node levels x. These matrices contain the error signals that are used to adjust the weights of the nodes.

FIG. 24F provides a control-flow diagram for the routine “back propagate.” In step 2450, the routine “back propagate” computes the first error-signal matrix D as the difference between the values ŷ output during a previous execution of the routine “feedforward” and the desired output values from the training set y. Then, in a for-loop of steps 2451-2454, the routine “back propagate” computes the remaining error-signal matrices for each of the node levels up to the first node level as the Shur product of the dZ matrix and the product of the transpose of the W matrix and the error-signal matrix for the next lower node level. In step 2455, the routine “back propagate” computes weight adjustments ΔW for the first-level nodes as the negative of the constant α times the product of the transpose of the input-value matrix and the error-signal matrix. In step 2456, the first-node-level weights are adjusted by adding the current W matrix and the weight-adjustments matrix ΔW. Then, in the for-loop of steps 2457-2461, the weights of the remaining node levels are similarly adjusted.

Thus, as shown in FIGS. 24A-F, neural-network training can be conducted as a series of simple matrix operations, including matrix multiplications, matrix transpose operations, matrix addition, and the Shur product. Interestingly, there are no matrix inversions or other complex matrix operations needed for neural-network training.

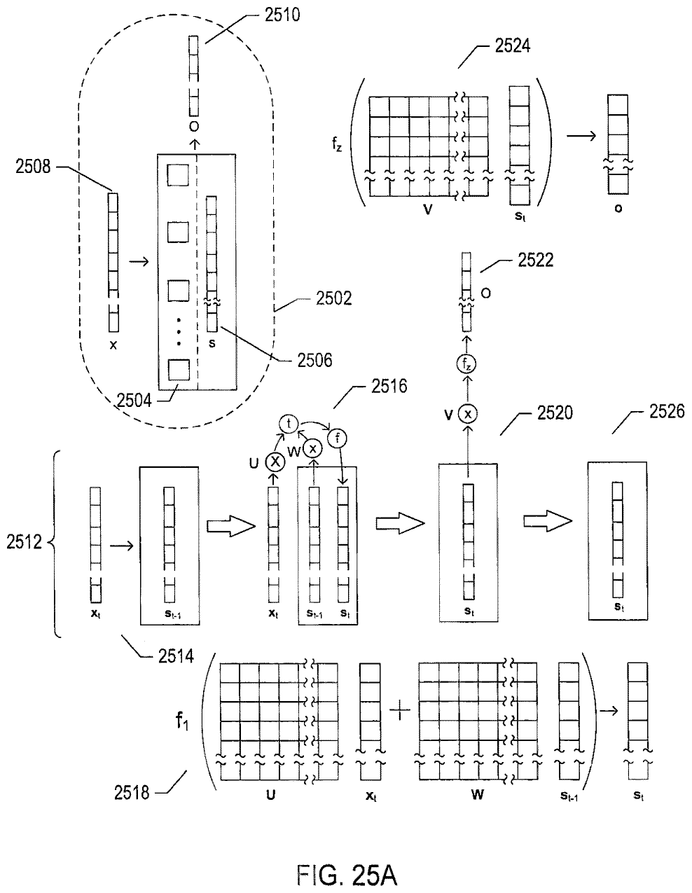

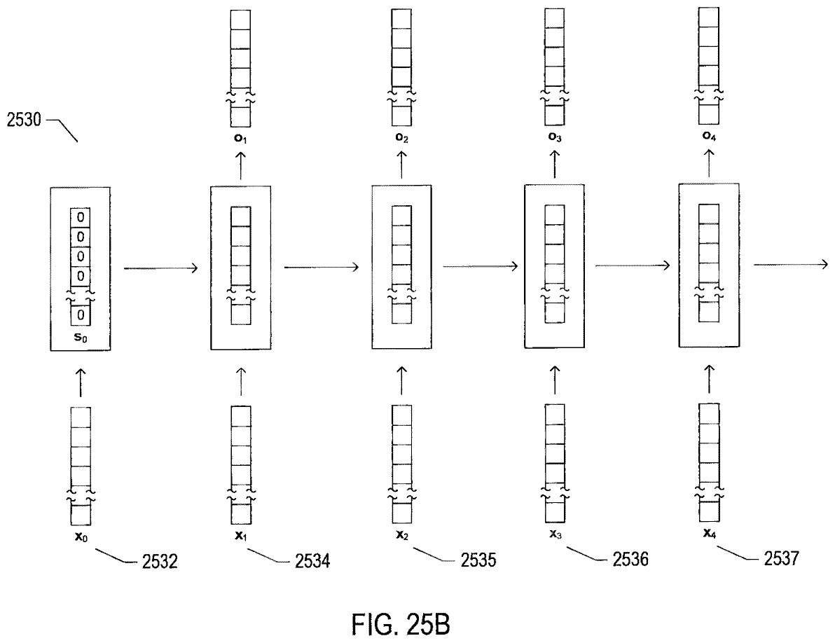

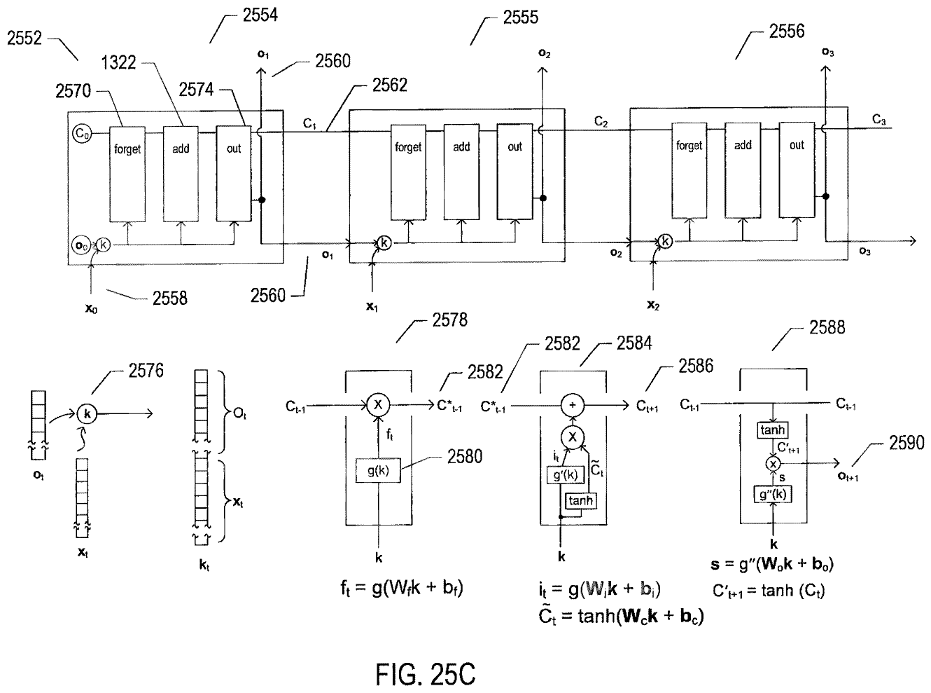

A second type of neural network, referred to as a “recurrent neural network,” is employed to generate sequences of output vectors from sequences of input vectors. These types of neural networks are often used for natural-language applications in which a sequence of words forming a sentence are sequentially processed to produce a translation of the sentence, as one example. FIGS. 25A-B illustrate various aspects of recurrent neural networks. Inset 2502 in FIG. 25A shows a representation of a set of nodes within a recurrent neural network. The set of nodes includes nodes that are implemented similarly to those discussed above with respect to the feed-forward neural network 2504, but additionally include an internal state 2506. In other words, the nodes of a recurrent neural network include a memory component. The set of recurrent-neural-network nodes, at a particular time point in a sequence of time points, receives an input vector x 2508 and produces an output vector 2510. The process of receiving an input vector and producing an output vector is shown in the horizontal set of recurrent-neural-network-nodes diagrams interleaved with large arrows 2512 in FIG. 25A. In a first step 2514, the input vector x at time t is input to the set of recurrent-neural-network nodes which include an internal state generated at time t−1. In a second step 2516, the input vector is multiplied by a set of weights U and the current state vector is multiplied by a set of weights W to produce two vector products which are added together to generate the state vector for time t. This operation is illustrated as a vector function ƒ₁ 2518 in the lower portion of FIG. 25A. In a next step 2520, the current state vector is multiplied by a set of weights V to produce the output vector for time t 2522, a process illustrated as a vector function ƒ₂ 2524 in FIG. 25A. Finally, the recurrent-neural-network nodes are ready for input of a next input vector at time t+1, in step 2526.

FIG. 25B illustrates processing by the set of recurrent-neural-network nodes of a series of input vectors to produce a series of output vectors. At a first time t₀ 2530, a first input vector x₀ 2532 is input to the set of recurrent-neural-network nodes. At each successive time point 2534-2537, a next input vector is input to the set of recurrent-neural-network nodes and an output vector is generated by the set of recurrent-neural-network nodes. In many cases, only a subset of the output vectors are used. Back propagation of the error or loss during training of a recurrent neural network is similar to back propagation for a feed-forward neural network, except that the total error or loss needs to be back-propagated through time in addition to through the nodes of the recurrent neural network. This can be accomplished by unrolling the recurrent neural network to generate a sequence of component neural networks and by then back-propagating the error or loss through this sequence of component neural networks from the most recent time to the most distant time period.

Finally, for completeness, FIG. 25C illustrates a type of recurrent-neural-network node referred to as a long-short-term-memory (“LSTM”) node. In FIG. 25C, a LSTM node 2552 is shown at three successive points in time 2554-2556. State vectors and output vectors appear to be passed between different nodes, but these horizontal connections instead illustrate the fact that the output vector and state vector are stored within the LSTM node at one point in time for use at the next point in time. At each time point, the LSTM node receives an input vector 2558 and outputs an output vector 2560. In addition, the LSTM node outputs a current state 2562 forward in time. The LSTM node includes a forget module 2570, an add module 2572, and an out module 2574. Operations of these modules are shown in the lower portion of FIG. 25C. First, the output vector produced a: the previous time point and the input vector received at a current time point are concatenated to produce a vector k 2576. The forget module 2578 computes a set of multipliers 2580 that are used to element-by-element multiply the state from time t−1 in order to produce an altered state 582. This allows the forget module to delete or diminish certain elements of the state vector. The add module 2134 employs an activation function to generate a new state 2586 from the altered state 2582. Finally, the out module 2588 applies an activation function to generate an output vector 2140 based on the new state and the vector k. An LSTM node, unlike the recurrent-neural-network node illustrated in FIG. 25A, can selectively alter the internal state to reinforce certain components of the state and deemphasize or forget other components of the state in a manner reminiscent of human short-term memory. As one example, when processing a paragraph of text, the LSTM node may reinforce certain components of the state vector in response to receiving new input related to previous input but may diminish components of the state vector when the new input is unrelated to the previous input, which allows the LSTM to adjust its context to emphasize inputs close in time and to slowly diminish the effects of inputs that are not reinforced by subsequent inputs. Here again, back propagation of a total error or loss is employed to adjust the various weights used by the LSTM, but the back propagation is significantly more complicated than that for the simpler recurrent neural-network nodes discussed with reference to FIG. 25A.

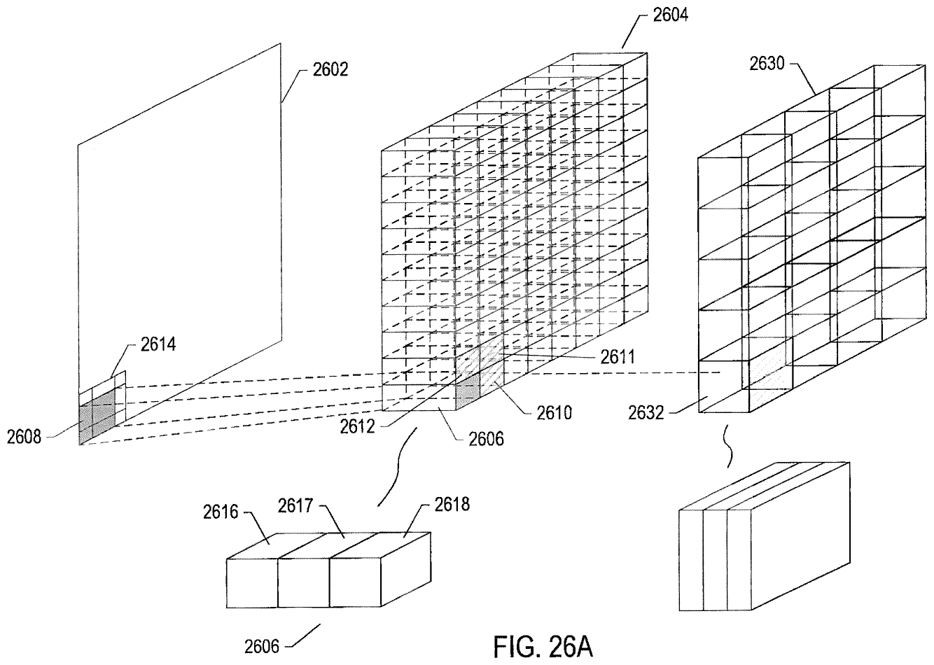

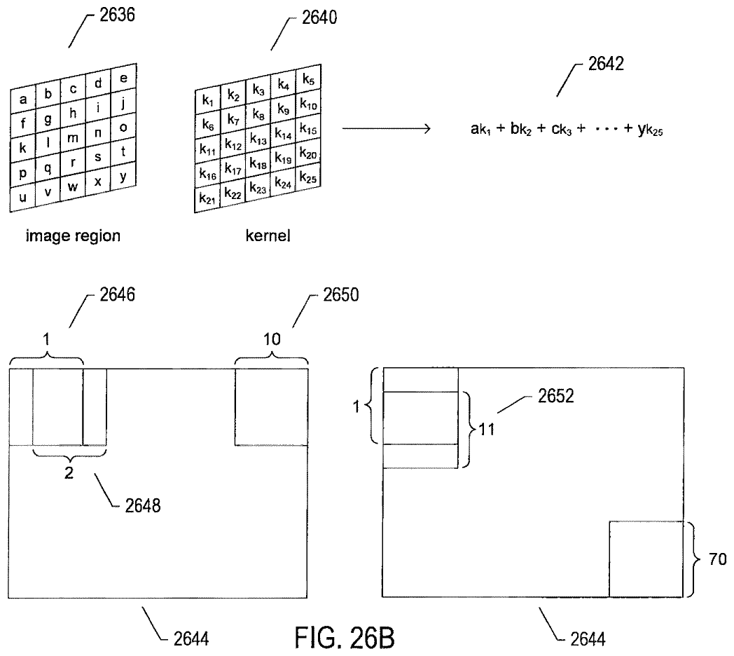

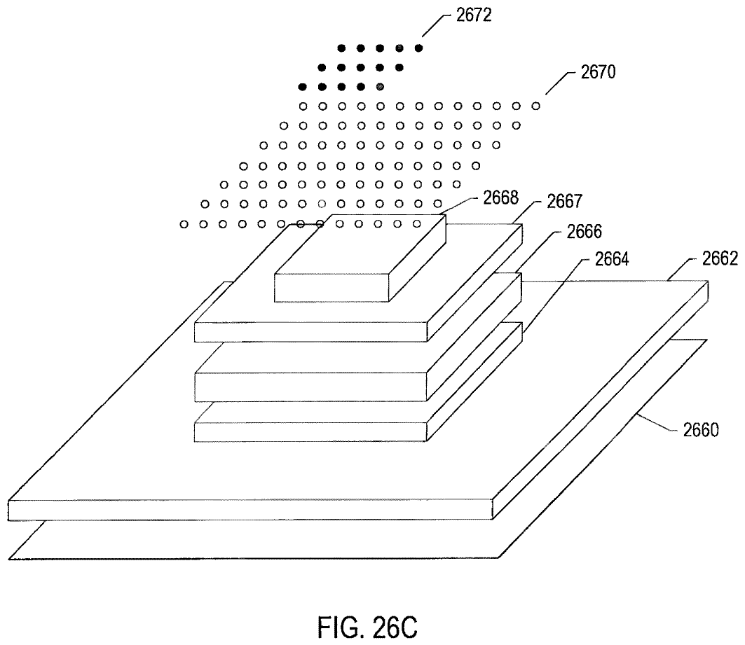

FIGS. 26A-C illustrate a convolutional neural network. Convolutional neural networks are currently used for image processing, voice recognition, and many other types of machine-learning tasks for which traditional neural networks are impractical. In FIG. 26A, a digitally encoded screen-capture image 2602 represents the input data for a convolutional neural network. A first level of convolutional-neural-network nodes 2604 each process a small subregion of the image. The subregions processed by adjacent nodes overlap. For example, the corner node 2606 processes the shaded subregion 2608 of the input image. The set of four nodes 2606 and 2610-2612 together process a larger subregion 2614 of the input image. Each node may include multiple subnodes. For example, as shown in FIG. 26A, node 2606 includes 3 subnodes 2616-2618. The subnodes within a node all process the same region of the input image, but each subnode may differently process that region to produce different output values. Each type of subnode in each node in the initial layer of nodes 2604 uses a common kernel or filter for subregion processing, as discussed further below. The values in the kernel or filter are the parameters, or weights, that are adjusted during training. However, since all the nodes in the initial layer use the same three subnode kernels or filters, the initial node layer is associated with only a comparatively small number of adjustable parameters. Furthermore, the processing associated with each kernel or filter is more or less translationally invariant, so that a particular feature recognized by a particular type of subnode kernel is recognized anywhere within the input image that the feature occurs. This type of organization mimics the organization of biological image-processing systems. A second layer of nodes 2630 may operate as aggregators, each producing an output value that represents the output of some function of the corresponding output values of multiple nodes in the first node layer 2604. For example, second-a layer node 2632 receives, as input, the output from four first-layer nodes 2606 and 2610-2612 and produces an aggregate output. As with the first-level nodes, the second-level nodes also contain subnodes, with each second-level subnode producing an aggregate output value from outputs of multiple corresponding first-level subnodes.

FIG. 26B illustrates the kernel-based or filter-based processing carried out by a convolutional neural network node. A small subregion of the input image 2636 is shown aligned with a kernel or filter 2640 of a subnode of a first-layer node that processes the image subregion. Each pixel or cell in the image subregion 2636 is associated with a pixel value. Each corresponding cell in the kernel is associated with a kernel value, or weight. The processing operation essentially amounts to computation of a det product 2642 of the image subregion and the kernel, when both are viewed as vectors. As discussed with reference to FIG. 26A, the nodes of the first level process different, overlapping subregions of the input image, with these overlapping subregions essentially tiling the input image. For example, given an input image represented by rectangles 2644, a first node processes a first subregion 2646, a second node may process the overlapping, right-shifted subregion 2648, and successive nodes may process successively right-shifted subregions in the image up through a tenth subregion 2650. Then, a next down-shifted set of subregions, beginning with an eleventh subregion 2652, may be processed by a next row of nodes.

FIG. 26C illustrates the many possible layers within the convolutional neural network. The convolutional neural network may include an initial set of input nodes 2660, a first convolutional node layer 2662, such as the first layer of nodes 2604 shown in FIG. 26A, and aggregation layer 2664, in which each node processes the outputs for multiple nodes in the convolutional node layer 2662, and additional types of layers 2666-2668 that include additional convolutional, aggregation, and other types of layers. Eventually, the subnodes in a final intermediate layer 2668 are expanded into a node layer 2670 that forms the basis of a traditional, fully connected neural-network portion with multiple node levels of decreasing size that terminate with an output-node level 2672.

### Neural Networks Used for Mental-State-Attribute-Value Detection in the Currently Disclosed Mental-State-Monitoring and Stimulus-Generation System

This section provides details regarding processing EEG monitoring data and using the processed EEG monitoring data to detect mental-state attributes. EEG data are typically collected using electrodes placed on the scalp to measure the electrical activity of the brain. The data are recorded as a continuous time series, with each sample representing the electrical activity at a specific point in time. Assuming in_chans channels (electrodes) and T time steps, the raw EEG data can be represented as a 2-dimensional matrix X, where each row corresponds to a channel and each column corresponds to a time step:

$$X ∈ {R}^{in\_chans \times T} .$$

The initial processing steps, carried out by the signal-demultiplexer component (1404 in FIG. 14) of an SP/SD component of the DMS application, are next described. The initial processing steps include: (1) filtering; (2) epoching; (3) cropping; (4) artifact rejection; and (5) normalization.

The first filtering preprocessing step is used, in EEG data analysis, to remove unwanted noise and artifacts and to isolate the neural signals of interest. EEG signals are often contaminated with various types of noise, including electrical interference from the environment or the recording equipment, physiological noise from the body's own biological activity, and movement artifacts from the subject's body movements. Filtering is used to remove these unwanted frequencies from the EEG data and enhance the signal-to-noise ratio. A common approach is to use a bandpass filter, which removes frequencies outside a specific range of interest. For example, it is often beneficial to filter out ambient alternating current (“AC”) frequencies, which can contaminate the EEG signal with electrical interference from the surrounding environment.

EEG data are often collected in short segments known as epochs, each lasting, say, a few seconds. The second epoching preprocessing step allows the data to be analyzed in discrete time intervals and can help identify specific brain states or events. This can be represented mathematically as:

$$Y = epoch ( X , epoch\_length , S )$$

where Y is the epoched EEG data, epoch_length is the epoch length in time steps, and S is the epoch stride (i.e., the number of time steps between successive epochs). Given an input EEG signal X of shape (in_chans, T), where in_chans is the number of channels and T is the number of time steps, the epoching process is represented as follows:

$${Y}_{i , j} = {X}_{j , S \cdot i : S \cdot i + epo} \qquad {}_{\_ leng}$$

where Y is the epoched EEG data of shape (num_epochs, in_chans, epoch_length); epoch_length is the epoch length in time steps; S is the epoch stride (i.e., the number of time steps between successive epochs), i∈(0, 1, . . . num_epochs−1) is the index of the epoch, j∈{0, 1, . . . , in_chans−1} is the index of the channel, and

$$num\_epochs = ⌊ \frac{\begin{matrix}T - epo & \_leng\end{matrix}}{S} ⌋ + 1.$$

The expression for Y_{i,j} indicates that the i^{th} epoch of the j^{th} channel, Y_{i,j}, is obtained by taking a slice of the input EEG signal Xⱼ with a length of epoch_length time steps, starting from the position s·i.

The third preprocessing step, cropping, extracts one or more smaller segments of length crop_size from each epoch. This results in a cropped EEG data tensor, Y_{c}∈R_{num_crops×in_chans×crop_size}, where

$$num\_crops = num\_epochs \times num\_crops \_per \_epoch .$$

Cropping is a form of data augmentation that increases the effective size of the dataset by creating multiple smaller segments (crops) from each epoch. This can lead to better generalization and improved model performance, as the model is exposed to more varied samples during training. As cropping generates multiple crops from each epoch, it introduces variability into the training data. This variability acts as an implicit regularization technique, helping to prevent overfitting and allowing the model to better generalize to new, unseen data. In some applications, the specific timing of neural events within an epoch may not be crucial to the task at hand. Cropping allows the model to learn features that are invariant to the exact timing of these events, making the model more robust to variations in the data. Training deep learning models on smaller crops of the data can be computationally more efficient. Smaller input sizes require less memory and computational resources, allowing for faster training and enabling the use of larger models or more complex architectures. By training the model on smaller crops, the optimization process can be more focused on learning the most relevant features within each crop. This can result in more stable training dynamics and potentially faster convergence.

The fourth preprocessing step is artifact rejection, EEG data can contain various types of artifacts, such as those caused by eye blinks or other types of body movements. These artifacts can be identified and removed using various techniques, such as independent component analysis (ICA) or template matching.

The fifth preprocessing step, normalization, is used to standardize the EEG data across channels and time steps. A common approach is to process the epochs in each channel by subtracting the mean and dividing by the standard deviation of the data across the epochs in the channel. This can be represented mathematically as:

$$Y = \frac{X - μ}{σ}$$

where Y is the normalized EEG data for a channel. X is the raw EEG data for the channel, and μ and σ are the mean and standard deviation of the data in the channel, respectively.

Once these preprocessing steps have been applied, the epoched and cropped EEG data are organized as a 3-dimensional tensor. Y_{c}∈R^{num_crops×in_chans×crop_size}. The parameter input_window_samples is set as follows:

$$input\_window \_samples ≡ crop\_size .$$

Machine learning methods make heavy use of batches. Batches are small subsets of the training data that are used to train machine learning procedures, especially neural networks, in an iterative manner. Instead of processing the entire dataset at once, the dataset is divided into smaller chunks called batches. Each batch is then fed into the method during training, and the model updates its weights based on the error it made on that specific batch. This process is repeated until all batches have been processed, which constitutes one complete iteration over the dataset, also known as an epoch. The training process typically consists of multiple epochs. There are several reasons why machine learning procedures, particularly deep learning models, work with batches. Processing large datasets all at once can be computationally expensive and might not fit in the memory of the computing device (e.g., GPU). Working with smaller batches allows the model to be trained on more modest hardware resources. When training deep learning models, a popular optimization technique called stochastic gradient descent (SGD) is used. SGD introduces an element of randomness in the optimization process by updating the model's weights using a random subset of the data (a batch) instead of the entire dataset. This randomness helps the model escape local minima and find better solutions. Training with batches can act as a form of implicit regularization, which helps prevent overfitting. The noise introduced by using a subset of the data in each iteration can make the model more robust and generalize better to unseen data. Since weight updates are made more frequently when using batches, the model often converges faster than when using the full dataset in each iteration. This can lead to a shorter overall training time. In summary, training with batches provides computational benefits, helps with optimization, and can improve the model's generalization capabilities.

To form an input tensor X∈R^{batch_size×in_chans×input_window_samples} for a machine learning procedure, non-overlapping groups of cropped epochs are sampled from Y_{c} and stacked along the first dimension to create a batch. This process can be represented as:

$${X}_{n , : , :} = {Y}_{c , batch\_index ( n ) , : , :} , \qquad for n = 0 , … , batch\_size - 1$$

where batch_index(n) is a function that maps the index n in the batch to the corresponding epoch index in Y_{c}. Note that the total number of batches that can be created from Y_{c} is given by ┌num_epochs/batch_size┐. During training, these batches of data are iteratively fed to the neural network. To form an input tensor X∈R^{batch_size×in.chans×input_window_samples} for the machine learning method, non-overlapping groups of cropped epochs from Y_{c} are sampled and stacked along the first dimension to create a batch. This process can be represented as:

$${X}_{n , : , :} = {Y}_{c , batch\_index ( n ) , : , :} , \qquad for n = 0 , … , batch\_size - 1$$

where batch_index(n) is a function that maps the index n in the batch to the corresponding epoch index in Y_{c}. Note that the total number of batches that can be created from Y_{c} is given by ┌num_crops/batch_size┐. During training, these batches of data are fed iteratively to the neural network.

An architecture that can be used to train and deploy a method to classify an EEG signal, assigning probabilities that the signal is associated with each of n_classes categories, is next described. There are many variations of the model implementation as described below, as well as some possible variations and enhancements. For example, this method to build a model is used when it is desired to estimate, given the subject's EEG signal data over an observation period, the probabilities that a subject will or will not (k=2) experience an epileptic seizure over a given subsequent time horizon. This method is trained using a dataset consisting of many EEG recordings for many subjects, along with labels (for the epilepsy example, 0 or 1, according to whether the subject experienced an epileptic seizure over a given subsequent time horizon).

Given EEG input X∈R^{batch_size×in_chans×input_window_samples}, the architecture of the method consists of the following pieces or layers which are applied sequentially: (1) expand tensor to 4 dimensions: (2) shuffle dimensions; (3) first convolutional block; (4) later convolutional blocks: (5) convolutional classifier; (6) softmax; and (7) squeeze.

A deep convolutional neural network (“CNN”) that makes use of 2D convolution layers is described below. CNNs, originally designed for image processing, typically use 4-dimensional input tensors. In order to make use of standard software and libraries, the tensor shape is expanded to 4 dimensions by adding a singleton final dimension, insuring that the input tensor has shape R^{batch,size×in_cha×input_window_samples×1}.

The dimensions of the input tensor are reordered, placing the spatial channels at the end and inserting a singleton dimension as the second dimension. That is, given the input tensor with dimensions: (batch_size, in_chans, input_window_samples, 1). the dimensions are shuffled so that the output tensor has dimensions: (batch_size, 1, input_window_samples, in_chans). Specifically, this is done to match the expected input format for 2D convolutional layers in currently available deep learning frameworks. In these frameworks, designed to handle images, the 2D convolutional layers expect input tensors to have the following format: (batch_size, num_feature_maps, height, width). Here, num_feature_maps represents the number of feature maps in the input tensor, and height and width represent the spatial dimensions. The parameter num_feature_maps in the context of images are explained as follows. Images are of two types: (1) Grayscale, num_feature_maps=1, with the value representing the intensity of the pixels on a scale from black to white; and (2) RGB (Red, Green, Blue), num_feature_maps=3, with each feature map representing the intensity of the red, green, and blue components for each pixel. EEG data can be viewed as analogous to a grayscale image, with a height corresponding to the number of time steps (input_window_samples) and a width corresponding to the number of electrodes (in_chans). It is therefore appropriate to shuffle dimensions so that the output sensor has dimensions:
(batch_size, 1, input_window_samples, in_chans).

The first block contains two convolutional layers-a temporal convolution and spatial convolution.

The temporal convolution layer captures the local temporal patterns in the input EEG data. It does this by applying a series of filters to the data, where each filter is designed to detect specific temporal features. By convolving these filters with the input data, the layer can extract useful information about the underlying brain activity and represent it in a more compact form. This layer helps the model to learn temporal relationships in the data, and is advantageous with respect to identifying specific events or cognitive states based on the temporal patterns in the EEG signals.

The convolution operator with bias can be expressed as:

$${Y}_{i , j , k , l} = {\sum }_{m = 0}^{filter\_time \_length - 1} {X}_{i , 0 , k + m , l} \cdot {W}_{j , 0 , m , 0} + {b}_{j}$$

where i represents the batch index; k represents the output time step index; l represents the output spatial index (electrode): X is the input tensor with dimensions (batch_size, 1, input_window_samples, in chans): W is the convolutional kernel with dimensions (n_filters_time, 1, filter_time_length, 1): b is the bias term with dimension (n_filters_time); and Y is the output tensor with dimensions (batch_size, n_filters_time, T_out, in_chans), with

$$T\_out = input\_window \_samples - filter\_time \_length + 1.$$

The summation iterates over the temporal dimension, filter_time_length, to perform the convolution operation. The ranges of the indices are as follows: i∈[0≤i≤batch_size−1]; j∈[0≤j≤n_filters_time−1]; [k∈[0≤k≤Tₒᵤₜ−1]; and l∈[0≤l≤in_chans−1]. The bias term bⱼ is added after the summation to obtain the final output value for each element in the output tensor Y.

The input to the spatial convolution layer is the output of the time convolution layer, which has shape (batch_size, n_filters_time, T_out, in_chans). The spatial convolution layer applies a 2D convolution with a kernel of shape (1, in_chans) and stride (conv_stride, 1). The kernel's spatial extent is equal to the number of input channels (in_chans), effectively applying a separate weight to each channel for each spatial filter. This operation is performed independently for each of the n_filters_time input feature maps. The spatial convolution operation can be expressed as:

$${Z}_{i , j , k , 0} = {\sum }_{m = 0}^{in\_chans} {Y}_{i , n , k , m} \cdot {W}_{j , n , 0 , m} + {b}_{j} .$$

where i represents the batch index: j represents the spatial filter index; k represents the output time step index; and n represents the input feature map index (from the time convolution layer); Y is the input tensor (output of the time convolution layer) with dimensions (batch_size, n_filters_time, T_out, in_chans); W is a convolutional kernel with dimensions (n_filters_spat, n_filters_time, 1, in_chans); b is a bias term with dimensions (n_filrers_spat); and Z is an output tensor with dimensions (batch_size, n_filters_spat, T_out, 1). The summation iterates over the input channels, effectively collapsing the channel dimension and resulting in an output tensor with shape (batch_size, n_filters_spat, T_out, 1). After the spatial convolution just described, the following are performed to complete processing in the first block: (1) layer normalization; (2) nonlinear convolution; (3) pooling; and (4) nonlinear pooling.

Batch normalization addresses the problem of internal covariate shift, which occurs when the distribution of the input data changes during training. By normalizing the input data, batch normalization helps to stabilize and speed up the training process. However, normalizing the data (i.e., forcing the activations to have zero mean and unit variance) might not always be the best representation for the model to learn. Some layers might perform better with different scales or shifts in the activations. So scaling and offset control variables, γ_{c} and β_{c}, are introduced. The scaling and offset control variables allow the model to learn the best scale and shift for the normalized activations. In other words, they enable the model to control the scale and shift of the activations while still benefiting from the stabilization provided by normalization. This provides the model with the flexibility to learn different feature scales and shifts that are best suited for the task at hand.

The input tensor, X, has dimensions: (batch_size, n_filters_spat, T_out, 1) and the output tensor, Y has the same dimensions: (batch_size, n_filters_spat, T_out, 1).

During training:

$${μ}_{c} = \frac{1}{batch\_size \cdot T\_out} {\sum }_{n = 1}^{batch\_size} {\sum }_{t = 1}^{T\_out} {X}_{n , c , t , 1} ,$$

$${σ}_{c}^{2} = \frac{1}{batch\_size \cdot T\_out} {\sum }_{n = 1}^{batch\_size} {\sum }_{t = 1}^{T\_out} {( {X}_{n , c , t , 1} - {μ}_{c} )}^{2} ,$$

$${\hat{X}}_{n , c , t , 1} = \frac{{X}_{n , c , t , 1} - {μ}_{c}}{\sqrt{{σ}_{c}^{2} + ϵ}} ,$$

$$and$$

$${Y}_{n , c , t , 1} = {γ}_{c} {\hat{X}}_{n , c , t , 1} + {β}_{c} .$$

During testing:

$${Y}_{n , c , t , 1} = {γ}_{c} \frac{{X}_{n , c , t , 1} - {μ}_{c}}{\sqrt{{σ}_{c}^{2} + ϵ}} + {β}_{c}$$

Running mean and variance are updated via:

$${running\_mean}_{c} = batch\_norm \_alpha \cdot {running\_mean}_{c} + ( 1 - batch\_norm \_alpha ) \cdot {μ}_{c} \\ and \\ {running\_var}_{c} = batch\_norm \_alpha \cdot {running\_var}_{c} + ( 1 - batch\_norm \_alpha ) \cdot {σ}_{c}^{2} ,$$

where the momentum for batch normalization is determined by the hyperparameter batch_norm_alpha.

The running mean and running variance are used to maintain an estimate of the mean and variance of the feature values during the training process. These running estimates are then used during the inference (testing, phase to normalize the input data. The reason for using the running mean and variance during inference is that the actual mean and variance of a single batch might not be representative of the entire dataset. During the training phase, for each mini-batch, the mean and variance are computed using the current batch data (as shown in the equations for μ_{c} and σ_{c}²). Then, the running mean and running variance are updated using an exponential moving average (EMA), which considers both the current batch mean and variance as well as the previous running mean and variance. The EMA update is controlled by the hyperparameter batchₙormₐlpha, which determines the momentum for batch normalization. During the inference (testing, phase, the running mean and running variance, which are the estimates of the mean and variance of the entire dataset, are used to normalize the input data (as shown in the equation for Y_{n,c,t,1} during testing). This allows for more consistent and accurate predictions, as the normalization statistics are representative of the entire dataset, rather than a single batch.

Like batch normalization, layer normalization stabilizes and speeds up the training process by normalizing the activations. However, instead of normalizing across the batch dimension, layer normalization normalizes across the feature dimension. This is particularly advantageous when the batch size is small or when working with recurrent neural networks, as it allows for stable training without being sensitive to the batch size. Similar to batch normalization, layer normalization also introduces scaling and offset parameters, γ_{c} and β_{c}, allowing the model to learn the best scale and shift for the normalized activations. The input tensor, X, has dimensions: (batch_size, n_filters_spat, T_out, 1) and the output tensor, Y has the same dimensions: (batch_size, n_filters_spat, T_out, 1). For both training and testing:

$${μ}_{n} = \frac{1}{n\_filters \_spat \cdot T\_out} {\sum }_{c = 1}^{n \_ filters \_ spat} {\sum }_{t = 1}^{T \_ out} {X}_{n , c , t , 1} , \\ {σ}_{n}^{2} = \frac{1}{n\_filters \_spat \cdot T\_out} {\sum }_{c = 1}^{n \_ filters \_ spat} {\sum }_{t = 1}^{T \_ out} {( {X}_{n , c , t , 1} - {μ}_{n} )}^{2} , \\ {\hat{X}}_{n , c , t , 1} = \frac{{X}_{n , c , t , 1} - {μ}_{n}}{\sqrt{{σ}_{c}^{2} + ϵ}} , \\ and \\ {Y}_{n , c , t , 1} = {γ}_{c} {\hat{X}}_{n , c , t , 1} + {β}_{c} .$$

In layer normalization, the mean and variance are computed for each instance in the batch independently. The mean and variance are calculated along the feature dimensions n_filters_spat and T_out. The normalization step is the same for both the training and testing phases. The layer normalization is independent from the batch size. This property makes it particularly advantageous in situations where the batch size is small or variable, such as in recurrent neural networks or when working with small datasets.

The Exponential Linear Unit (ELU) activation function is provided below:

$${f}_{ELU} ( x ) = \{ \begin{matrix}x , & if x > 0 \\ α ( {e}^{x} - 1 ) , & if x \leq 0\end{matrix}$$

The Exponential Linear Unit (ELU), activation function introduces nonlinearity into the deep learning model. Convolutional layers and other linear operations in the model cannot capture complex, nonlinear relationships present in the input data. By applying a nonlinear activation function like ELU, the model can learn these complex patterns and generalize better to new data. The ELU activation function over other activation functions has the following advantageous properties: (1) smoothness; (2) non-zero gradients for negative inputs; (3) faster learning; (4) saturation; (5) saturation.

ELU is a smooth function, which means that its derivative is continuous for all input values. This smoothness can help improve the stability of gradient-based optimization procedures, such as stochastic gradient descent, as there are no abrupt changes in the gradient. Unlike the Rectified Linear Unit (ReLU) activation function, which has a zero gradient for negative input values. ELU has non-zero gradients for negative inputs. This property helps to mitigate the “dying ReLU” problem, where ReLU neurons can become inactive and stop learning during training if their gradients are consistently zero. The negative values for the activation function in the ELU help push the mean activations closer to zero. This can lead to faster learning and improved generalization compared to activation functions like ReLU, which have only non-negative activation values. ELU has a saturation region for negative input values, which can help reduce the effect of exploding gradients in very deep networks. ELU has a saturation region for negative input values, which can help reduce the effect of exploding gradients in very deep networks. Overall, the ELU activation function can lead to more robust and efficient training of deep learning models, especially when dealing with complex and highly nonlinear data.

Pooling reduces the spatial dimensionality of the input tensor. Pooling is an operation used to reduce the spatial dimensionality of the input tensor by aggregating neighboring values, where pool_time_length defines the size of the pooling window and pool_time_stride determines the step size between successive pooling windows. Two common types of pooling are max pooling and mean pooling. The mux pooling operation is used in the described implementation. Max pooling can be expressed as follow:

$${Y}_{n , c , t ′ , 1} = {max}_{t ∈ P ( t ′ )} {X}_{n , c , t , 1}$$

Means pooling can be expressed as:

$${Y}_{n , c , t ′ , 1} = \frac{1}{pool time length} {\sum }_{t ∈ P ( t ′ )} {X}_{n , c , t , 1} \\ where P ( {t}^{′} ) = \{ {t}^{′} \cdot pool\_time \_stride . {t}^{′} \cdot pool\_time \_stride + 1 , \\ … {t}^{′} \cdot pool\_time \_stride + pool\_time \_length - 1 \} . \\ n ∈ \{ 0 , 1 , … , batch\_size - 1 \} \\ c ∈ \{ 0 , 1 , … , n\_filters \_spat - 1 \} \\ {t}^{′} ∈ \{ 0 , 1 , … , {T}^{′} - 1 \}$$

and the output temporal dimension. T′, is determined by the pooling operation and is given by

$${T}^{′} = ⌊ \frac{T\_out - pool\_time \_length}{pool\_time \_stride} + 1 ⌋ .$$

The input tensor dimension are: (batch_size, n_filters_spat, T_out, 1) and the output tensor dimensions are: (batch_size, n_filters_spat, T′, 1).

An activation function follows pooling. In general this layer could be a nonlinear function, but the activation is the identity function in our implementation:

$${f}_{identity} ( x ) = x .$$

The input tensor dimensions are: (batch_size, n_filters_spat, T′, 1) and the output tensor dimensions: (batch_size, n_fiters_spat, T′, 1)

The structure of subsequent blocks beyond the first block is next discussed. These subsequent blocks, indexed by i, i∈(0, 1, 2, 3) are applied sequentially. Each subsequent block, beyond the first block, consists of a series of layers which perform different operations. The general structure of blocks that follow the first block is: (1) dropout layer; (2) convolutional layer; (3) batch or layer normalization; (4) non-linear activation; (5) pooling layer; and (6) non-linear pooling. These layers are applied sequentially, with the output of one layer serving as the input to the next layer. This process is repeated for each block in the network.

Dropout is applied after each convolutional layer with a specified dropout probability, drop_prob. During training:

$${Y}_{n , c , t , 1} = \{ \begin{matrix}{X}_{n , c , t , 1} / ( 1 - drop\_prob ) , & with probability ( 1 - drop\_prob ) \\ 0 , & with probability drop\_prob\end{matrix}$$

During testing:

$${Y}_{n , c , t , 1} = {X}_{n , c , t , 1} .$$

Dropout does not affect the tensor dimensions. Input tensor dimensions=Output tensor dimensions: (batch_size, n_filters_spat, T′, 1).

The convolution in the i^{th} block is a 1D convolution that operates on the temporal (across the time points, i.e., the third) dimension of the tensor. It is applied over the input tensor, and it learns and detects local features in the input. Unlike the first block, there is no spatial convolution in the later blocks. The input tensor has a shape of (batch_size, Cᵢₙ, Tᵢₙ, 1). The convolutional layer in the i^{th} block uses filters with a shape of (n_filters, 1, filter_lengthᵢ, 1), where n_filtersᵢ and filter_lentgthᵢ depend on the i^{th} block.

$${Y}_{n , k ′ , j , 0} = {( X * {W}_{i} )}_{n , k ′ , j , 0} + {b}_{k ′} = {\sum }_{k = 0}^{filter \_ length \_ i - 1} {X}_{n , k ′ , j + k , 0} \cdot {W}_{i , k ′ , 0 , k , 0} + {b}_{k ′}$$

where: X is the input tensor of shape (N, Cᵢₙ, Tᵢₙ, 1); Wᵢ is the filter of shape (n_filtersᵢ, 1, filter_lengthᵢ, 1); b_{k′} is the bias term for the k′-th filter, and Y is the output tenser of shape (batch_size, n_filtersᵢ, T′ᵢₙ, 1), where T′ᵢₙ=Tᵢₙ−filter_lengthᵢ+1.
Ranges for the indices are:

$$n ∈ [ 0 , batch\_size - 1 ] \\ {k}^{′} ∈ [ 0 , {n\_filters}_{i} - 1 ] \\ j ∈ [ 0 , {T}_{in}^{′} - 1 ]$$

The pooling layer in the i^{th} block is applied after the convolution, batch or layer normalization, and non-linear activation layers. It's there to reduce the spatial dimensionality of the input tensor, which has a shape of (batch_size, n_filtersᵢ, T′ᵢₙ_{ᵢ}, 1). The pooling operation used in the i^{th} block can be either max or mean, depending on the later_pool_mode parameter. Input tensor dimensions are: (batch_size, n_filtersᵢ, T′ᵢₙ_{ᵢ}, 1) and output tensor dimensions: (batch_size, n_filtersᵢ, T′ₒᵤₜ_{ᵢ}, 1), where

$${T}_{{out}_{i}}^{′} = ⌊ \frac{{T}_{{in}_{l}}^{′} - pool\_time \_length}{pool\_time \_stride} + 1 ⌋ .$$

The final convolutional layer acts as a classifier, applying filters to generate class scores. These scores are not (yet) probabilities.

The classifier kernel shape is given by:

(1,n_classes,T″,1),

where T″ is the remaining time dimension (the third dimension of the input tensor X) after all previous layers have been applied. The convolution operation can be expressed as:

$${Y}_{b , c ′ , 0 , 0} = {\sum }_{c = 0}^{C ″ - 1} {\sum }_{t = 0}^{T ″ - 1} {X}_{b , c , t , 1} \cdot {W}_{0 , c ′ , t , 0}$$

where C″ represents the remaining number of feature maps dimension (the second dimension of the input tensor X) after all the previous layers have been applied, and X is the input tensor of shape (batch_size, C″, T″, 1), W is the classifier filter of shape (1, n_classes, T″, 1), and Y is the output tensor of shape (batch_size, n_classes, 1, 1).

The Log Softmax activation function is applied to the output of the convolutional classifier. It normalizes the class scores to probabilities by using the Softmax function, and then computes the logarithm. The Log Softmax function can be expressed as:

$${S}_{b , c ′ , 0 , 0} = log ( \frac{{e}^{{Y}_{b , c ′ , 0 , 0}}}{{\sum }_{j = 0}^{n \_ classes - 1} {e}^{{Y}_{b , j , 0 , 0}}} ) ,$$

where Y is the output tensor of the convolutional classifier of shape (batch_size, n_classes, 1, 1), and S is the output tensor after applying the Log Softmax function, with the same shape as Y.

The squeeze operation is applied to the output of the Log Softmax layer to remove the unnecessary dimensions of size 1. The Squeeze operation can be expressed as:

$${S}_{b , c ′}^{′} = {S}_{b , c ′ , 0 , 0}$$

where S is the output tensor of the Log Softmax layer of shape (batch_size, n_classes, 1, 1), and S′ is the output tensor after applying the Squeeze operation, with shape (batch_size, n_classes).

Finally,

$$Prob [ class = k ] = \frac{{e}^{{S}_{b , k}^{′}}}{{\sum }_{j = 0}^{n \_ classes - 1} {e}^{{S}_{b , j}^{′}}} .$$

By construction, these probabilities are positive and sum to 1. Training the model initial weights for the parameters described in the model architecture. In this subsection, the initialization of weights, the optimization (search to learn the model parameters), and the loss function that is optimized are discussed.

The Xavier initialization, a weight initialization technique that aims to balance the variance of the input and output activations of a layer, is used in one implementation. It is advantageous for deep neural networks, where the standard initialization methods may lead to vanishing or exploding gradients. For a fully connected layer with nᵢₙ input units and nₒᵤₜ output units, the Xavier initialization sets the weights to be randomly chosen from a uniform distribution:

$$W ∼ U ( - \frac{\sqrt{6}}{\sqrt{{n}_{in} + {n}_{out}}} , \frac{\sqrt{6}}{\sqrt{{n}_{in} + {n}_{out}}} )$$

For a convolutional layer with nᵢₙ input channels, nₒᵤₜ output channels, and kernel dimensions kₕ×k_{w}, the Xavier initialization sets the weights to be randomly chosen from a uniform distribution:

$$W ∼ U ( - \frac{\sqrt{6}}{\sqrt{{n}_{in} \cdot {k}_{h} \cdot {k}_{w} + {n}_{out} \cdot {k}_{h} \cdot {k}_{w}}} , \frac{\sqrt{6}}{\sqrt{{n}_{in} \cdot {k}_{h} \cdot {k}_{w} + {n}_{out} \cdot {k}_{h} \cdot {k}_{w}}} )$$

The Adaptive Moment Estimation (“Adam”) method is used. Let θ represent the model parameters (weights and biases). gₜ denote the gradient of the loss function with respect to θ at iteration t, and α be the learning rate. Adam maintains two moving averages for each parameter, mₜ and vₜ, which are initialized to 0. These moving averages are updated using the gradient gₜ and the decay rates β₁ and β₂, as follows:

$$\text{?}$$

$$\text{?}$$

$$\text{?} \text{indicates text missing or illegible when filed}$$

The optimizer uses bias-corrected estimates of the first and second moments:

$$\text{?}$$

$$\text{?}$$

$$\text{?} \text{indicates text missing or illegible when filed}$$

Finally, the model parameters are updated:

$${θ}_{t + 1} = {θ}_{t} - α \frac{{\hat{m}}_{t}}{\sqrt{{\hat{v}}_{t} + ϵ}}$$

The Cross-Entropy Loss function is used in one implementation:

$$L ( θ ) - \frac{1}{batch\_size} \text{?} {{\sum }^{n \_ classes}}_{j = 1} {y}_{ij} log ( {\hat{y}}_{i j} )$$

$$\text{?} \text{indicates text missing or illegible when filed}$$

where yᵢⱼ is the true label for the i^{th} sample and the j^{th} class, and ŷᵢⱼ is the predicted probability for the i^{th} sample and the j^{th} class. The optimization process includes iterating through the dataset for multiple epochs. An epoch is a complete pass through the dataset. The optimization terminates when a predefined number of epochs is reached, which is the default termination procedure.

Backpropagation is the method used to calculate the gradients of the loss function with respect to the model parameters (weights and biases). The gradients are then used by the optimizer (e.g., Adam to update the model parameters, minimizing the loss function. Backpropagation involves performing a forward pass through the network, computing the output of each layer given the input of the previous layer, until the final output is calculated. Compute the loss using the network's output and the true labels. Then, the gradient of the loss function with respect to the output of the final layer is calculated. For each layer in the network, starting from the last and moving towards the input: (1) calculate the gradients of the loss function with respect to the layer's weights and biases using the chain rule; (2) compute the gradient of the loss function with respect to the output of the previous layer (i.e., the input of the current layer). Update the model parameters (weights and biases) using the calculated gradients and the optimizer's update rule.

In a forward pass, the output of each layer given the input of the previous layer is computed until the final output is calculated. The loss is then computed by using the network's output and the true labels, calculate the loss function:

$$L ( θ ) = - \frac{1}{batch\_size} \text{?} {\sum }_{j = 1}^{n \_ classes} {y}_{ij} log ( {\hat{y}}_{i j} )$$

$$\text{?} \text{indicates text missing or illegible when filed}$$

Here, ŷᵢⱼ represents the predicted output (probability) of the j-th class for the i-th sample in the dataset and N represents the number of samples in a batch, i is the index of the sample in the dataset, and j is the index of the class in the multi-class classification problem. The gradient of the loss function is computed with respect to the output of the Final layer:

$$\frac{\partial L}{\partial {\hat{y}}_{i j}}$$

The gradient of L with respect to ŷᵢⱼ is computed as:

$$\frac{\partial L}{\partial {\hat{y}}_{i j}} = - \frac{1}{batch\_size} {\sum }_{i = 1}^{batch \_ size} {\sum }_{j = 1}^{n \_ classes} {y}_{ij} \frac{\partial }{\partial {\hat{y}}_{i j}} log ( {\hat{y}}_{i j} )$$

To compute the derivative, the term inside the summation is considered:

$$\frac{\partial }{\partial {\hat{y}}_{i j}} log ( {\hat{y}}_{i j} ) = \frac{1}{{\hat{y}}_{i j}}$$

The gradient is rewritten as:

$$\frac{\partial L}{\partial {\hat{y}}_{i j}} = - \frac{1}{batch\_size} {\sum }_{i = 1}^{batch \_ size} {\sum }_{j = 1}^{n \_ classes} {y}_{ij} \frac{1}{{\hat{y}}_{i j}}$$

This gradient is computed for each element of the output of the final layer, resulting in a matrix of gradients with the same dimensions as the output. This gradient calculation provides the starting point for computing gradients with respect to the weights and biases of the network layers. In a backward pass, for each layer in the network, starting from the last and moving towards the input:
(1) Calculate the Gradients of the Loss Function with Respect to the Layer's Weights and Biases Using the Chain Rule:

$$\frac{\partial L}{\partial {w}_{i}} = \frac{\partial L}{\partial {y}_{i}} \cdot \frac{\partial {y}_{i}}{\partial {w}_{i}}$$

$$\frac{\partial L}{\partial {b}_{i}} = \frac{\partial L}{\partial {y}_{i}} \cdot \frac{\partial {y}_{i}}{\partial {b}_{i}}$$

where yₗ denotes the output of the l-th layer in the neural network; wₗ denotes the weights of the l-th layer in the neural network; and bₗ denotes the biases of the l-th layer in the neural network.
(2) Compute the Gradient of the Loss Function with Respect to the Output of the Previous Layer (i.e., the Input of the Current Layer):

$$\frac{\partial L}{\partial {y}_{i - 1}} = \frac{\partial L}{\partial {y}_{i}} . \frac{\partial {y}_{i}}{\partial {y}_{i - 1}}$$

To compute these gradients, it is necessary to find the partial derivatives

$$\frac{\partial {y}_{i}}{\partial {w}_{i}} , \frac{\partial {y}_{i}}{\partial {b}_{i}} , and \frac{\partial {y}_{i}}{\partial {y}_{i - 1}} .$$

The specific expressions for these derivatives depend on the type of layer (e.g., convolutional, fully connected, activation function) and the operations performed within the layer. The gradients are then used to update the layer's weights and biases in step 5, following the optimizer's update rule. The model parameters are updated by using the calculated gradients and the optimizer's update rule (e.g., Adam):

$${θ}_{t + 1} = {θ}_{t} - α \frac{{\hat{m}}_{t}}{\sqrt{{\hat{v}}_{t}} + ϵ}$$

During the training process, a loss function, which measures the discrepancy between the predicted output and the true target values, is minimized. The optimization is performed using a method like Stochastic Gradient Descent (SGD) or its variants such as Adam. Training is performed in multiple iterations called epochs. In each epoch, the entire dataset is passed through the network. Recall that to improve computational efficiency and convergence properties of the optimization, the dataset has been divided into smaller subsets called batches, where a batch typically consists of a fixed number of samples, denoted as batch_size. Optimization updates the network weights based on the average gradient of the loss function with respect to the weights, computed over the samples in the batch. The training process iterates over multiple epochs until a stopping criterion is met, such as reaching a predefined number of epochs, or observing no significant improvement in the validation loss.

There are important applications that require regression models or multi-output regression models. The current example involves predicting two regressands: the severity of anxiety level and the severity of mind wandering, given EEG data. The most popular measure of generalized anxiety disorder (GAD) is the Generalized Anxiety Disorder 7-item (GAD-7) scale. The GAD-7 is a self-report questionnaire developed to assess the severity of GAD symptoms in individuals. It is widely used in both clinical and research settings due to its reliability, validity, and ease of administration. The GAD-7 consists of 7 items that ask respondents to rate the frequency of their anxiety symptoms over the past two weeks on a scale from 0 (not at all) to 3 (nearly every day). The total score ranges from 0 to 21, with higher scores indicating greater severity of anxiety symptoms. The Daydreaming Frequency Scale (DDFS) measures the frequency of daydreaming or mind wandering in daily life. Participants rate the frequency of various daydreaming experiences on a scale from 1 (never) to 5 (very often). Suppose that there are EEG recording data for a collection of subjects, as well as labels for each subject for GAD-7 and DDFS, Then one might seek a model that can allow us to predict both of these measures, given a subject's EEG data. The softmax layer is removed. Since the domain is now regression instead of classification, the output of the model into probabilities is not needed. The softmax layer is removed and retain the output of the convolutional classifier. The loss Function is changed by replacing the current loss function (cross-entropy, an appropriate loss for classification) with the Mean Squared Error (MSE) loss. The MSE loss can be computed as:

$$MSE = \frac{1}{N} {\sum }_{i = 1}^{N} {( {y}_{i} - {\hat{y}}_{i} )}^{2}$$

where yᵢ represents the true output. ŷᵢ represents the predicted output, and N is the total number of samples. The output layer is updated to ensure that the output layer has the correct number of neurons (n_dependent_variables) to match the desired number of outputs for regression. After making these modifications, the adapted model can handle n_dependent_variable regression. These ideas are discussed in greater detail below.

The final convolutional layer acts as a regression output layer, applying filters to generate output values. These output values are not probabilities, as the task is now regression. The regression output kernel shape is given by:

$$( 1 , n\_dependent \_variables , {T}^{″} , 1 ) ,$$

where T″ is the remaining time dimension (the third dimension of the input tensor X) after all previous layers have been applied, and n_dependent_variables is the number of output values. The convolution operation can be expressed mathematically as:

$${Y}_{b , c ′ , 0 , 0} = {\sum }_{c = 0}^{C ″ - 1} {\sum }_{c = 0}^{T ″ - 1} {X}_{b , c , t , 1} \cdot {W}_{0 , c ′ , t , 0}$$

where C″ represents the remaining number of feature maps dimension (the second dimension of the input tensor X) after all the previous layers have been applied, and where X is the input tensor of shape (batch_size, C″, T″, 1). W is the regression output filter of shape (1, n_dependent_variables, T″, 1), and Y is the output tensor of shape (batch_size, n_dependent_variables, 1, 1).

The squeeze operation is applied to the output of the convolutional regression output layer to remove the unnecessary dimensions of size 1. The Squeeze operation can be expressed mathematically as:

$${Y}_{b , c ′}^{′} = {Y}_{b , c ′ , 0 , 0}$$

where Y is the output tensor of the convolutional regression output layer of shape (batch_size, n_dependent_variables, 1, 1), and Y′ is the output tensor after applying the Squeeze operation, with shape (batch_size, n_dependent_variabies).

Enhancements to improve the robustness of the solutions, two methods may be used. These include averaging the last n_{last_epochs} epochs. After the loss function has flattened out, indicating that the model has reached a stable state, average the model's output over the last n_{last_epochs} epochs. This will help to reduce the impact of any transient fluctuations in the model's performance that may occur near the end of training, thereby improving the robustness of the solution. The enhancements also include training different models using different random seeds and averaging model output: Train multiple models with different random seeds, ensuring that each model starts with a different initial state. By averaging the output of these models, the risk of overfitting to any specific initial conditions is reduced, and a more robust ensemble model that is less sensitive to the choice of random seed is created. By applying these two methods, the robustness of the model's solutions can be improved, leading to more reliable and consistent performance in the face of varying initial conditions and random fluctuations during training.

The hyperparameters already described above can be extended to include modeling choices including the number of blocks, the type of convolutions, and others. For example, the convolutions were all effectively one-dimensional. 2D convolutions can produce better performance in certain cases. Model performance can be further improved by optimizing the hyperparameters of the model. One effective method for hyperparameter optimization is Sequential Model-based Configuration (“SMC”). SMC is an optimization that uses Bayesian optimization in combination with an acquisition function to guide the search for optimal hyperparameters.

To apply SMC for hyperparameter optimization, the following steps are employed. The hyperparameter search space is defined by specifying the range of possible values for each hyperparameter. This search space should cover the potential combinations of hyperparameters that may yield the best model performance. Models are trained and evaluated by training models with different combinations of hyperparameters sampled from the search space, and evaluate their performance using a predefined performance metric (e.g., mean squared error for regression tasks). The surrogate model is updated by using the results of the trained and evaluated models to update the surrogate model, which estimates the performance of different hyperparameter combinations. In the case of Gaussian Processes (GP), the surrogate model is a GP that models the relationship between the hyperparameters and the performance metric. The GP is defined by a mean function m(x) and a covariance function (kernel) k(x, x′), and it models the performance ƒ(x) as:

$$f ( x ) ∼ 𝒢 𝒫 ( m ( x ) , k ( x , {x}^{′} ) )$$

Given a set of observed hyperparameter combinations X and their corresponding performance metrics y, the posterior distribution of the GP can be computed, which serves as the updated surrogate model. New hyperparameter combinations are selected by using an acquisition function (e.g., Expected Improvement, EI) to select new hyperparameter combinations from the search space, based on the updated surrogate model. The EI acquisition function is defined as:

$$EI ( x ) = E [ max ( f ( x ) - f ( {x}^{+} ) , 0 ) \vert X , y ]$$

where ƒ(x⁺) is the best observed performance so far. The next hyperparameter combination to evaluate is the one that maximizes the EI:

$${x}_{next} = arg {max}_{x} EI ( x )$$

The final three steps are repeated until a stopping criterion is met e.g., a maximum number of iterations or a predefined performance threshold). By applying SMC for hyperparameter optimization, the model performance can be improved as the method iteratively refines the choice of hyperparameters, ultimately yielding a better-performing model configuration.

After training, the model is capable of making predictions on new data. To make a prediction, new input data X_test is fed into the model, which outputs the predicted value using the learned weights and biases. In this section, the form for the idiosyncratic model is motivated and explained in some detail. The side-information model, which is estimated from all data from all prior subjects, is the basis for our estimate of the idiosyncratic model, which incorporates the effects of the hidden information, u, for the particular subject. The idiosyncratic model makes use of a model constructed from the observed discrepancies between the results of the experiments performed ƒ_expensive(v, x, u) and model_{side-info}(v, x).

$$δ ( v ) ≡ f\_expensive ( v , x , u ) - {model}_{side - info} ( v , x ) .$$

The idea is to find a deformation of the side information model that would produce idiosyncratic function values consistent with the observed difference data. δ(v). There are penalties associated with both the extent of the deformation as we i as the degree of consistency with the observed difference data. The idiosyncratic model is then be defined as the side information model plus the model for the discrepancy. After enough data have been accumulated, the shape of model_{side-info}(v, x) stabilizes as a function of (v, x). However, the process will have to accommodate situations where there will be a very limited number of experiments. Recall that the minimum of the idiosyncratic model is sought. So it is important that the extended behavior of the difference function that is estimated becomes flat as a function of v for extreme values of v. That way, the remote behavior of the sum of the side-information model and the difference model will “inherit” behavior similar to that of the side-information model for extreme values. Otherwise, spurious idiosyncratic function minimization results might be obtained. To that end, it is assumed that the difference function is logistic in form.

This section describes one way how a model logistic in form can be fit to the observed difference data. Let y_original denote the vector of observed discrepancy values. To make a model that is logistic in form, it is first assumed that the observed values have been resealed to lie between 0.25 and 0.75. That is done by setting

$$min\_rescale , max\_rescale = 0.25 , 0.75 .$$

$$y min , y max = n p . min ( y\_original ) , n p . max ( y\_original ) .$$

and letting

$$y = min\_rescale + ( max\_rescale - min\_rescale ) * ( y\_original - y min ( / ( y max - y min ) .$$

To allow flexibility, in one instantiation, the logit part is a quadratic function of v.

$$logit ( v ) = {v}^{T} Qv + {a}^{T} v + b ,$$

where Q∈Rⁿ^{ᵥ}^{×n}^{ᵥ}, a∈Rⁿ^{ᵥ} and b∈R¹.
That is, a model for the differences of the form

$${model}_{differencs} ( v ; Q , a , b ) ≡ \frac{1}{1 + {e}^{- ( {v}^{T} Qv - {a}^{T} v + b )}}$$

is sought, where the Q, a, and b are the variables used to fit the model. Recall that the idiosyncratic model is constructed after making the observations (vᵢ, yᵢ) An objective function in the model parameters Q, a, and b is sought that takes into account both the closeness of the values y to the model predictions model_{differences}(v; Q, a, b), and the extent to which model_{sideinfo} needs to be deformed. Now that the y values have been rescaled, each pair (y, 1−y) can be viewed as a probability measure on two states, likewise model_{differences}(v; Q, a, b). So, to find parameters Q, a, b that make the model_{differences}(v; Q, a, b) values close to the y values, one can consider the Kullback-Leibler relative entropy, summed over the y values. To find the extent to which model_{sideinfo} is deformed, the norms of Q, a, and b are considered, along with the range of the original observed discrepancy values before rescaling, resulting in

$$λ ( y max - y min ) ( {\sum }_{j = 1}^{{n}_{v}} {\sum }_{k = 1}^{{n}_{v}} {\sum }_{m = 1}^{{n}_{v}} {Q}_{jm} {Q}_{k m} + {\sum }_{l = 1}^{{n}_{v}} {a}_{l}^{2} + {b}^{2} ) ,$$

where λ is a parameter that allows us to weight the deformation terms relative to the closeness terms. The idiosyncratic model is the sum of model_{sideinfo} and model_{differences} transformed back to the original space.

The idiosyncratic model is merely an estimate for the unknown idiosyncratic function and the minimization server as a way to find, by experiment, new points to evaluate to learn more about the idiosyncratic function behavior. It is sometimes useful to do controlled exploration beyond the strict minimum of the function, i.e., to pick a point that is near the strict minimum, but offers new perspective, and yet is somehow far from previous experiment points for this subject. This is accomplished by means of the hermit point method, that provides a point within a small box around the strict minimum point, but as far away as possible from the closed of the prior experiment points. This is accomplished by generating a large number of points in a mesh grid in a small box around the strict minimum, and then extracting a more manageable number of points, B by K means, and then finding the point with greatest minimum distance from the set of prior experiment points, A

$${x}^{*} = arg {max}_{x ∈ B} {min}_{y ∈ A} D ( x , y ) ,$$

where D(x, y) denotes the euclidean distance between x and y.

The present invention has been described in terms of particular embodiments, it is not intended that the invention be limited to these embodiments. Modifications within the spirit of the invention will be apparent to those skilled in the art. For example, any of many different implementations of the DMS and CMS applications can be obtained by varying various design and implementation parameters, including modular organization, control structures, data structures, hardware, operating system, and virtualization layers, and other such design and implementation parameters. The currently disclosed MM/SD system can be implemented in many different ways. The CMS application may receive and process many different types of signals, including physiological signals from physiology sensors, package the signal data into network messages, and forward the network messages to the DMS application. The DMS application can determine attribute values for a wide range of different mental-state attributes. Attribute values ma, indicate the presence or absence of attributes, the presence or absence of attributes that together represent a mental-state parameter with multiple different levels of significance or severity, or attributes that express a severity value as a real number. Many different types of machine-learning and artificial-intelligence technologies can be used for determining attribute values as well as for determining whether or not a stimulus should be provided to a user based on the user's current mental state.

1. A monitoring and stimulus-provision system comprising:
a processor-controlled user system that provides information to a user, includes a client-side application that receives physiological data from one or more sensors and monitors included in, or connected to, the processor-controlled user system, and that provides stimuli to the user in response to received stimulus directives; and
a distributed application that receives physiological data from the client-side application, periodically processes the received physiological data to generate representations of the user's mental state, generates directives for stimulus provision based on the generated representations of the user's mental state, and sends the stimulus directives to the processor-controlled user system.

2. The monitoring and stimulus-provision system of claim 1 wherein the processor-controlled user system provides information to the user in the context of an instructional, therapeutic, or diagnostic session.

3. The monitoring and stimulus-provision system of claim provides information to the user by one or more of:
displaying images on a display-screen component of the user's processor-controlled system;
displaying one or more videos on the display-screen component of the user's processor-controlled system;
displaying text on the display-screen component of the user's processor-controlled system; and
outputting audio signals to an audio-presentation device, including one or more of speakers, a headset, and earbuds.

4. The monitoring and stimulus-provision system of claim 1 wherein the physiological data received by the client-side application includes one or more of:
EEG signals;
video and or still-image data generated by the user's processor-controlled-system camera;
audio data generated by a microphone incorporated in, or connected to, the user's processor-controlled-system;
heartbeat-rate data generated by a heartbeat-rate sensor connected to the user's processor-controlled-system;
temperature data generated by a temperature sensor connected to the user's processor-controlled-system;
blood-oxygen-level data generated by a blood-oxygen-level sensor connected to the user's processor-controlled-system; and
blood-pressure data generated by a blood pressure sensor connected to the user's processor-controlled-system.

5. The monitoring and stimulus-provision system of claim 1 wherein stimuli provided to the user in response to received stimulus directives include one or more of:
peak alpha-wave frequency light signals displayed to a display screen of the user's processor-controlled system;
visual patterns displayed to the display screen of the user's processor-controlled system;
oscillating visual patterns displayed to the display screen of the user's processor-controlled system;
videos or still images displayed to the display screen of the user's processor-controlled system; and
audio played by one or more speakers connected to the user's processor-controlled system.

6. The monitoring and stimulus-provision system of claim 1 wherein the stimuli are provided to the user by the client-side application directing an information-provision application running in the user's processor-controlled system to incorporate the stimuli into instructional, therapeutic, or diagnostic information provided to the user.

7. The monitoring and stimulus-provision system of claim wherein the stimuli are provided to the user by the client-side application issuing commands to one or more operating-system interfaces within the user's processor-controlled system.

8. The monitoring and stimulus-provision system of claim wherein the stimuli are provided to the user by
intercepting, by the distributed application, instructional, therapeutic, or diagnostic information directed from a remote information source to the user's processor-controlled system;
incorporating, by the distributed application, stimulus data into the intercepted instructional, therapeutic, or diagnostic information; and
forwarding, by the distributed application, the intercepted instructional, therapeutic, or diagnostic information with incorporated stimulus data to the user's processor-controlled system.

9. The monitoring and stimulus-provision system of claim 1 wherein the distributed application runs within a distributed computer system that is remote from, and connected by electronic communications to, the user's processor-controlled system.

10. The monitoring and stimulus-provision system of claim 1 wherein the distributed application comprises:
a data store;
a controller that receives and responds to user commands;
one or more signal-processor/stimulus-directors that receive physiological data transmitted to the distributed application by one or more user processor-controlled systems and that generate stimulus directives that are transmitted to more user processor-controlled systems;
a front end that receives network messages from one or more user processor-controlled devices, Forwards received messages containing user commands to the controller, forwards each received message containing physiological data to one of the one or more signal-processor/stimulus-directors, received responses to user commands from the controller and forwards the responses to user commands to user processor-controlled device, and receives stimulus directive from the one or more signal-processor/stimulus-directors and forwards each stimulus directive to a user processor-controlled system.

11. The monitoring and stimulus-provision system of claim 10 wherein each signal-processor/stimulus-director includes:
a demultiplexer that extracts physiological data for each of one or more physiological-data channels from a received physiological-data message received from the front end and outputs the physiological-data for each channel to a channel-specific physiological-data bus;
one or more attribute detectors that each receive physiological data from one or more of the channel-specific physiological-data buses and outputs attribute indications; and
a mental-state synthesizer and stimulus director that receives attribute indications from the one or more attribute detectors, uses the received attribute indications to generate mental-state representations, uses the mental-state representations to determine whether or not a user should receive one or more stimuli, and, when the mental-state synthesizer and stimulus director determines that a user should receive one or more stimuli, generates one or more stimulus directives for the user and forwards the one or more stimulus directives to the front end for transmission to the user's processor-controlled system.

12. The monitoring and stimulus-provision system of claim of claim 11 wherein, for each physiological-data channel, the demultiplexer filters, normalizes, and removes artifacts from the physiological data and partitions the physiological data into data blocks for output to the channel-specific physiological-data buses.

13. The monitoring and stimulus-provision stem of claim 11 wherein each attribute detector
receives physiological-data blocks from one or more channel-specific physiological-data buses and stores the received physiological-data blocks in one or more buffers; and
periodically
removes physiological-data blocks from the one or more buffers;
generates an input vector from the removed physiological-data blocks;
inputs the input vector to a machine-learning analyzer which outputs a corresponding attribute indication; and
outputs the corresponding attribute indication to the mental-state synthesizer and stimulus director.

14. The monitoring and stimulus-provision system of claim 11 wherein a mental-state synthesizer and stimulus director
receives attribute indications from one or more attribute detectors;
buffers the received attribute indications in one or more buffers;
periodically removes attribute indications from the one or more buffers;
generates a meant-state representation using the removed attribute indications;
inputs the mental-state representation to a machine-learning analyzer to determine whether a user should receive one or more stimuli, generates one or more stimulus directives for the user and forwards the one or more stimulus directives to the front end for transmission to the user's processor-controlled system.

15. The monitoring and stimulus-provision system of claim 11 wherein the machine-learning analyzer incorporated into an attribute detector and a mental-state synthesizer and stimulus director is one of:
a neural network;
a neural network and post-processing elements, include a softmax element;
a rule-based system; and
a decision tree.

16. The monitoring and stimulus-provision system of claim 11 wherein an attribute indication includes one or more numerical values related to a mental-state attribute, the one or more numerical values including:
an attribute strength or level;
an attribute-occurrence probability; and
a confidence value.

17. The monitoring and stimulus-provision system of claim 11 wherein a mental-state representation includes values for each of one or more mental-state attributes.

18. A method that monitors and stimulates a user of processor-controlled device while the user is provided information during an instructional, therapeutic, or diagnostic session, the method comprising:
providing a client-side application that runs within the user's processor-controlled device;
providing a distributed application that runs in a distributed computer system;
receiving, by the client-side application, physiological data from one or more sensors and monitors included in, or connected to, the processor-controlled user system;
forwarding, by the client-side application, physiological data to the distributed application;
receiving, by the distributed application, physiological data forwarded from the client-side application;
processing, by the distributed application, the received physiological data to generate representations of the user's mental state;
generating, by the distributed application, directives for stimulus provision based on the generated representations of the user's mental state;
transmission, by the distributed application, the stimulus directives to the processor-controlled user system; and
generation stimuli by the processor-controlled user system.

19. The method of claim 18 wherein the physiological data received by the client-side application includes one or more of:
BEG signals;
video and or still-image data generated by the user's processor-controlled-system camera;
audio data generated by a microphone incorporated in, or connected to, the user's processor-controlled-system;
heartbeat-rate data generated by a heartbeat-rate sensor connected to the user's processor-controlled-system;
temperature data generated by a temperature sensor connected to the user's processor-controlled-system;
blood-oxygen-level data generated by a blood-oxygen-level sensor connected to the user's processor-controlled-system; and
blood-pressure data generated by a blood pressure sensor connected to the user's processor-controlled-system.

20. The method of claim 18 wherein stimuli provided to the user in response to received stimulus directives include one or more of:
peak alpha-wave frequency light signals displayed to a display screen of the user's processor-controlled system;
visual patterns displayed to the display screen of the user's processor-controlled system;
oscillating visual patterns displayed to the display screen of the user's processor-controlled system;
videos or still images displayed to the display screen of the user's processor-controlled system; and
audio played by one or more speakers connected to the user's processor-controlled system.
